(19) United States
(12) Patent Application Publication
(10) Pub. No.: US 2023/0197269 A1
(43) Pub. Date: Jun. 22, 2023

(54) SYSTEMS AND METHODS FOR DETECTING VIRAL DNA FROM SEQUENCING

(71) Applicant: Tempus Labs, Inc., Chicago, IL (US)

(72) Inventors: Robert Tell, Chicago, IL (US); Jerod Parsons, Chicago, IL (US); Stephen J. Bush, Skokie, IL (US); Aly A. Khan, Chicago, IL (US); Ariane Lozac'hmeur, Chicago, IL (US); Denise Lau, Santa Monica, CA (US)

(21) Appl. No.: 17/800,492

(22) PCT Filed: Feb. 18, 2021
(86) PCT No.: PCT/US2021/018619

§ 371 Date: Aug. 17, 2022

Related U.S. Application Data
Application No. PCT/US2021/018619 is a continuation of application No. 16/802,126, filed on Feb. 26, 2020, now Pat. No. 11,043,304.
(60) Provisional application No. 62/978,067, filed on Feb. 18, 2020.

(51) Int. Cl. G16H 50/20; A61K 31/513; A61K 31/337; A61K 33/243; G16H 10/40; G16H 50/70; C07K 16/22; A61K 45/06

(52) U.S. Cl. CPC G16H 50/20; A61K 31/337; A61K 31/513; A61K 33/243; C07K 16/22; G16H 10/40; G16H 50/70; A61K 45/06

(57) ABSTRACT

Methods, systems, and software are provided for determining whether a subject is afflicted with an oncogenic pathogen. Nucleic acids from a biological sample of the subject are hybridized to a probe set that includes probes for human genomic loci and for genomic loci of oncogenic pathogens. Sequence reads of the hybridized nucleic acid are obtained and it’s determined whether each sequence read aligns to a human reference genome. For each sequence read that fails to align to the human reference genome, it’s determined whether the sequence read aligns to a reference genome of an oncogenic pathogen. Sequence reads that both (i) fail to align to the human reference genome and (ii) align to a reference genome of an oncogenic pathogen are tracked, thereby obtaining a sequence read count for the oncogenic pathogen. The sequence read count is used to ascertain whether the subject is afflicted with the oncogenic pathogen.

### CROSS REFERENCE TO RELATED APPLICATIONS

This application claims priority to U.S. Pat. Application No. 16/802,126, filed on Feb. 26, 2020, and U.S. Provisional Pat. Application No. 62/978,067, filed on Feb. 18, 2020, the contents of which are hereby incorporated by reference in their entireties for all purposes.

### TECHNICAL FIELD

The present disclosure relates generally to systems and methods for detecting oncogenic pathogenic infections in cancer patients.

### BACKGROUND

The presence of oncogenic pathogen infections account for 10 to 12% of all cancers. For example, consider the case of gastric cancer, which is the third most common cause of cancer death worldwide, with more than 700,000 deaths estimated to have occurred in 2012. See, Ferlay, et al., 2013, “Cancer Incidence and Mortality Worldwide,” IARC CancerBase 11, [Internet]. Lyon, France: International Agency for Research on Cancer. Beyond genetic factors, gastric carcinogenesis is thought to be associated with multiple environmental factors. Among the environmental factors, increasing evidence suggests that a subset of gastric cancers is associated to Epstein-Barr virus (EBV) infection. See, Burke et al., 1990, “Lymphoepithelial carcinoma of the stomach with Epstein-Barr virus demonstrated by polymerase chain reaction,” Mod Pathol. 3:377–380. In fact, recent cancer genome atlas research has provided a molecular classification defining EBV-positive gastric cancer as a specific subtype. See, 2014, “Cancer Genome Atlas Research Network. Comprehensive molecular characterization of gastric adenocarcinoma,” Nature. 513, pp. 202-09.

As such, the presence of such oncogenic pathogens affects the prognosis of the associated cancer. Accordingly, when a subject has a type of cancer that is known to frequently arise in conjunction with an oncogenic pathogen, knowledge of the pathogen status of the subject is important to have because it may change the treatment options of the subject. For example, numerous clinical trials investigating the benefit of radiation or chemotherapy dose reduction for HPV positive head and neck cancers have shown promising results. Additionally, pathogen-associated tumors are more likely to present higher levels of inflammation and immune infiltration, which make them good candidates for immunotherapy.

A drawback with conventional diagnosis is that, in order to determine whether a subject is afflicted with a particular pathogen, a completely independent assay is performed separate and apart from the assays that were used to diagnose a subject with cancer in the first instance, or used to evaluate a stage of the cancer. For example, in the case of EBV, separate laboratory methods such as in situ hybridization (ISH) or polymerase chain reaction (PCR) for resected tissue, biopsy, or blood, or enzyme-linked immunosorbent assay (ELISA) or immunofluorescence assay (IFA) for serum samples is performed to detect the EBV infection. This is unsatisfactory because it increases the expense of diagnosis and, in some instances, where the pathogen test is only run after a type of cancer that is known to be associated with oncogenic pathogen has been diagnosed, delays the development of a treatment plan for the subject until the pathogen assay results have been obtained.

Given the above background, what is needed in the art are improved systems and methods for pathogen detection that directly determine the presence of a given pathogen detection without a requirement for a separate independent assay for the pathogen detection.

### SUMMARY

Accordingly, improved methods for distinguishing cancers associated with oncogenic pathogen infections that contribute to the cancer pathology and cancers that are not associated with oncogenic pathogen infections are provided. Improved methods are also provided for treating cancer patients based on whether their cancer is associated with an oncogenic pathogen infection. The present disclosure addresses these needs, for example, by providing methods for determining whether a subject is afflicted with an oncogenic pathogen based on sequencing data generated from a biological sample of the subject. In some embodiments, these methods include computational subtraction of human sequence reads prior to alignment of the remaining sequence reads against oncogenic pathogen reference constructs.

One aspect of the present disclosure provides a method of determining whether a subject is afflicted with an oncogenic pathogen. The method includes obtaining sequencing data from a nucleic acid sample isolated from a biological sample of the subject and determining whether each sequence read aligns to a human reference genome. The method then includes determining whether sequence reads that don’t align to the reference human genome align to a reference genome of an oncogenic pathogen. The method also includes, for each respective oncogenic pathogen in a plurality of oncogenic pathogens, tracking the number of sequence reads that (i) fail to align to the human reference genome and (ii) align to the reference genome of the respective oncogenic pathogen, thereby obtaining a sequence read count for each oncogenic pathogen. The method then includes using the sequence read count for each oncogenic pathogen to ascertain whether the subject is afflicted with an oncogenic pathogen.

In some embodiments, the method includes isolating nucleic acids from the biological sample of the subject, and hybridizing the isolated nucleic acids to a probe set including (i) a plurality of nucleic acid probes for a plurality of human genomic loci and (ii) a respective set of nucleic acid probes for genomic loci of each respective oncogenic pathogen in a plurality of oncogenic pathogens.

In some embodiments, determining whether each sequence read aligns to the human reference genome is performed using an index-based alignment algorithm.

In some embodiments the determining, for each respective sequence that does not align to the human reference genome, whether the respective sequence aligns to a reference genome for an oncogenic pathogen is performed by using an index-based alignment algorithm. In some such embodiments, this is further confirmed by performing a competitive alignment against the reference human genome.

In some embodiments, the results of the method are further used to generate a clinical report about the cancer status of the subject. In some embodiments, the clinical report includes information selected from whether the subject is afflicted with cancer, a type of cancer the subject is afflicted with, a primary origin of a cancer the subject is afflicted with, a recommendation for treatment of a cancer the subject is afflicted with, and a prognosis for the subj ect.

In some embodiments, a method is provided for determining whether a subject is afflicted with an oncogenic pathogen by sequencing both DNA and RNA obtained from one or more biological samples from the subject. In some embodiments, the method includes making a first determination of whether the subject is afflicted with an oncogenic pathogen based on the DNA sequencing data, using one or more of the methods disclosed herein, and a second determination of whether the subject is afflicted with an oncogenic pathogen based on the RNA sequencing data, using one or more of the methods disclosed herein, and then combining the first and second determinations to make a final determination of whether the subject is afflicted with an oncogenic pathogen. In some embodiments, the combining includes determining whether both the first determination and the second determination indicate that the subject is afflicted with the oncogenic pathogen and accepting the determination if both indicate that the subject is afflicted with the oncogenic pathogen or rejecting the determination if at least one of the determinations does not indicate that the subject is afflicted with the oncogenic pathogen. In some embodiments, the combining includes determining whether either of the first determination and the second determination indicate that the subject is afflicted with the oncogenic pathogen and accepting the determination if at least one of the determinations indicates that the subject is afflicted with the oncogenic pathogen or rejecting the determination if both of the determinations do not indicate that the subject is afflicted with the oncogenic pathogen. In some embodiments, the first determination and the second determination are each a probability or likelihood that the subject is afflicted with the oncogenic pathogen and the combining includes averaging the probabilities or likelihoods to generate a final probability or likelihood that the subject is afflicted with the oncogenic pathogen.

In some embodiments, a first determination of whether the subject is afflicted with one or more oncogenic pathogens in a first plurality of oncogenic pathogens is made based on DNA sequencing of a biological sample from the subject, according to any of the methods described herein, and a second determination of whether the subject is afflicted with one or more oncogenic pathogens in a second plurality of oncogenic pathogens is made based on RNA sequencing of a biological sample from the subject (e.g., the same biological sample or a different biological sample from the subject), according to any of the methods described herein. In some embodiments, the first plurality of oncogenic pathogens and the second plurality of oncogenic pathogens are the same set of oncogenic pathogens. In some embodiments, the first plurality of oncogenic pathogens and the second plurality of oncogenic pathogens are different sets of oncogenic pathogens. In some embodiments, when the first and second pluralities of oncogenic pathogens are different sets of oncogenic pathogens, there is an overlap between the two sets of oncogenic pathogens. In some embodiments, when the first and second pluralities of oncogenic pathogens are different sets of oncogenic pathogens and there is an overlap in the two sets of oncogenic pathogens, a single determination that the subject is afflicted with an oncogenic pathogen that is part of both sets is sufficient to call the pathogenic infection. In other embodiments, when the first and second pluralities of oncogenic pathogens are different sets of oncogenic pathogens and there is an overlap in the two sets of oncogenic pathogens, a single determination that the subject is afflicted with an oncogenic pathogen that is part of both sets is not sufficient to call the pathogenic infection, but a single determination that the subject is afflicted with a second oncogenic pathogen that is part of only one of the two sets is sufficient to call the second pathogenic infection. In some embodiments, when the first and second pluralities of oncogenic pathogens are different sets of oncogenic pathogens, there is no overlap in the two sets of oncogenic pathogens.

Other embodiments are directed to systems, portable consumer devices, and computer readable media associated with the methods described herein.

As disclosed herein, any embodiment disclosed herein when applicable can be applied to any aspect.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIGS. 1A and 1B collectively illustrates a block diagram of an example of a computing device for determining whether a subject is afflicted with an oncogenic pathogen, in accordance with some embodiments of the present disclosure.

FIG. 2 illustrates an example of a distributed diagnostic environment for determining whether a subject is afflicted with an oncogenic pathogen, in accordance with some embodiments of the present disclosure.

FIG. 3 provides a flow chart of processes and features for determining whether a subject is afflicted with an oncogenic pathogen, in which optional blocks are indicated with dashed boxes, in accordance with some embodiments of the present disclosure.

FIGS. 4A and 4B collectively provide a list of example genes that are informative for classifying cancer in a subject, in accordance with some embodiments of the present disclosure.

FIGS. 5A, 5B, 5C, 5D, 5E, 5F, 5G, 5H, 51, and 5J collectively provide a flow chart of processes and features for determining whether a subject is afflicted with an oncogenic pathogen, in which optional blocks are indicated with dashed boxes, in accordance with some embodiments of the present disclosure.

FIGS. 6A and 6B collectively illustrate a block diagram of an example computing device, in accordance with some embodiments of the present disclosure.

FIGS. 7A, 7B, 7C, 7D, and 7E collectively provide a flow chart of processes and features for training a classifier to discriminate between a first cancer condition associated with infection by a first oncogenic pathogen and a second cancer condition associated with an oncogenic pathogen-free status, in which optional blocks are indicated with dashed boxes, in accordance with some embodiments of the present disclosure.

FIG. 8 provides a flow chart of processes and features for discriminate between a first cancer condition associated with infection by a first oncogenic pathogen and a second cancer condition associated with an oncogenic pathogen-free status, and optionally treating the cancer condition based on the oncogenic pathogen status of the cancer, in accordance with some embodiments of the present disclosure.

FIG. 9A provides a breakdown of the compositions of the TCGA training and the testing datasets for training a classifier to discriminate between a first cancer condition associated with an HPV oncogenic viral infection and a second cancer condition not associated with an HPV oncogenic viral infection, in accordance with some embodiments of the present disclosure.

FIG. 9B illustrates features of a cancerous tissue that are useful for discriminating between a first cancer condition associated with an HPV oncogenic viral infection and a second cancer condition not associated with an HPV oncogenic viral infection, in accordance with some embodiments of the present disclosure.

FIG. 9C illustrates performance metrics for a trained support vector machine, against the training dataset, for discriminating between a first cancer condition associated with an HPV oncogenic viral infection and a second cancer condition not associated with an HPV oncogenic viral infection, in accordance with some embodiments of the present disclosure.

FIG. 9D illustrates performance metrics for a trained support vector machine, against a validation dataset, for discriminating between a first cancer condition associated with an HPV oncogenic viral infection and a second cancer condition not associated with an HPV oncogenic viral infection, in accordance with some embodiments of the present disclosure.

FIG. 10A provides a breakdown of the compositions of the TCGA training and the testing datasets for training a classifier to discriminate between a first cancer condition associated with an EBV oncogenic viral infection and a second cancer condition not associated with an EBV oncogenic viral infection, in accordance with some embodiments of the present disclosure.

FIG. 10B illustrates features of a cancerous tissue that are useful for discriminating between a first cancer condition associated with an EBV oncogenic viral infection and a second cancer condition not associated with an EBV oncogenic viral infection, in accordance with some embodiments of the present disclosure.

FIG. 10C illustrates performance metrics for a trained support vector machine, against the training dataset, for discriminating between a first cancer condition associated with an EBV oncogenic viral infection and a second cancer condition not associated with an EBV oncogenic viral infection, in accordance with some embodiments of the present disclosure.

FIG. 10D illustrates performance metrics for a trained support vector machine, against a validation dataset, for discriminating between a first cancer condition associated with an EBV oncogenic viral infection and a second cancer condition not associated with an EBV oncogenic viral infection, in accordance with some embodiments of the present disclosure.

FIG. 11A illustrates principal component analysis of expression features of the genes identified in Example 3 to be differentially expressed in head and neck and cervical cancers associated with an HPV viral infection, in tissue samples of head and neck and cervical cancers, in accordance with some embodiments of the present disclosure.

FIG. 11B illustrates principal component analysis of expression features of genes identified in Example 4 to be differentially expressed in gastric cancers associated with an EBV viral infection, in tissue samples of head and neck and cervical cancers, in accordance with some embodiments of the present disclosure.

FIG. 12A illustrates an example report for an HPV positive head and neck squamous cancer, in accordance with some embodiments of the present disclosure.

FIG. 12B illustrates an example report for an HPV positive cervical cancer, in accordance with some embodiments of the present disclosure.

Like reference numerals refer to corresponding parts throughout the several views of the drawings.

### DETAILED DESCRIPTION

The present disclosure provides systems and methods useful for determining whether a subject is afflicted with an oncogenic pathogen. The present disclosure further provides systems and methods useful for treating cancer patients, based on whether their cancer is associated with an oncogenic pathogen infection or not.

For example, in one aspect, the present disclosure provides systems and methods for determining whether a subject is afflicted with an oncogenic pathogen based on data generated for the classification of a cancer in a subject. As described herein, in some embodiments, the method includes using sequencing data that is generated by probe-based capture of nucleic acids from a biological sample from the subject. Advantageously, employing a single assay for cancer classification and oncogenic pathogen detection decreases the time, capital, and resources needed to provide comprehensive information about the cancer status of a patient. This is in contrast with conventional methods for detecting oncogenic pathogens that require a separate assay solely dedicated to the oncogenic pathogen detection, and which require additional resources beyond those used to classify a subject’s cancer status and/or take additional time to obtain thereby delaying development of a treatment plan.

In some embodiments, the sequence reads are first aligned against a reference human genome and then sequences that do not align to the human genome are aligned against reference sequences, e.g., all or portions of reference pathogenic genomes, of one or more oncogenic pathogens. Advantageously, pre-filtering the sequence reads by removing those that align to the reference human genome greatly decreases the time needed to perform the auxiliary alignments against the pathogenic genomes, particularly when many pathogenic genomes are being sampled.

### Definitions

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the term “subject” refers to any living or non-living human. In some embodiments, a subject is a male or female of any stage (e.g., a man, a women or a child).

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map sequence reads obtained from sequencing a sample from the subject. A reference genome can refer to a haploid or diploid genome to which sequence reads from the biological sample and a constitutional sample can be aligned and compared. An example of constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “locus” refers to a position (e.g., a site) within a genome, e.g., on a particular chromosome. In some embodiments, a locus refers to a single nucleotide position within a genome, i.e., on a particular chromosome. In some embodiments, a locus refers to a small group of nucleotide positions within a genome, e.g., as defined by a mutation (e.g., substitution, insertion, or deletion) of consecutive nucleotides within a cancer genome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “allele” refers to a particular sequence of one or more nucleotides at a chromosomal locus.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “mutation,” refers to a detectable change in the genetic material of one or more cells. In a particular example, one or more mutations can be found in, and can identify, cancer cells (e.g., driver and passenger mutations). A mutation can be transmitted from apparent cell to a daughter cell. A person having skill in the art will appreciate that a genetic mutation (e.g., a driver mutation) in a parent cell can induce additional, different mutations (e.g., passenger mutations) in a daughter cell. A mutation generally occurs in a nucleic acid. In a particular example, a mutation can be a detectable change in one or more deoxyribonucleic acids or fragments thereof. A mutation generally refers to nucleotides that is added, deleted, substituted for, inverted, or transposed to a new position in a nucleic acid. A mutation can be a spontaneous mutation or an experimentally induced mutation. A mutation in the sequence of a particular tissue is an example of a “tissue-specific allele.” For example, a tumor can have a mutation that results in an allele at a locus that does not occur in normal cells. Another example of a “tissue-specific allele” is a fetal-specific allele that occurs in the fetal tissue, but not the maternal tissue.

As used herein the term “cancer,” “cancerous tissue,” or “tumor” refers to an abnormal mass of tissue in which the growth of the mass surpasses and is not coordinated with the growth of normal tissue. A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

As used herein, a “cancer condition associated with an oncogenic pathogen infection,” either generically or with reference to a specific oncogenic pathogen, refers to the condition in which a cancer subject, afflicted with a specific cancer, is further afflicted with a pathogen (e.g., virus) known to associate with the specific cancer.

As used herein, a “cancer condition that is not associated with an on oncogenic pathogen infection,” either generically or with reference to a specific oncogenic pathogen, refers to the condition in which a cancer subject, afflicted with a specific cancer, is specifically not afflicted with a pathogen (e.g., virus) known to associate with the specific cancer.

As used herein, the terms “sequencing,” “sequence determination,” and the like as used herein refers generally to any and all biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read segment” or “read” refers to any nucleotide sequences including sequence reads obtained from an individual and/or nucleotide sequences derived from the initial sequence read from a sample obtained from an individual. For example, a read segment can refer to an aligned sequence read, a collapsed sequence read, or a stitched read. Furthermore, a read segment can refer to an individual nucleotide base, such as a single nucleotide variant.

As used herein, the term, “reference exome” refers to any particular known, sequenced or characterized exome, whether partial or complete, of any tissue from any organism or pathogen that may be used to reference identified sequences from a subject. Example reference exomes used for human subjects as well as many other organisms are provided in the on-line genome browser hosted by the National Center for Biotechnology Information (“NCBI”).

As used herein, the term “reference genome” refers to any particular known, sequenced or characterized genome, whether partial or complete, of any organism or pathogen that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species’ set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “minimum edit distance” refers to the minimum number of editing operations required to change one sequence, e.g., a locus within a reference genome, to exactly match another sequence, e.g., a sequence read. With reference to the editing of a locus of a reference genome to match a sequence read, possible editing operations include inserting a nucleotide (e.g., where an alignment between the sequences shows that a gap must exist in the reference sequence in order to align with the sequence read), deleting a nucleotide (e.g., where an alignment between the sequences shows that a gap must exist in the sequence read in order to align to the reference sequence), and substituting one nucleotide for another (e.g., where an alignment between the sequences shows that there is a mismatch at a particular nucleic acid position). In some embodiments, weights are independently assigned to each editing operation when calculating a minimal editing distance score between two sequences, in order to prioritize the importance of one or more particular types of editing operations relative to the other editing operations.

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

The term “classification” can refer to any number(s) or other characters(s) that are associated with a particular property of a sample. For example, a “+” symbol (or the word “positive”) can signify that a sample is classified as having deletions or amplifications. In another example, the term “classification” can refer to an oncogenic pathogen infection status, an amount of tumor tissue in the subject and/or sample, a size of the tumor in the subject and/or sample, a stage of the tumor in the subject, a tumor load in the subject and/or sample, and presence of tumor metastasis in the subject. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Example System Embodiments

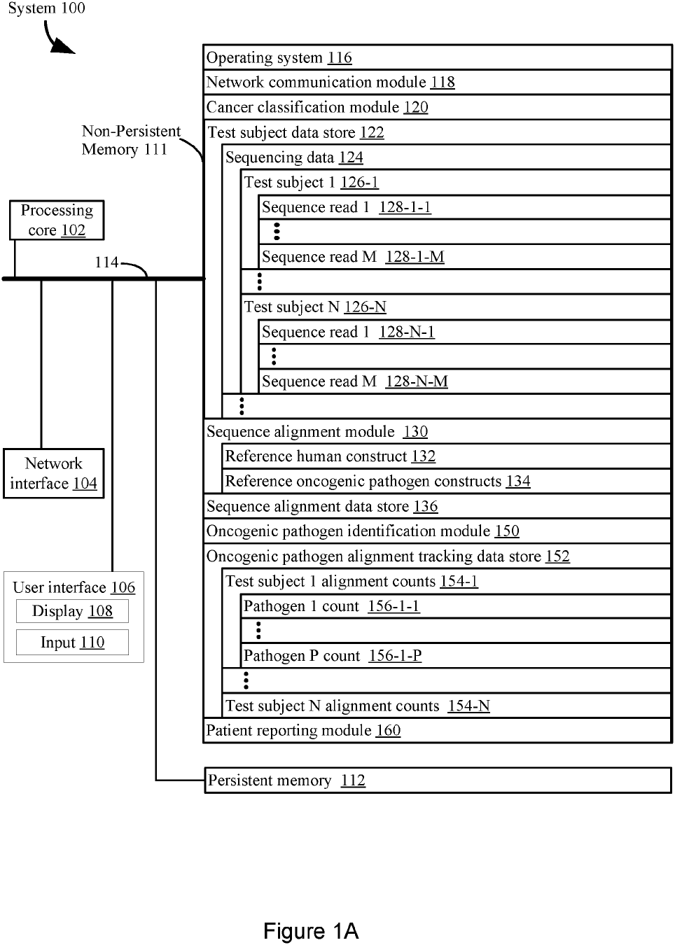

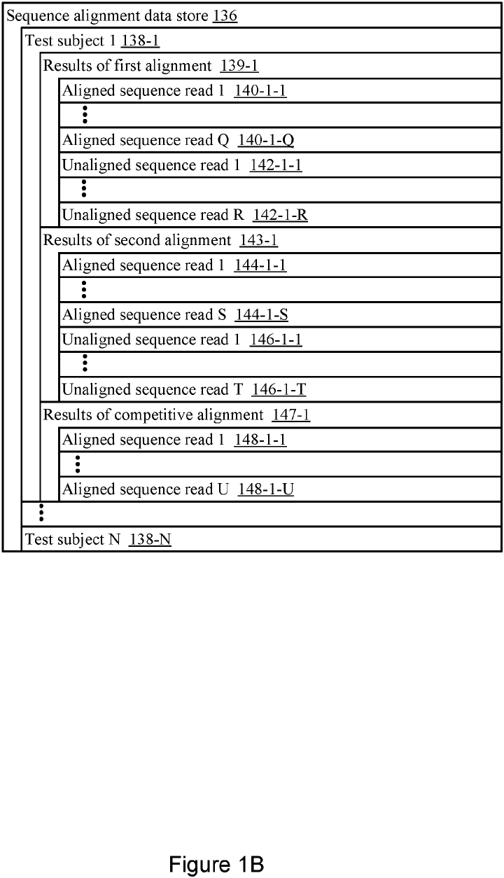

DNA sequencing-based pathogen detection - Now that an overview of some aspects of the present disclosure and some definitions used in the present disclosure have been provided, details of an exemplary system are now described in conjunction with FIG. 1. FIG. 1 is a block diagram illustrating a system 100 in accordance with some implementations. The device 100 in some implementations includes one or more processing units CPU(s) 102 (also referred to as processors), one or more network interfaces 104, a user interface 106, a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an optional operating system 116, which includes procedures for handling various basic system services and for performing hardware dependent tasks;
an optional network communication module (or instructions) 118 for connecting the system 100 with other devices and/or a communication network 212;
a cancer classification module 120 for classifying the cancer status of a subject based on test subject data, e.g., sequencing data 124 stored in test subject data store 122;
a test subject data store 122 for storing datasets containing biological information about test subjects, including sequencing data 124, e.g., sequence reads 128 from one or more test subjects 126 (in some embodiments, one or more data sets stored in subject data store 122 include information about one or more of the pathology of a tissue sample from the subject, genomic information about the subject, exomic information about the subject, epigenetic information about the subject, phenomic information about the subject, proteomic information about the subject, metabolomics information about the subject, and personal characteristics of the subject);
a sequence alignment module 130 for aligning sequencing data 124 to a reference human construct (e.g., genome or exome) 132 and reference pathogen constructs (e.g., whole or partial genomes or exomes) 134 (in some embodiments, the reference human construct and/or reference oncogenic pathogen constructs are stored on a remote server and accessed by system 100);
a sequence alignment data store 136 for storing the results of first alignment 139 between sequence reads 128 of a test subject 138 and reference human construct 132 (e.g., alignments 140 and unaligned sequence reads 142), second alignment 143 between sequence reads 142 that did not align to the human reference construct and oncogenic pathogen reference constructs 134 (e.g., alignments 144 and unaligned sequence reads 146), and competitive alignment 147 between sequence reads 144 that aligned to an oncogenic pathogen reference construct, reference human construct 132, and oncogenic pathogen reference constructs 134;
an oncogenic pathogen identification module 150 that uses alignment data 140 to determine whether the subject is afflicted with an oncogenic pathogen;
an oncogenic pathogen alignment tracking data store 152 for storing sequence alignment counts 156 for individual oncogenic pathogens for test subjects 154; and
an optional patient reporting module 160 for generating reports about the cancer status of a test subject.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. For instance, in some embodiments, sequence alignment data store 136 is integrated in test subject data store 122. Likewise, in some embodiments, rather than having a separate sequence alignment data store 136, the system annotates sequence read entries 128 to indicate the results of the first alignment, second alignment, and/or competitive alignment. For instance, in some embodiments, each entry 128 includes a field for the nucleic acid sequence of the sequence read, a field for the result of alignment against the human reference construct 132 (e.g., whether the sequence read was positively mapped to the human reference construct and/or the location or sequence in the human reference construct that the sequence read was aligned to), a field for the result of alignment against the oncogenic pathogen reference constructs 134 (e.g., whether the sequence read was positively mapped to an oncogenic pathogen reference construct, the identity of the oncogenic pathogen to which the sequence was mapped, and/or the location or sequence in the oncogenic pathogen reference construct that the sequence read was aligned to), and a field for the result of competitive alignment against both the human reference construct 132 and the oncogenic pathogen reference constructs 134 (e.g., the identity of the reference construct to which the sequence read was positively mapped to and/or the location or sequence in the reference construct that the sequence read was aligned to).

In some implementations, the non-persistent memory 111 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 may retrieve all or a portion of such data when needed.

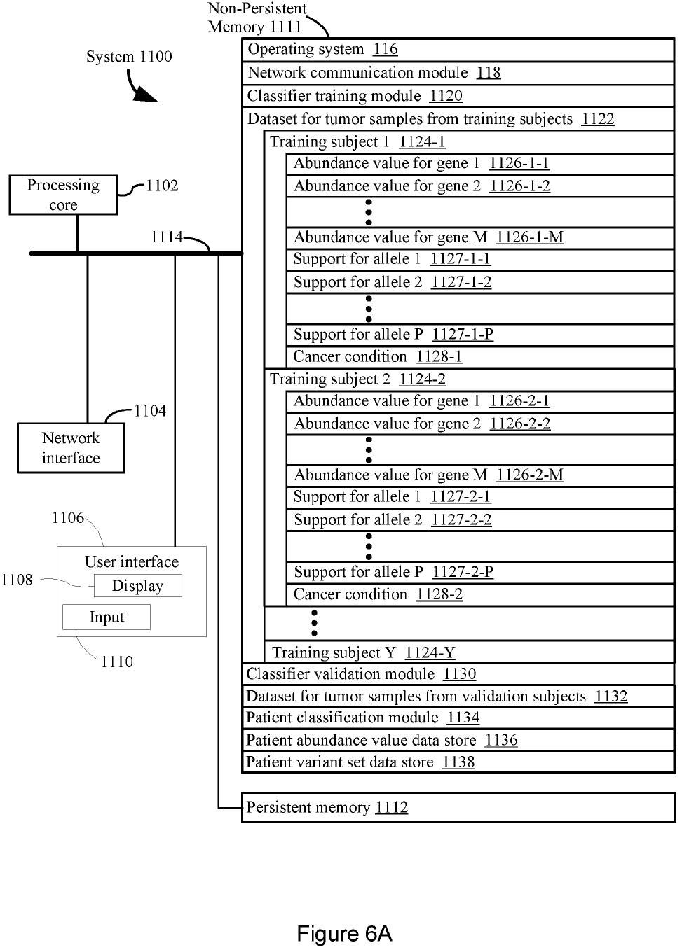

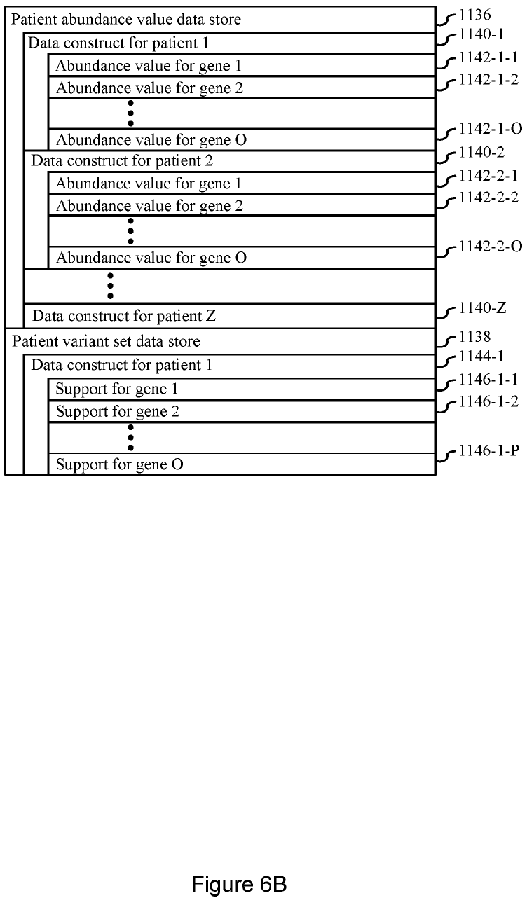

RNA sequencing-based pathogen detection – FIG. 6 is a block diagram illustrating a system 1100 in accordance with some implementations. The device 1100 in some implementations includes one or more processing units CPU(s) 1102 (also referred to as processors), one or more network interfaces 1104, a user interface 1106, a non-persistent memory 1111, a persistent memory 1112, and one or more communication buses 1114 for interconnecting these components. The one or more communication buses 1114 optionally include circuitry (sometimes called a chipset) that interconnects and controls communications between system components. The non-persistent memory 1111 typically includes high-speed random access memory, such as DRAM, SRAM, DDR RAM, ROM, EEPROM, flash memory, whereas the persistent memory 1112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 1112 optionally includes one or more storage devices remotely located from the CPU(s) 1102. The persistent memory 1112, and the non-volatile memory device(s) within the non-persistent memory 1112, comprise non-transitory computer readable storage medium. In some implementations, the non-persistent memory 1111 or alternatively the non-transitory computer readable storage medium stores the following programs, modules and data structures, or a subset thereof, sometimes in conjunction with the persistent memory 1112:
an optional operating system 1116, which includes procedures for handling various basic system services and for performing hardware dependent tasks;
an optional network communication module (or instructions) 1118 for connecting the system 1100 with other devices and/or a communication network 1105;
an optional classifier training module 1120 for training classifiers that distinguish a first cancer condition, associated with an oncogenic pathogen infection, from a second cancer condition, that is not associated with an oncogenic pathogen infection;
an optional data store for datasets for tumor samples from training subjects 1122 including expression data from one or more training subjects 1124, where the expression data includes a plurality of abundance data for each of a plurality of genes 1126, support for a plurality of variant alleles for each of one or more genes 1127, and a cancer condition 1128;
an optional classifier validation module 1130 for validating classifiers that distinguish a first cancer condition, associated with an oncogenic pathogen infection, from a second cancer condition, that is not associated with an oncogenic pathogen infection;
an optional data store for datasets for tumor samples from validation subjects including expression data from one or more training subjects, where the expression data includes a plurality of abundance data for each of a plurality of genes and a cancer condition;
an optional patient classification module 1134 for classifying a cancer in a patient as either a first cancer condition, associated with an oncogenic pathogen infection, or a second cancer condition, that is not associated with an oncogenic pathogen infection, using a classifier, e.g., as trained using classifier training module 1120;
an optional data store for data constructs for cancer patients 1136 including expression data from one or more cancer patients 1140, where the expression data includes a plurality of abundance data for each of a plurality of genes 1142; and
an optional data store for data constructs for cancer patients 1138 including variant allele data from one or more cancer patients 1144, where the variant allele data includes a plurality of support for variant alleles for each of one or more genes 1146.

In various implementations, one or more of the above identified elements are stored in one or more of the previously mentioned memory devices, and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations. In some implementations, the non-persistent memory 1111 optionally stores a subset of the modules and data structures identified above. Furthermore, in some embodiments, the memory stores additional modules and data structures not described above. In some embodiments, one or more of the above identified elements is stored in a computer system, other than that of visualization system 1100, that is addressable by visualization system 1100 so that visualization system 1100 may retrieve all or a portion of such data when needed.

Although FIGS. 1 and 6 depict a “system 100” or “system 1100,” the figures are intended more as functional description of the various features which may be present in one or more computer systems than as a structural schematic of the implementations described herein. In practice, and as recognized by those of ordinary skill in the art, items shown separately could be combined and some items could be separated. Moreover, although FIGS. 1 and 6 depict certain data and modules in non-persistent memory 111 and 1111, some or all of these data and modules may be in persistent memory 112 and 1112.

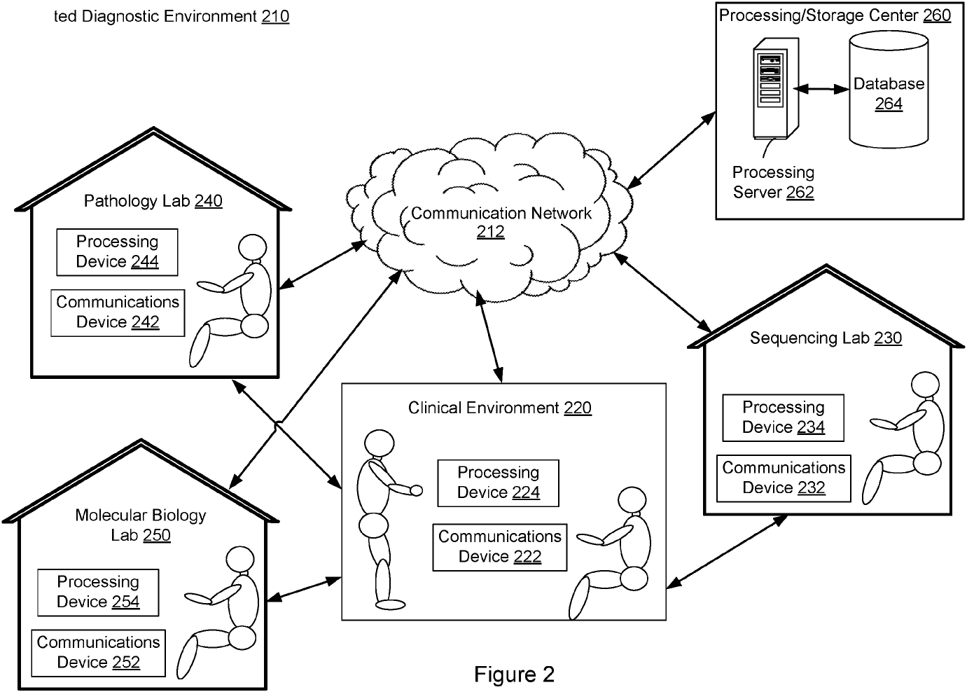

For instance, as depicted in FIG. 2, in some embodiments the method is performed across a distributed diagnostic environment 210, e.g., connected via communication network 212. In some embodiments, one or more biological sample, e.g., one or more tumor biopsy or control sample, is collected from a subject in clinical environment 220, e.g., a doctor’s office, hospital, or medical clinic. In some embodiments, a portion of the sample is processed within the clinical environment using a processing device 224, e.g., a nucleic acid sequencer for obtaining sequencing data, a microscope for obtaining pathology data, a mass spectrometer for obtaining proteomic data, etc. In some embodiments, the biological sample or a portion of the biological sample is sent to one or more external environments, e.g., sequencing lab 230, pathology lab 240, and molecular biology lab 250, each of which includes a processing device 234, 244, and 254, respectively, to generate biological data about the subject. Each environment includes a communications device 222, 232, 242, and 252, respectively, for communicating biological data about the subject to a processing server 262 and/or database 264, which may be located in yet another environment, e.g., processing/storage center 260. Thus, in some embodiments, different portions of the systems and methods described herein are fulfilled by different processing devices located in different physical environments.

### DNA Sequencing-Based Oncogenic Pathogen Detection

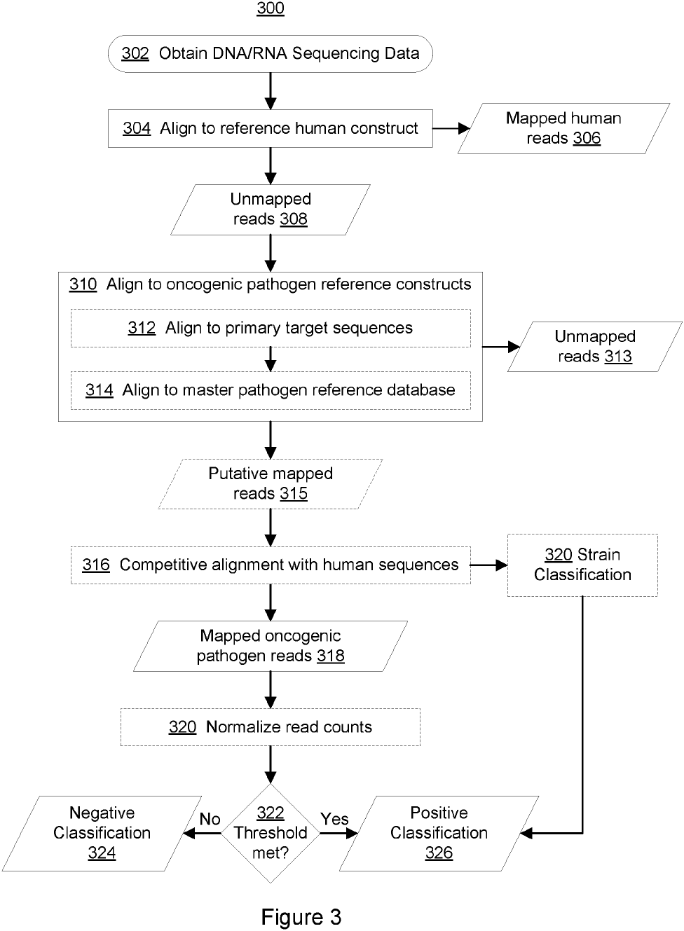

While systems in accordance with the present disclosure has been disclosed with reference to FIGS. 1A, 1B, and 2, an overview of methods in accordance with the present disclosure are provided in conjunction with FIG. 3. In block 302, a dataset containing DNA and/or RNA sequencing data 124 from a sample from a test subject is obtained, e.g., a tumor biopsy collected at clinical environment 220. In some embodiments, the sequencing data is generated at a second environment, e.g., sequencing lab 230, using a different processing device 234, e.g., a nucleic acid sequencer, than subsequent processing steps, e.g., performed at processing server 262. In some embodiments, the sequencing is performed after enriching nucleic acids derived from a plurality of predetermined target sequences, e.g., human genes and/or non-coding sequences associated with cancer. In some embodiments, the enrichment is achieved by binding the nucleic acids from the biological sample to a set of hybridization probes having sequences with homology to the predetermined target sequences or the complement thereof. In some embodiments, the set of hybridization probes also includes a subset of probes with sequences that are complementary to sequences from one or more selected oncogenic pathogens.

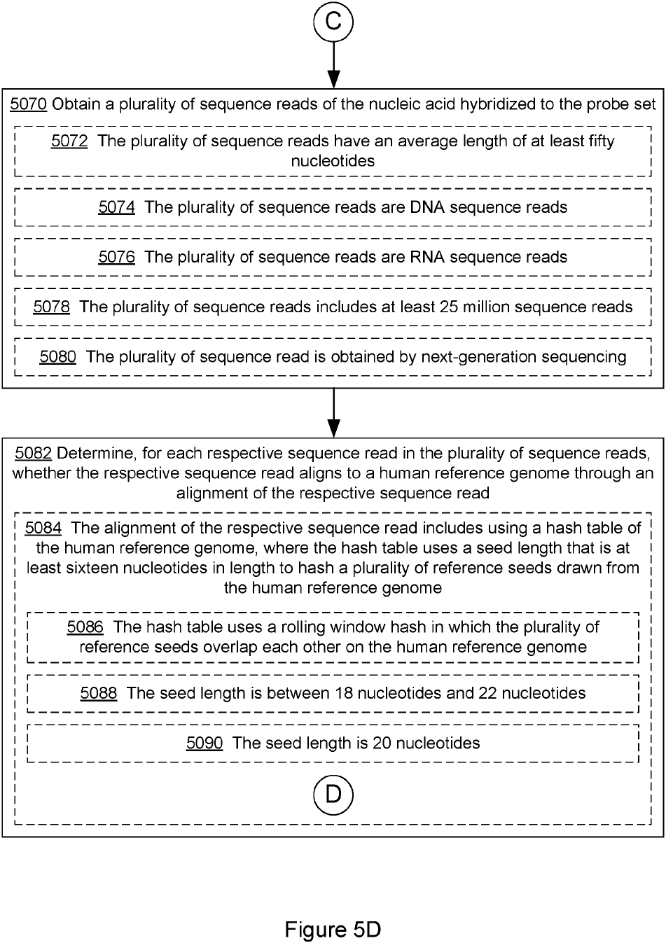

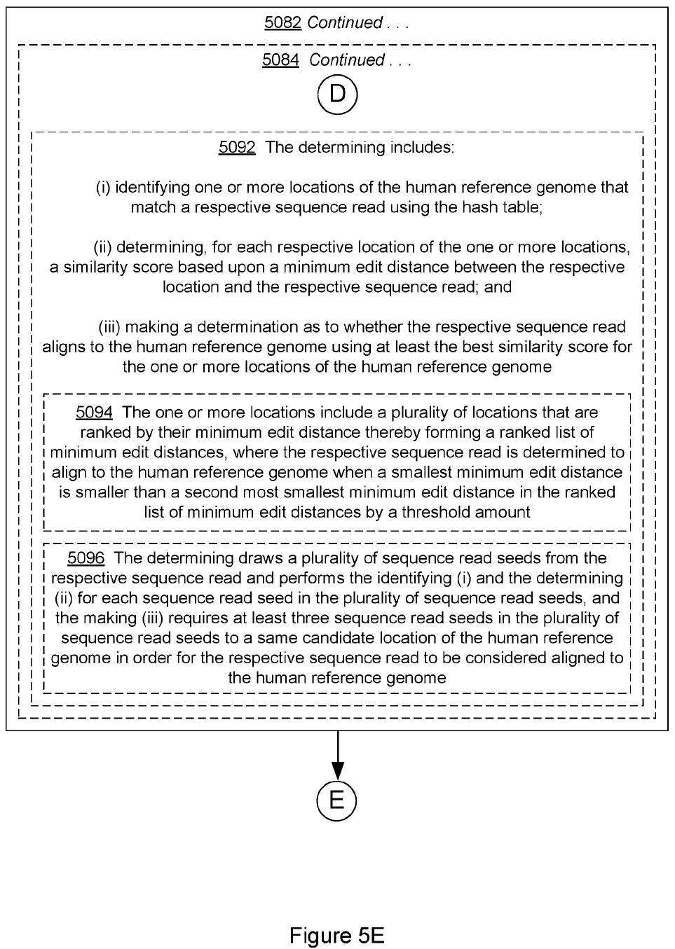

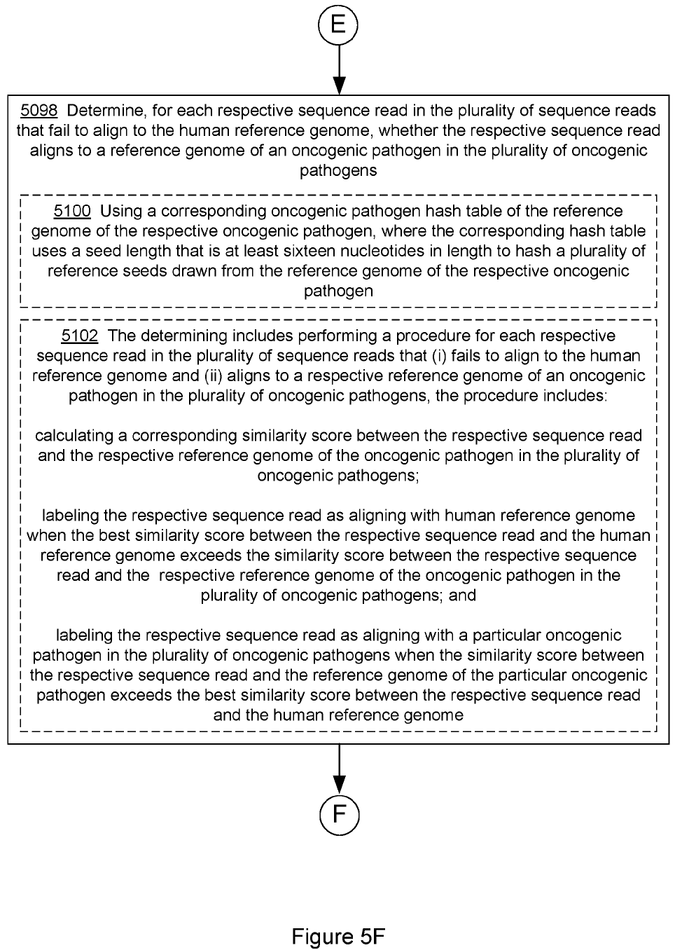

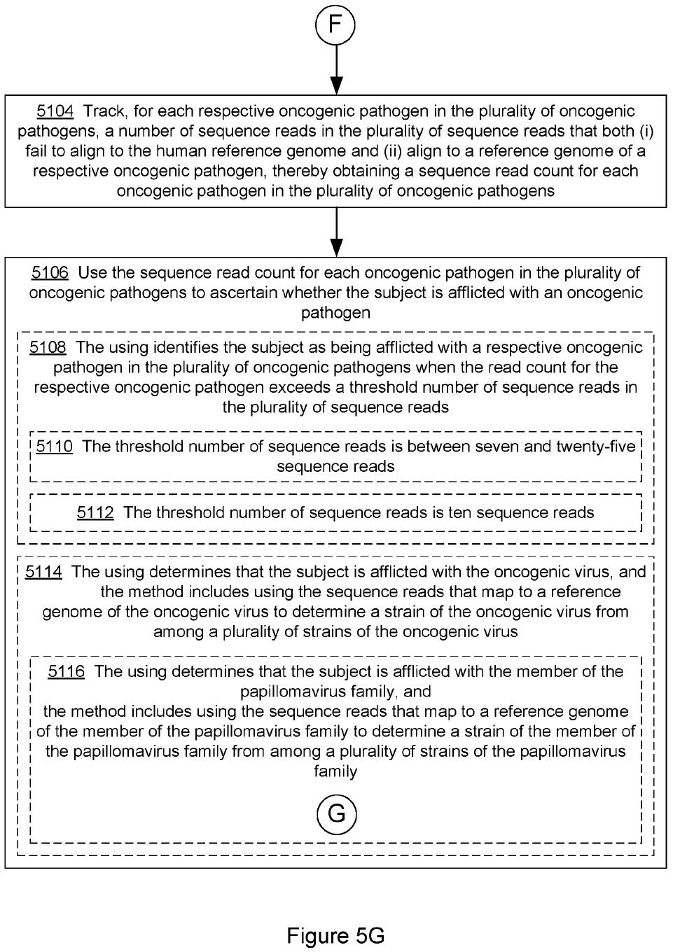

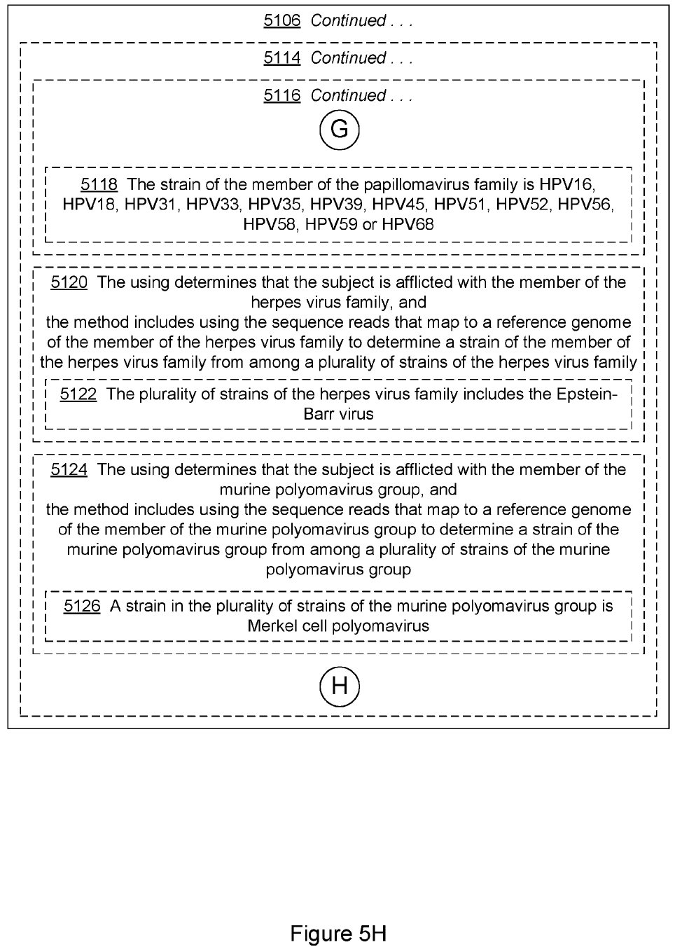

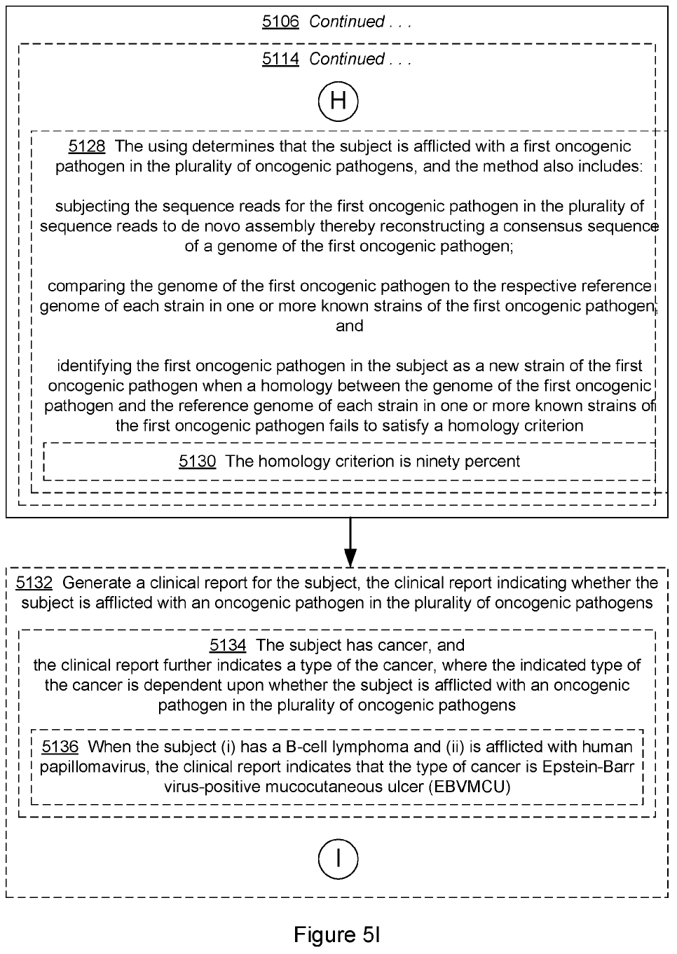

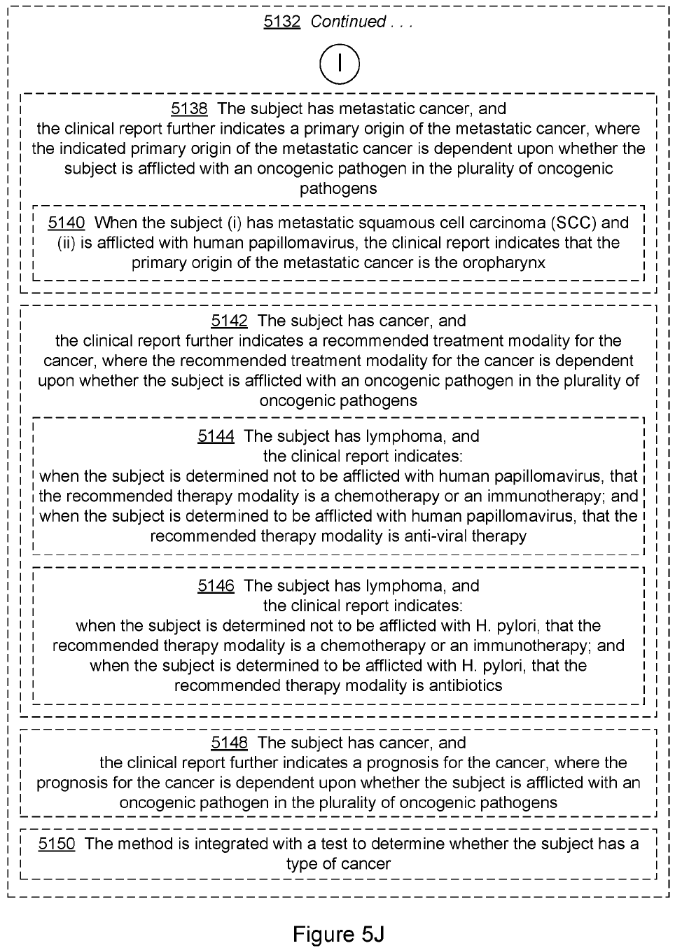

Many of the embodiments described below, in conjunction with FIGS. 3 and 5, relate to analyses performed using sequencing data from the genome and/or exome of a cancer patient, e.g., obtained from a sample of the cancerous tissue in the patient. Generally, these embodiments are independent and, thus, not reliant upon any particular expression data generation methods, e.g., sequencing, hybridization, and/or qPCR methodologies. However, in some embodiments, the methods described below include one or more steps of generating the sequencing data.

In block 304, individual sequence reads 128, in electronic form, are aligned against a reference human data construct 132, e.g., a reference human genome or reference human exome, using sequence alignment module 130. In some embodiments, the alignment is performed with an index-based alignment algorithm, e.g., a hash-based sequence alignment algorithm. The index-based alignment algorithm runs more quickly than a conventional local alignment algorithm, but generally with lower performance such that, overall, fewer sequence reads will be correctly mapped to a position within the reference human data construct. There are two advantages to the use of an index-based alignment algorithm at this step: first, the alignment is less computationally burdensome, resulting in a quicker and more efficient computational process, and second, fewer sequence reads with significant identity to both the human reference construct and to an oncogenic pathogen reference construct are aligned to the human reference construct and, thus, removed from the data set prior to subsequent alignment to the oncogenic pathogen reference construct, resulting in improved sensitivity for the detection of oncogenic pathogen-derived sequence reads. The result of block 304 is a partitioning of the sequencing data 124 into a first subset of sequence reads 306 (e.g., aligned sequences 140) that definitively map to the human reference construct and a second subset of sequence reads 308 (e.g., unaligned sequences 142) that do not definitively map to the human reference construct.

In block 310, individual sequence reads 142 in the second subset of sequence reads 308 are aligned against a plurality of oncogenic pathogen reference constructs 134, e.g., reference genomes or reference exomes for a plurality of oncogenic pathogens. In some embodiments, the alignment is performed with an index-based alignment algorithm, e.g., a hash-based sequence alignment algorithm. The index-based alignment algorithm runs more quickly and efficiently than a conventional local alignment algorithm.

In some embodiments, where both the alignment against the human reference construct and the alignment against the oncogenic pathogen reference constructs are performed using the same sequence alignment algorithm, a parameter of the sequence alignment algorithm is defined more stringently during the alignment against the human reference construct than during the alignment against the oncogenic pathogen reference constructs. In this fashion, more sequences that align to both the human reference construct and one or more of the oncogenic pathogen reference constructs are identified because (i) they are not removed from the analysis by being assigned to subset 306 of sequence reads that definitively align to the human reference construct, and are therefore not aligned against the oncogenic pathogen reference constructs, and (ii) are identified as aligning to an oncogenic pathogen reference construct because of the lower stringency requirements for assignment of a positive alignment. Subsequently, these sequences can be further queried to determine whether they align better to the human reference construct or the oncogenic pathogen reference construct, as described below.

In other embodiments, sequence reads 306 that are identified as aligning to the human reference construct (e.g., aligned sequence reads 140) are also aligned against one or more of the oncogenic pathogen reference constructs 134. In some embodiments, sequence reads 306 are aligned against all of the oncogenic pathogen reference constructs in the same fashion that unmapped sequence reads 308 are aligned to the oncogenic pathogen reference constructs. In some embodiments, sequence reads 306 that are identified as aligning to the human reference construct are aligned against just a subset of oncogenic pathogen reference constructs, e.g., primary oncogenic pathogen reference constructs, in the same fashion that unmapped sequence reads 308 are aligned to the primary target oncogenic pathogen reference constructs. In some embodiments, sequence reads 306 are aligned against all of a subset of the oncogenic pathogen reference constructs using a different alignment algorithm, e.g., one that runs faster than, but may be less sensitive than, the alignment algorithm used to align unmapped sequence reads 308 against the oncogenic pathogen reference constructs.

In some embodiments, alignment of sequence reads 308 against the plurality of oncogenic pathogen reference constructs is performed in two steps. First, each of the sequence reads is aligned (312) against a sub-plurality of reference constructs for one of more primary target oncogenic pathogens. Second, each sequence read that did not align to any one of the sub-plurality of reference constructs is aligned against the other oncogenic pathogen reference constructs in the plurality of oncogenic pathogen reference constructs. In some embodiments, where a hybridization probe set is used to enrich target nucleic acids from the biological sample, the hybridization probe set includes a sub-set of probes complementary to nucleic acid sequences from the one or more primary target oncogenic pathogens, e.g., but does not include probes complementary to other oncogenic pathogens. The result of block 310 is partitioning of sequence reads 308 into a third subset of sequence reads 313 that do not map to either the human reference construct or any of the oncogenic pathogen reference constructs (e.g., unaligned sequence reads 146) and a fourth subset of sequence reads that align to at least one of the oncogenic pathogen reference constructs (e.g., aligned sequence reads 144).

In some embodiments, sequence reads that are putatively mapped to at least one of the oncogenic pathogen reference constructs (e.g., aligned sequence reads 144) are then competitively aligned against the at least one oncogenic pathogen reference construct 134 and the human reference construct 132, to determine which reference construct each sequence read aligns to better. In some embodiments, the competitive alignment is performed with a local sequence alignment algorithm, e.g., which aligns each nucleotide, rather than an index-based alignment algorithm. Although local sequence alignment algorithms require more computational resources, the algorithm is more sensitive and therefore performs better than an index-based sequence alignment algorithm on average. Advantageously, because the majority of the original sequencing data has been removed by assignment to mapped human reads 306 (e.g., aligned sequence reads 140) or unmapped reads 313 (e.g., unaligned sequence reads 146), e.g., using less computationally taxing alignment algorithms, this process facilitates high confidence assignment of oncogenic pathogen sequence reads 318 more quickly than if all of the sequencing data was aligned to the oncogenic pathogen reference constructs, providing a more efficient computational process (e.g., the set of aligned sequence reads 144 is much smaller than the set of all sequence reads 128 for a subject).

The method includes tracking sequence reads identified as aligning to one or more oncogenic pathogen reference constructs. The number of sequence reads that are finally aligned to each oncogenic pathogen following the competitive alignment (316), e.g., mapped oncogenic pathogen reads 318, are counted, e.g., using oncogenic pathogen identification module and stored in oncogenic pathogen alignment tracking data store 152, as counts 156 for each pathogen. In some embodiments, as depicted in box 320, sequence counts 156 for the alignment data are normalized, e.g., to account for pull-down, amplification, and/or sequencing bias (e.g., mappability, GC bias etc.). See, for example, Schwartz et al., 2011, “Detection and Removal of Biases in the Analysis of Next-Generation Sequencing Reads,” PLoS ONE 6(1): e16685.doi:10.1371/journal.pone.0016685; and Benjamini and Speed, 2012 “Summarizing and correcting the GC content bias in high-throughput sequencing,” Nucleic Acids Research 40(10) e72, each of which is hereby incorporated by reference.

A determination (322) is then made as to whether a threshold number of sequences aligning to each of the one or more oncogenic pathogen reference constructs have been identified. If a threshold number sequences aligning to a respective oncogenic pathogen reference construct have been identified, the subject is classified (326) as afflicted by the respective oncogenic pathogen. If a threshold number sequences aligning to a respective oncogenic pathogen reference construct have not been identified, the subject is classified (324) as not afflicted by the respective oncogenic pathogen.

In some embodiments, the classification for each respective oncogenic pathogen is used to inform classification of the subject’s cancer, e.g., to determine a type of cancer, a primary origin of the cancer, a prognosis for the cancer, and/or a recommendation for treating the cancer. Non-limiting examples of oncogenic pathogens that are known to be associated with specific cancers are shown below in Table 1. For additional information on known associations between oncogenic pathogens and cancers see, for example, Flora and Bonanni, 2011, “The prevention of infection-associated cancers,” Carcinogenesis 32(6), pp. 787-795, which is hereby incorporated by reference.

TABLE 1

Pathogen infections associated with cancer in humans

| | |
|---|---|
| PATHOGEN (COLUMN 1) | ASSOCIATED CANCER (COLUMN 2) |
| Hepatitis virus - HBV | Hepatocellular carcinoma (HCC) |
| Hepatitis virus - HCV | Hepatocellular carcinoma (HCC) |
| Papillomaviruses (HPV) – (e.g., Alpha HPV types 16, 18, 26, 30, 31, 33, 34, 35, 39, 45, 51, 52, 53, 56, 58, 59, 66, 67, 68, 69, 70, 73, 82, 85, and 97) | Cervical cancer, Head and Neck Squamous Cell Carcinoma |
| Papillomaviruses (HPV) – (e.g., Beta HPV types 5 and 8) | Skin Cancer |
| Polyomaviruses – (e.g., JCV) | CNS tumors |
| Polyomaviruses – (e.g., MCV) | Skin cancer |
| Polyomaviruses – (e.g., SV40) | Malignant mesothelioma |
| Herpesviruses (e.g., EBV or HHV4) | Burkitt’s lymphoma, sinonasal angiocentric T-cell lymphoma, immunosuppressor-related non-Hodgkin’s lymphoma, Hodgkin’s lymphoma, nasopharyngeal carcinoma, Gastric Carcinoma |
| Herpesviruses (e.g., KSHV or HHV8) | Kaposi’s sarcoma, primary effusion lymphoma |
| Retroviruses (e.g., HTLV-I) | Adult T-cell leukemia/lymphoma |
| Retroviruses (e.g., HIV-I) | Kaposi’s sarcoma, non-Hodgkin’s lymphoma, Hodgkin’s lymphoma, cervical cancer, anus cancer, conjunctive cancer |
| Retroviruses (e.g., HIV-2) | Kaposi’s sarcoma, non-Hodgkin’s lymphoma |
| Retroviruses (e.g., HERV-K) | Human breast cancer |
| Retroviruses (e.g., XMRV) | Prostate cancer |
| Helicobacter pylori | Non-cardia gastric cancer, MALT lymphoma |
| Streptococcus bovis | Colorectal cancer |
| Salmonella typhi | Gallbladder cancer |
| Bartonella species | Vascular tumors |
| Human gut microbiome | Colon cancer |
| Clamydophila pneumonia | Lung cancer |
| Schistosoma haematobium | Urinary bladder cancer |
| Schistosoma japonicum | Colorectal and liver cancers |
| Liver fluke (e.g., Opistorchis viverrini, Opistorchis sinensis) | Cholangiocarcinoma |

As used herein, the term “human gut microbiome” refers to all of the microorganisms living in the human digestive tract, a subset of which have been found to be oncogenic. For example, pathogens that have been hypothesized to cause, or are correlated with, colon or colorectal cancers include Sulfidogenic bacteria (e.g. Fusobacterium, Desulfovibrio, and Bilophila wadsworthia), Streptococcus bovis, and Fusobacterium nucleatum. For further information, see, Dahmus et al., 2018, J Gastrointest Oncol., 9(4), pp. 769-77, which is hereby incorporated by reference herein.

In some embodiments, the classification for each respective oncogenic pathogen is used to generate a clinical report that indicates whether the subject is afflicted with an oncogenic pathogen. In some embodiments, the clinical report provides additional information about the subject’s cancer, e.g., a type of cancer, a primary origin of the cancer, a stage of the cancer, a tumor burden for the subject, a prognosis for the subject, a recommended treatment for the cancer, etc. An example of such a clinical report is shown in FIG. 6.

Now that an overview of the disclosed methods has been provided in conjunction with FIG. 3, attention turns to FIGS. 5A through 5J, which provide further details regarding specific implementations of the disclosed methods. Specifically, FIGS. 5A-5J illustrate a flow chart of processes and features for determining whether a subject is afflicted with an oncogenic pathogen, in accordance with some embodiments of the present disclosure.

In some embodiments, method 5000 is performed, at least partially, at a computer system (e.g., computer system 100 in FIG. 1) having one or more processors, and memory storing one or more programs for execution by the one or more processors for determining whether a subject is afflicted with an oncogenic pathogen. Some operations in method 5000 are, optionally, combined and/or the order of some operations is, optionally, changed. In some embodiments, various portions of method 5000 are performed by cancer classification module 120, sequence alignment module 130, oncogenic pathogen identification module 150, or patient reporting module 160.

### Nucleic Acid Isolation

Although method 5000 includes steps of obtaining nucleic acids from a biological sample from a subject and hybridizing the nucleic acid to a probe set, in some embodiments the disclosed methods begin by obtaining sequence data from the isolated nucleic acids, as illustrated in FIG. 3. For example, in some embodiments, the first step of method 5000 is to obtain a plurality of sequence reads 126 from nucleic acids isolated from a biological sample from the subject, e.g., by sequencing isolated the nucleic acids or by receiving sequence reads, in electronic form, previously generated from the isolated nucleic acids, which may or may not have been enriched through hybridization to a probe set, as disclosed herein. Accordingly, in some embodiments, the sequence reads are obtained by whole genome or whole exome sequencing methodology. In other embodiments, the sequence reads are obtained by target-based sequencing methodologies.

In some embodiments, method 5000 includes obtaining (5002) an amount of nucleic acid from a biological sample of the subject, where the amount of nucleic acid includes nucleic acid from the subject and potentially nucleic acid from at least one oncogenic pathogen in a plurality of oncogenic pathogens. In some embodiments, the plurality of oncogenic pathogens includes one or more members of the papillomavirus family, one or more members of the herpes virus family, and/or one or more members of the murine polyomavirus group (5010).

Generally, the biological sample of the subject is a biopsy, e.g., a sample of cancerous tissue from the subject. Methods for obtaining samples of cancerous tissue are known in the art, and are dependent upon the type of cancer being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, the biological sample is a solid biopsy (5030). In some embodiments, the solid biopsy is a macro-dissected formalin fixed paraffin embedded (FFPE) tissue section (5032). In some embodiments, the biological sample comprises blood or saliva (5034). In some embodiments, the subject has cancer (5036).

Similarly, methods for isolating nucleic acids from biological samples are known in the art, and are dependent upon the type of nucleic acid being isolated, e.g., DNA or RNA, and the type of sample from which the nucleic acids are being isolated. For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. For example, acid guanidinium thiocyanate-phenol-chloroform extraction (see, for example, Chomczynski and Sacchi, 2006, Nat Protoc, 1(2):581-85, which is hereby incorporated by reference herein), and silica bead/glass fiber adsorption (see, for example, Poeckh, T. et al., 2008, Anal Biochem., 373(2):253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed.

In some embodiments, the plurality of oncogenic pathogens includes one or more oncogenic viruses (5004). For example, in some embodiments, the plurality of oncogenic pathogens includes 1, 2, 3, 4, 5, 6, 7, 8, 9, 10, 15, 20, 25, 30, 40, 50, or more oncogenic viruses. In some embodiments, each oncogenic pathogen in the plurality of oncogenic pathogens is an oncogenic virus (5006). In some embodiments, an oncogenic pathogen in the plurality of oncogenic pathogens is an oncogenic virus listed in Table 1 (5008). For further information on oncogenic viruses see, for example, de Flora, 2011, Carcinogenesis 32:787-95, which is incorporated by reference herein.

In some embodiments, the plurality of oncogenic pathogens includes a member of the papillomavirus family of viruses. Papillomaviruses are non-enveloped DNA viruses, for which several hundred species have been identified see, for example, Van Doorslaer K. et al., J Gen Virol., 99(8):989-990 (2018), which is incorporated by reference herein. In some embodiments, the member of the papillomavirus family is human papillomavirus (HPV) (5012). In some embodiments, the human papillomavirus is HPV16, HPV18, HPV31, HPV33, HPV35, HPV39, HPV45, HPV51, HPV52, HPV56, HPV58, HPV59 or HPV68 (5014). For more information on the various species of human papillomavirus see, for example, Chouhy D. et al., 2013, J Gen Virol., 94(11):2480-88, which is incorporated by reference herein. In some embodiments, the one or more human papillomaviruses includes HPV16 or HPV18 (5016), both of which are known to be associated with human cancers see, for example, Saraiya M. et al., 2015, Natl Cancer Inst., 107(6), which is incorporated by reference herein.

In some embodiments, the plurality of oncogenic pathogens includes a member of the herpes virus family. Herpesviridae are enveloped, monopartite, double-stranded, linear DNA viruses; see, for example, Mettenleiter et al., 2008, “Animal Viruses: Molecular Biology,” Caister Academic Press, Chapter 9 “Molecular Biology of Animal Herpesviruses,” which is incorporated by reference herein. Nine species of herpesviridae are known to infect humans, including herpes simplex viruses 1 and 2 (HSV-1 and HSV-2), varicella-zoster virus (VZV), Epstein-Barr virus (EBV), human cytomegalovirus (HCMV), human herpesvirus 6A and 6B (HHV-6A and HHV-6B), human herpesvirus 7 (HHV-7), and Kaposi’s sarcoma-associated herpesvirus (KSHV). Many of these species have been associated with human cancers. For example, Epstein-Barr virus (EBV) has been linked to several human neoplasms, including Burkitt’s lymphoma, sinonasal angiocentric T-cell lymphoma, immunosuppressor-related non-Hodgkin’s lymphoma, Hodgkin’s lymphoma, nasopharyngeal carcinoma, Gastric Carcinoma; see, for example, Rezk SA et al., Hum Pathol., 79:18-41 (2018), which is incorporated by reference herein. Human cytomegalovirus (HCMV) has been associated with oncomodulation and oncogenesis in various cancers, including glioma, colorectal cancer, prostate cancer, breast cancer, mucoepidermoid carcinoma, medulloblastoma, and neuroblastoma; see, for example, Herbein G., Viruses, 10(8):408 (2018), which is incorporated by reference herein. Kaposi’s sarcoma-associated herpesvirus (KSHV) has been associated with Kaposi’s sarcoma and primary effusion lymphoma; see, for example, Goncalves PH et al., Curr Opin HIV AIDS, 12(1):47-56 (2017), which is incorporated herein by reference. Additionally, some studies have suggested a link between human herpesvirus 6A and 6B (HHV-6A and HHV-6B) and various cancers, including lymphomas, gliomas, gastrointestinal cancers, cervical cancer, and leukemia; for review see HHV-6 Foundation “HHV-6 & Cancer,” published online. Accordingly, in some embodiments, the one or more members of the herpes virus family includes Epstein-Barr virus (5018). In some embodiments, the member of the herpes virus family is Human cytomegalovirus (HCMV). In some embodiments, the member of the herpes virus family is Kaposi’s sarcoma-associated herpesvirus (KSHV). In some embodiments, the member of the herpes virus family is human herpesvirus 6 (e.g., HHV-6A and/or HHV-6B).

In some embodiments, the plurality of oncogenic pathogens includes a member of the of the polyomavirus family of viruses. Polyomaviruses are non-enveloped, double-stranded, circular DNA viruses; see, for example, Moens et al., 2017, Journal of General Virology, 98:1159-60, which is incorporated by reference herein. Merkel cell polyomavirus (MCPyV), a member of the polyomavirus family, has been associated with Merkel cell carcinomas; see, for example, Rotondo et al., 2017, Clin Cancer Res., 23(14):3929-34, which is incorporated by reference herein. Accordingly, in some embodiments, the one or more member of the polyomavirus family includes Merkel cell polyomavirus (5020).

In some embodiments, the plurality of oncogenic pathogens includes one or more oncogenic bacterium (5022). Several bacteria have been linked to various cancers, including Bacteroides fragilis (colon cancer), Borrelia burgdorferi (MALT lymphoma), Campylobacter jejuni (Immunoproliferative small intestinal disease (IPSID)), Chlamydia pneumonia (Lung MALT lymphoma), Chlamydia trachomatis (Cervical cancer), Chlamydophila psittaci (Ocular/adnexal lymphoma), Clostridiumssp. (Colon cancer), Helicobacter bilis, (gallbladder and biliary tract cancers), Helicobacter bizzozeronii (Gastric MALT lymphoma), Helicobacter felis (Gastric MALT lymphoma), Helicobacter heilmannii (Gastric MALT lymphoma), Helicobacter hepaticus (Biliary cancer), Helicobacter pylori (Stomach cancer), Helicobacter salomonis (Gastric MALT lymphoma), Helicobacter suis (Gastric MALT lymphoma), Mycoplasmaspp. (Stomach, colon, ovarian, and lung cancers), Neisseria gonorrhoeae (Bladder and prostate cancer), Cutibacterium acnes (Bladder and prostate cancer), Salmonella enterica serovar Paratyphi (Biliary cancer), Salmonella enterica serovar Typhimurium (Biliary cancer), and Treponema pallidum (Bladder and prostate cancer). See, for example, Sinkovics, 2012, Int. J. Oncol. 40(2):305-49; Chang and Parsonnet, 2010, J, Clin. Microbiol. Rev. 23(4):837-57, which are incorporated by reference herein. In some embodiments, the oncogenic bacterium is an oncogenic bacterium listed in Table 1 (5024).

In some embodiments, the plurality of oncogenic pathogens includes one or more oncogenic trematodes (5026). Several trematodes have been linked to various cancers, including Schistosoma haematobium (bladder cancer), Opisthorchis viverrini (bile duct cancer), and Clonorchis sinensis (bile duct cancer). See, for example, Bouvard et al., 2009, Lancet Oncol. 10(4):321-22. In some embodiments, the oncogenic trematode is an oncogenic trematode listed in Table 1.

Yet other types of oncogenic pathogens have been identified, including protozoan parasites (e.g., Toxoplasma gondii, Cryptosporidium parvum, Trichomonas vaginalis, Theileria, and Plasmodium falciparum), tapeworms (e.g., Echinococcus granulosus and Taenia solium), liver flukes (e.g., Fasciola gigantica and Platynosomum fastosum), and roundworms (e.g., Strongyloides stercoralis, Heterakis gallinarum, and Trichuris muris). For more information on other oncogenic parasites see, for example, Machicado and Marcos, 2016, Int. J. Cancer 138(12):2915-21, which is incorporated by reference herein.

### Enrichment of Target Sequences

In some embodiments, the methods described herein include enriching nucleic acids isolated from the biological sample for target sequences associated with cancer classification. Advantageously, enriching for target sequences prior to sequencing the nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample. Accordingly, in some embodiments, method 5000 includes hybridizing (5038) the amount of nucleic acid to a probe set, where the probe set includes a plurality of nucleic acid probes for a plurality of human genomic loci and a respective set of nucleic acid probes for genomic loci of each respective oncogenic pathogen in the plurality of oncogenic pathogens.

Generally, the probes include DNA, RNA, or a modified nucleic acid structure with a base sequence that is complementary to a locus of interest. Accordingly, when the probe is designed to hybridize to an mRNA molecule isolated from the biological sample, the probe will include a nucleic acid sequence that is complementary to the coding strand of the gene from which the transcript originated, i.e., the probe will include an antisense sequence of the gene. However, when the probe is designed to hybridize to a loci in a gDNA molecule or cDNA molecule, the probe can contain either a sequence that is complementary to either strand, because the molecules in the gDNA or cDNA library are double stranded. In some embodiments, each probe in the plurality of probes includes a nucleic acid sequence that is identical or complementary to at least 15 consecutive bases of a locus of interest. In some embodiments, each probe in the plurality of probes includes a nucleic acid sequence that is identical or complementary to at least 20, 25, 30, 40, 50, 75, 100, 150, 200, or more consecutive bases of a locus of interest.

In some embodiments, the probes include additional nucleic acid sequences that do not share any homology to the loci of interest. For example, in some embodiments, the probes also include nucleic acid sequences containing an identifier sequence, e.g., a unique molecular identifier (UMI), e.g., that is unique to a particular sample or subject. Examples of identifier sequences are described, for example, in Kivioja et al., 2011, Nat. Methods 9(1), pp. 72-74 and Islam et al., 2014, Nat. Methods 11(2), pp. 163-66, which are incorporated by reference herein. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to nucleic acid molecule that is complementary to the loci of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest. In some embodiments, the methods described herein include amplifying (5060) the nucleic acids that bound to the probe set prior to further analysis, e.g., sequencing. Methods for amplifying nucleic acids, e.g., by PCR, are well known in the art.

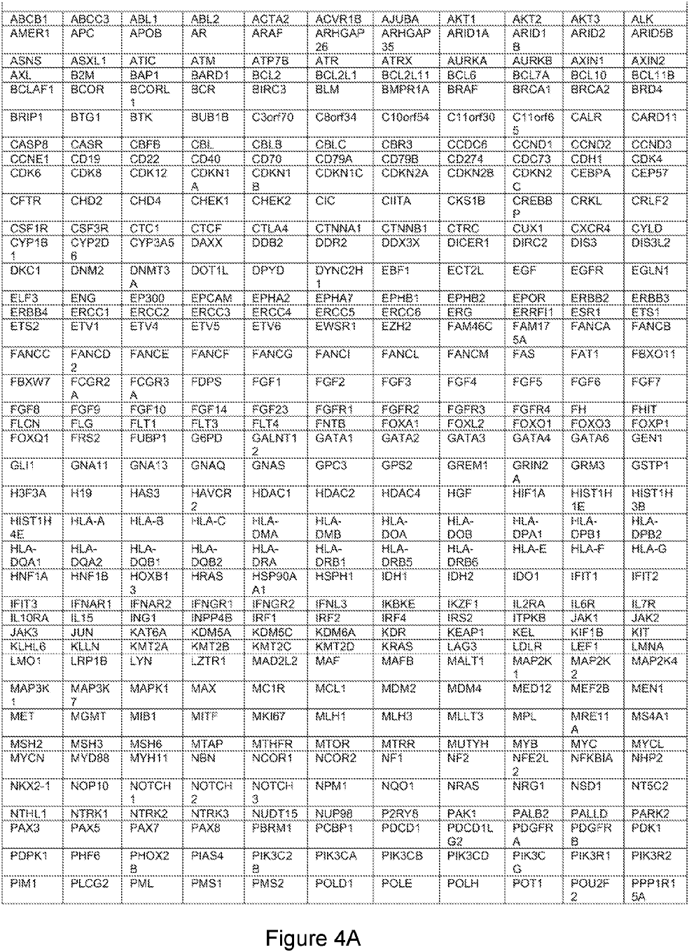

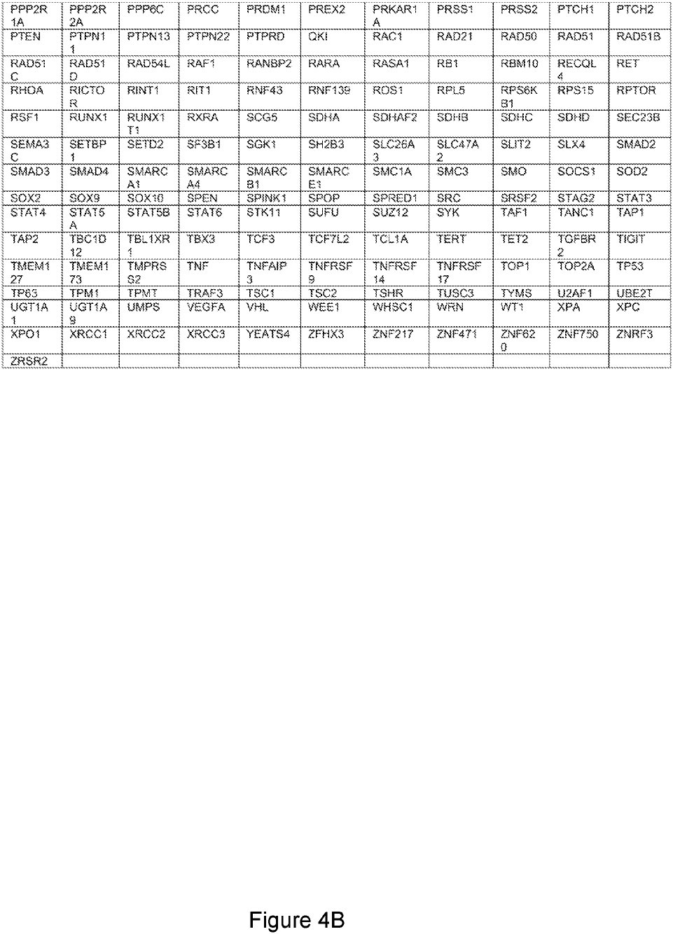

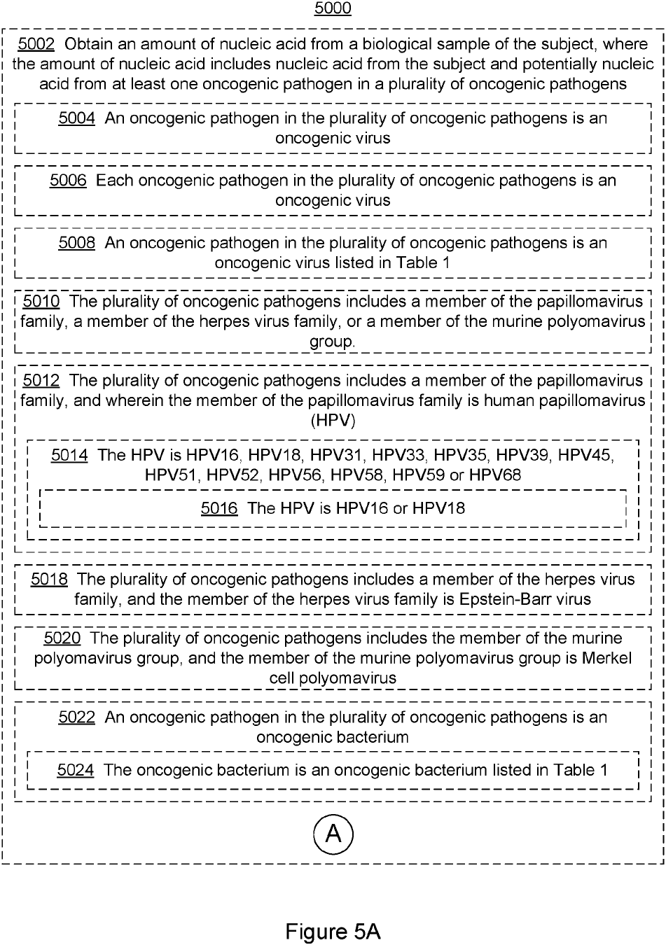

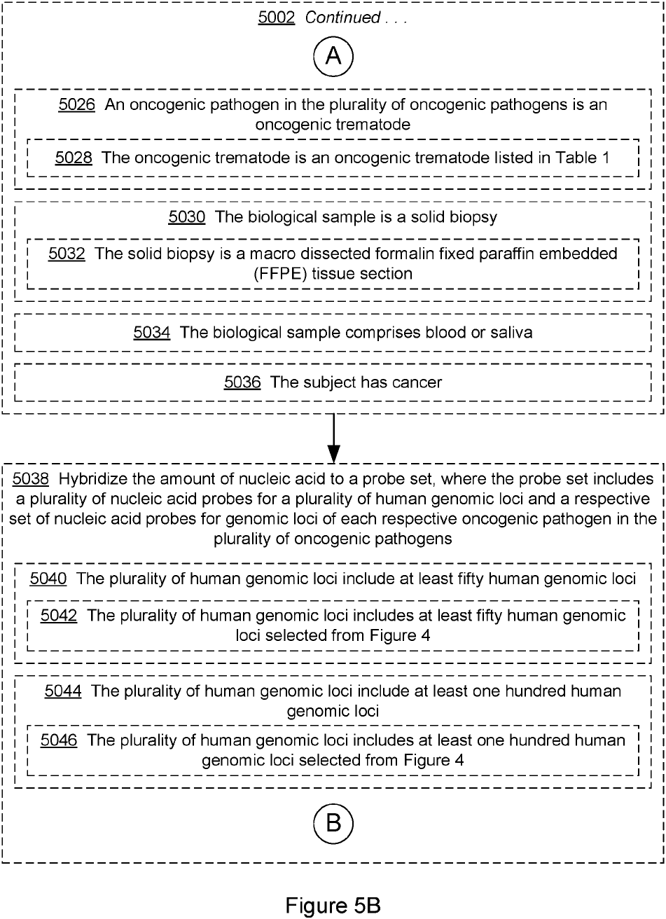

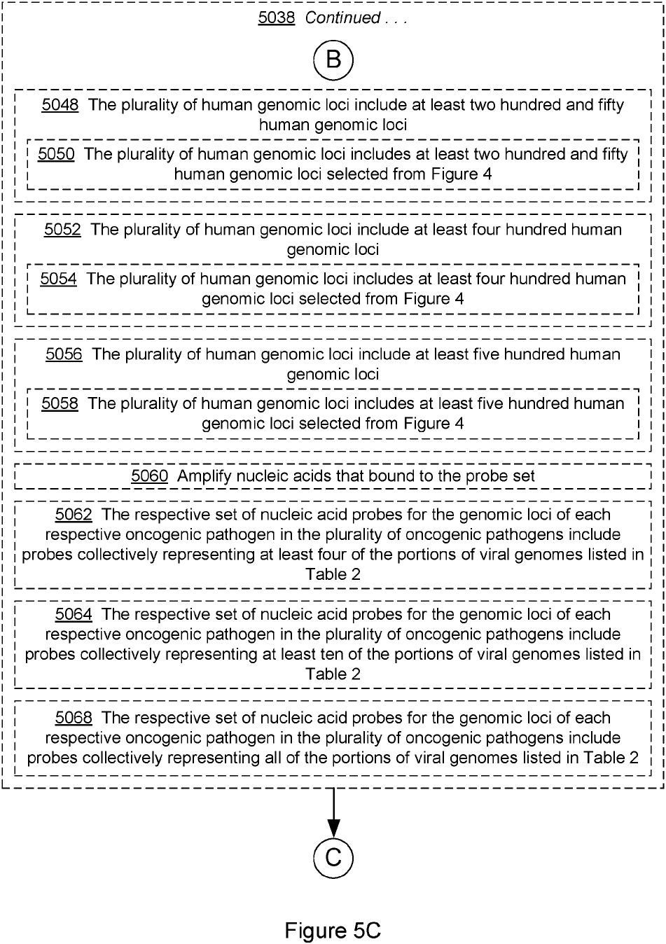

The human genomic loci can include gene loci, e.g., exon or intron loci, as well as non-coding loci, e.g., regulatory loci and other non-coding loci, which have been found to be associated with cancer. In some embodiments, the plurality of human genomic loci include at least 25, 50, 100, 150, 200, 250, 300, 350, 400, 500, 750, 1000, 2500, 5000, or more human genomic loci. In one embodiment, the plurality of human genomic loci include at least fifty human genomic loci (5040). In one embodiment, the plurality of human genomic loci includes at least fifty human genomic loci selected from FIG. 4 (5042). In one embodiment, the plurality of human genomic loci include at least one hundred human genomic loci (5044). In one embodiment, the plurality of human genomic loci includes at least one hundred human genomic loci selected from FIG. 4 (5046). In one embodiment, the plurality of human genomic loci include at least two hundred and fifty human genomic loci (5048). In one embodiment, the plurality of human genomic loci includes at least two hundred and fifty human genomic loci selected from FIG. 4 (5050). In one embodiment, the plurality of human genomic loci include at least four hundred human genomic loci (5052). In one embodiment, the plurality of human genomic loci includes at least four hundred human genomic loci selected from FIG. 4 (5054). In one embodiment, the plurality of human genomic loci include at least five hundred human genomic loci (5056). In one embodiment, the plurality of human genomic loci includes at least five hundred human genomic loci selected from FIG. 4 (5058).

In some embodiments, the probe set includes probes to genomic loci in one or more oncogenic pathogens selected from alphapapillomavirus (APV), gammaherpesvirus (GHV), HBV genotype A, HPV16, HPV18, HPV33, EBV, MCPyV, Bacteroides fragilis, Helicobacter pylori, Serratia marcescens, and Chlamydia trachomatis. Examples of loci in genes encoded by each of these oncogenic pathogens are provided in Table 2. In some embodiments, the probe set includes probes to at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60, at least 70, at least 80, at least 90, at least 100, at least 125, at least 150, at least 175, or of the loci listed in Table 2. In some embodiments, the respective set of nucleic acid probes for the genomic loci of each respective oncogenic pathogen in the plurality of oncogenic pathogens include probes collectively representing at least four of the portions of viral and/or bacterial genomes listed in Table 2 (5062). In some embodiments, the respective set of nucleic acid probes for the genomic loci of each respective oncogenic pathogen in the plurality of oncogenic pathogens include probes collectively representing at least ten of the portions of viral and/or bacterial genomes listed in Table 2 (5064). In some embodiments, the respective set of nucleic acid probes for the genomic loci of each respective oncogenic pathogen in the plurality of oncogenic pathogens include probes collectively representing all of the portions of viral genomes listed in Table 2. A portion or all of the probes listed may be used for DNA-sequencing and/or for RNA-sequencing. In one example, probes targeting alphapapillomavirus, HBV, HPV16, HPV18, HPV33, EBV (or human gammaherpesvirus 4), human gammaherpesvirus 8, MCPyV, Bacteroides fragilis, Helicobacter pylori, Serratia marcescens, and Chlamydia trachomatis are used for DNA-sequencing and probes targeting alphapapillomavirus, gammaherpesvirus, HBV, HPV16, HPV18, HPV33, EBV, MCPyV, Bacteroides fragilis, Helicobacter pylori, and Chlamydia trachomatis are used for RNA-sequencing.

TABLE 2

Example target loci in the genomes of oncogenic pathogens associated with cancer in humans

| PATHOGEN REFERENCE GENOME BUILD | PATHOGEN | START POSITION | END POSITION | GENE NAME |
|---|---|---|---|---|
| NC_001526.4 | HPV16 | 7125 | 7601 | E6 |
| NC_001526.4 | HPV16 | 7604 | 7900 | E7 |
| NCBI:taxid333760 | HPV16 | 0 | 120 | NCBI:taxid333760 |
| NCBI:taxid333760 | HPV16 | 119 | 239 | NCBI:taxid333760 |
| NCBI:taxid333760 | HPV16 | 238 | 358 | NCBI:taxid333760 |
| NCBI:taxid333760 | HPV16 | 357 | 477 | NCBI:taxid333760 |
| NCBI:taxid333760 | HPV16 | 0 | 120 | NCBI:taxid333760 |
| NCBI:taxid333760 | HPV16 | 89 | 209 | NCBI:taxid333760 |
| NCBI:taxid333760 | HPV16 | 178 | 298 | NCBI:taxid333760 |
| NC_001357.1 | HPV18 | 105 | 581 | E6 |
| NC_001357.1 | HPV18 | 590 | 907 | E7 |
| M12732 | HPV33 | 109 | 558 | E6 |
| M12732 | HPV33 | 573 | 866 | E7 |
| NCBI:taxid10586 | HPV33 | 0 | 120 | |
| NCBI:taxid10586 | HPV33 | 110 | 230 | |
| NCBI:taxid10586 | HPV33 | 220 | 340 | |
| NCBI:taxid10586 | HPV33 | 330 | 450 | |
| NCBI:taxid10586 | HPV33 | 0 | 120 | |
| NCBI:taxid10586 | HPV33 | 87 | 207 | |
| NCBI:taxid10586 | HPV33 | 174 | 294 | |
| NC_007605.1 | EBV | 55189 | 55361 | EBNA-1 |
| NC_007605.1 | EBV | 36098 | 37739 | EBNA-2 |
| NC_007605.1 | EBV | 166461 | 168507 | LMP-1 |
| NC_007605.1 | EBV | 166103 | 166458 | LMP-2 |
| NC_007605.1 | EBV | 58 | 272 | LMP-2 |
| NC_007605.1 | EBV | 55189 | 55361 | EBNA-1 |
| NC_007605.1 | EBV | 36098 | 37739 | EBNA-2 |
| NC_007605.1 | EBV | 360 | 458 | LMP-2 |
| NC_007605.1 | EBV | 540 | 788 | LMP-2 |
| NC_007605.1 | EBV | 871 | 951 | LMP-2 |
| NC_007605.1 | EBV | 1026 | 1196 | LMP-2 |
| NC_007605.1 | EBV | 1280 | 1495 | LMP-2 |
| NC_007605.1 | EBV | 1574 | 1680 | LMP-2 |
| NCBI:taxid10376 | EBV | 0 | 120 | |
| NCBI:taxid10376 | EBV | 52 | 172 | |
| NCBI:taxid10376 | EBV | 0 | 120 | |
| NCBI:taxid10376 | EBV | 96 | 216 | |
| NCBI:taxid10376 | EBV | 0 | 120 | |
| NCBI:taxid10376 | EBV | 2 | 122 | |
| NCBI:taxid10376 | EBV | 0 | 120 | |
| NCBI:taxid10376 | EBV | 118 | 238 | |
| NCBI:taxid10376 | EBV | 236 | 356 | |
| NCBI:taxid10376 | EBV | 0 | 120 | |
| NCBI:taxid10376 | EBV | 113 | 233 | |
| NCBI:taxid10376 | EBV | 226 | 346 | |
| NCBI:taxid10376 | EBV | 339 | 459 | |
| NCBI:taxid10376 | EBV | 452 | 572 | |
| NCBI:taxid10376 | EBV | 565 | 685 | |
| NCBI:taxid10376 | EBV | 678 | 798 | |
| NCBI:taxid10376 | EBV | 791 | 911 | |
| NCBI:taxid10376 | EBV | 904 | 1024 | |
| NCBI:taxid10376 | EBV | 1017 | 1137 | |
| NCBI:taxid10376 | EBV | 1130 | 1250 | |
| NCBI:taxid10376 | EBV | 1243 | 1363 | |
| NCBI:taxid10376 | EBV | 1356 | 1476 | |
| NCBI:taxid10376 | EBV | 1469 | 1589 | |
| NCBI:taxid10376 | EBV | 1582 | 1702 | |
| NCBI:taxid10376 | EBV | 1695 | 1815 | |
| NCBI:taxid10376 | EBV | 1808 | 1928 | |
| NCBI:taxid10376 | EBV | 1921 | 2041 | |
| NCBI:taxid10376 | EBV | 0 | 120 | |
| NCBI:taxid10376 | EBV | 2 | 122 | |
| NCBI:taxid10376 | EBV | 0 | 120 | |
| NCBI:taxid10376 | EBV | 117 | 237 | |
| NCBI:taxid10376 | EBV | 234 | 354 | |
| NCBI:taxid10376 | EBV | 351 | 471 | |
| NCBI:taxid10376 | EBV | 468 | 588 | |
| NCBI:taxid10376 | EBV | 585 | 705 | |
| NCBI:taxid10376 | EBV | 702 | 822 | |
| NCBI:taxid10376 | EBV | 819 | 939 | |
| NCBI:taxid10376 | EBV | 936 | 1056 | |
| NCBI:taxid10376 | EBV | 1053 | 1173 | |
| NCBI:taxid10376 | EBV | 1170 | 1290 | |
| NCBI:taxid10376 | EBV | 1287 | 1407 | |
| NCBI:taxid10376 | EBV | 1404 | 1524 | |
| NCBI:taxid10376 | EBV | 1521 | 1641 | |
| NCBI:taxid10376 | EBV | 0 | 120 | |
| NCBI:taxid10376 | EBV | 64 | 184 | |
| NCBI:taxid10376 | EBV | 128 | 248 | |
| NCBI:taxid37296 | GHV8 | 3159 | 3279 | |
| NCBI:taxid37296 | GHV8 | 0 | 120 | |
| NCBI:taxid37296 | GHV8 | 117 | 237 | |
| NCBI:taxid37296 | GHV8 | 234 | 354 | |
| NCBI:taxid37296 | GHV8 | 351 | 471 | |
| NCBI:taxid37296 | GHV8 | 468 | 588 | |
| NCBI:taxid37296 | GHV8 | 585 | 705 | |
| NCBI:taxid37296 | GHV8 | 702 | 822 | |
| NCBI:taxid37296 | GHV8 | 819 | 939 | |
| NCBI:taxid37296 | GHV8 | 936 | 1056 | |
| NCBI:taxid37296 | GHV8 | 1053 | 1173 | |
| NCBI:taxid37296 | GHV8 | 1170 | 1290 | |
| NCBI:taxid37296 | GHV8 | 1287 | 1407 | |
| NCBI:taxid37296 | GHV8 | 1404 | 1524 | |
| NCBI:taxid37296 | GHV8 | 1521 | 1641 | |
| NCBI:taxid37296 | GHV8 | 1638 | 1758 | |
| NCBI:taxid37296 | GHV8 | 1755 | 1875 | |
| NCBI:taxid37296 | GHV8 | 1872 | 1992 | |
| NCBI:taxid37296 | GHV8 | 1989 | 2109 | |
| NCBI:taxid37296 | GHV8 | 2106 | 2226 | |
| NCBI:taxid37296 | GHV8 | 2223 | 2343 | |
| NCBI:taxid37296 | GHV8 | 2340 | 2460 | |
| NCBI:taxid37296 | GHV8 | 2457 | 2577 | |
| NCBI:taxid37296 | GHV8 | 2574 | 2694 | |
| NCBI:taxid37296 | GHV8 | 2691 | 2811 | |
| NCBI:taxid37296 | GHV8 | 2808 | 2928 | |
| NCBI:taxid37296 | GHV8 | 2925 | 3045 | |
| NCBI:taxid37296 | GHV8 | 3042 | 3162 | |
| NCBI:taxid37296 | GHV8 | 3276 | 3396 | |
| NCBI:taxid37296 | GHV8 | 3393 | 3513 | |
| NCBI:taxid37296 | GHV8 | 3510 | 3630 | |
| NCBI:taxid37296 | GHV8 | 3627 | 3747 | |
| NCBI:taxid37296 | GHV8 | 3744 | 3864 | |
| NCBI:taxid37296 | GHV8 | 3861 | 3981 | |
| NCBI:taxid37296 | GHV8 | 3978 | 4098 | |
| NCBI:taxid37296 | GHV8 | 4095 | 4215 | |
| NCBI:taxid489450 | HBV | 0 | 120 | |
| NCBI:taxid489450 | HBV | 95 | 215 | |
| NCBI:taxid489450 | HBV | 190 | 310 | |
| NCBI:taxid489450 | HBV | 285 | 405 | |
| NCBI:taxid337042 | APV7 | 0 | 120 | |
| NCBI:taxid337042 | APV7 | 119 | 239 | |
| NCBI:taxid337042 | APV7 | 238 | 358 | |
| NCBI:taxid337042 | APV7 | 357 | 477 | |
| NCBI:taxid337042 | APV7 | 0 | 120 | |
| NCBI:taxid337042 | APV7 | 100 | 220 | |
| NCBI:taxid337042 | APV7 | 200 | 319 | |
| NC_010277.2 | MCPyV | 400 | 1200 | GP1 |
| NC_010277.2 | MCPyV | 5000 | 5200 | GP4 |
| NCBI:taxid493803 | MCPyV | 0 | 120 | |
| NCBI:taxid493803 | MCPyV | 114 | 234 | |
| NCBI:taxid493803 | MCPyV | 228 | 348 | |
| NCBI:taxid493803 | MCPyV | 342 | 462 | |
| NCBI:taxid493803 | MCPyV | 456 | 576 | |
| NCBI:taxid493803 | MCPyV | 570 | 690 | |
| NCBI:taxid493803 | MCPyV | 684 | 801 | |
| NCBI:taxid493803 | MCPyV | 0 | 120 | |
| NCBI:taxid493803 | MCPyV | 81 | 201 | |
| NCBI:txid817 | B. fragilis | 0 | 120 | |
| NCBI:txid817 | B. fragilis | 109 | 229 | |
| NCBI:txid817 | B. fragilis | 218 | 338 | |
| NCBI:txid817 | B. fragilis | 327 | 447 | |
| NCBI:txid817 | B. fragilis | 436 | 556 | |
| NCBI:txid817 | B. fragilis | 545 | 665 | |
| NCBI:txid817 | B. fragilis | 654 | 774 | |
| NCBI:txid817 | B. fragilis | 763 | 883 | |
| NCBI:txid817 | B. fragilis | 872 | 992 | |
| NCBI:txid817 | B. fragilis | 981 | 1101 | |
| NCBI:txid817 | B. fragilis | 1090 | 1209 | |
| NCBI:txid210 | H. pylori | 0 | 120 | |
| NCBI:txid210 | H. pylori | 97 | 217 | |
| NCBI:txid210 | H. pylori | 194 | 314 | |
| NCBI:txid210 | H. pylori | 291 | 411 | |
| NCBI:txid210 | H. pylori | 388 | 508 | |
| NCBI:txid210 | H. pylori | 485 | 603 | |
| NCBI:txid210 | H. pylori | 0 | 120 | |
| NCBI:txid210 | H. pylori | 116 | 236 | |
| NCBI:txid210 | H. pylori | 232 | 352 | |
| NCBI:txid210 | H. pylori | 348 | 468 | |
| NCBI:txid210 | H. pylori | 0 | 120 | |
| NCBI:txid210 | H. pylori | 109 | 229 | |
| NCBI:txid210 | H. pylori | 218 | 338 | |
| NCBI:txid210 | H. pylori | 327 | 447 | |
| NCBI:txid210 | H. pylori | 436 | 555 | |
| NCBI:txid615 | S. marcescens | 0 | 120 | |
| NCBI:txid615 | S. marcescens | 109 | 229 | |
| NCBI:txid615 | S. marcescens | 218 | 338 | |
| NCBI:txid615 | S. marcescens | 327 | 447 | |
| NCBI:txid615 | S. marcescens | 436 | 556 | |
| NCBI:txid615 | S. marcescens | 545 | 665 | |
| NCBI:txid615 | S. marcescens | 654 | 774 | |
| NCBI:txid615 | S. marcescens | 763 | 883 | |
| NCBI:txid813 | C. trachomatis | 0 | 120 | |
| NCBI:txid813 | C. trachomatis | 117 | 237 | |
| NCBI:txid813 | C. trachomatis | 234 | 354 | |
| NCBI:txid813 | C. trachomatis | 351 | 471 | |
| NCBI:txid813 | C. trachomatis | 468 | 588 | |
| NCBI:txid813 | C. trachomatis | 585 | 705 | |
| NCBI:txid813 | C. trachomatis | 702 | 822 | |

### Nucleic Acid Sequencing

The methods described herein include obtaining a plurality of sequence reads, in electronic form, of nucleic acids isolated from the biological sample from the subject. In some embodiments, the sequence reads are obtained from a nucleic acid sample that has been enriched for target sequences, as described above. Advantageously, as described above, sequencing a nucleic acid sample that has been enriched for target nucleic acids, rather than all nucleic acids isolated from a biological sample, significantly reduces the average time and cost of the sequencing reaction. Accordingly, in some embodiments, method 5000 includes obtaining (5070) a plurality of sequence reads (e.g., sequence reads 128) of the nucleic acid hybridized to the probe set, e.g., as described above.

In some embodiments, the sequence reads have an average length of at least fifty nucleotides (5072). In other embodiments, the sequence reads have an average length of at least 25, 30, 35, 40, 45, 50, 60, 70, 80, 90, 100, 150, or more nucleotides.

In some embodiments, the plurality of sequence reads are DNA sequence reads (5074). That is, the nucleic acids isolated from the biological sample are DNA molecules, e.g., genomic DNA (gDNA) molecules or fragments (such as cell-free DNA) thereof.

In some embodiments, the plurality of sequence reads are RNA sequence reads (5076). That is, the nucleic acids isolated from the biological sample are RNA molecules, e.g., mRNA. In some embodiments, RNA sequence reads are obtained directly from the isolated RNA, e.g., by direct RNA sequencing. Methods for direct RNA sequencing are well known in the art. See, for example, Ozsolak et al., 2009, Nature 461:814-18, and Garalde et al., 2018, Nat Methods, 15(3):201-206, which are incorporated by reference herein.

In other embodiments, RNA sequence reads are obtained through a cDNA intermediate. Accordingly, in some embodiments, the isolated RNA is used to create a cDNA library via cDNA synthesis. In some embodiments, both for direct RNA sequencing and prior to cDNA library construction, the isolated RNA is first enriched for a desired type of RNA (e.g., mRNA) or species (e.g., specific mRNA transcripts), prior to cDNA library construction.

Methods of enriching for desired RNA molecules are also well known in the art. For example, mRNA molecules can be enriched, e.g., relative to other RNA molecules in a total RNA preparation, using oligo-dT affinity techniques (see, for example, Rio et al., 2010, Cold Spring Harb Protoc., 2010(7), which is incorporated by reference herein). Specific mRNA transcripts can also be isolated, e.g., using hybridization probes that specifically bind to one or more mRNA sequences of interest.

cDNA library construction from isolated mRNAs is also well known in the art. In some embodiments, cDNA library construction is performed by first-strand DNA synthesis from the isolated mRNA using a reverse transcriptase, followed by second-strand synthesis using a DNA polymerase. Example methods for cDNA synthesis are described in McConnell and Watson, 1986, FEBS Lett. 195(1-2), pp. 199-202; Lin and Ying, 2003, Methods Mol Biol. 221, pp. 129-143, and Oh et al., 2003, Exp Mol Med. 35(6), pp. 586-90, which are incorporated by reference herein.

Methods for mRNA sequencing are well known in the art. In some embodiments, the mRNA sequencing is performed by whole exome sequencing (WES). Generally, WES is performed by isolating RNA from a tissue sample, optionally selecting for desired sequences and/or depleting unwanted RNA molecules, generating a cDNA library, and then sequencing the cDNA library, for example, using next generation sequencing (NGS) techniques. For a review of the use of whole exome sequencing techniques in cancer diagnosis, see, Serratì et al., 2016, Onco Targets Ther. 9, pp. 7355-7365, which is incorporated by reference herein.

RNA-Seq is a methodology used for RNA profiling based on next-generation sequencing that enables the measurement and comparison of gene expression patterns across a plurality of subjects. In some embodiments, millions of short strings, called ‘sequence reads,’ are generated from sequencing random positions of cDNA prepared from the input RNAs that are obtained from tumor tissue of a subject. These reads can then be computationally mapped on a reference genome to reveal a ‘transcriptional map’, where the number of sequence reads aligned to each gene gives a measure of its level of expression (e.g., abundance). Next-generation sequencing is disclosed in Shendure, 2008, “Next-generation DNA sequencing,” Nat. Biotechnology 26, pp. 1135-1145, which is incorporated by reference herein. RNA-Seq is disclosed in Nagalakshmi et al., 2008, “The transcriptional landscape of the yeast genome defined by RNA sequencing,” Science 320, pp. 1344-1349; and Finotell and Camillo, 2014, “Measuring differential gene expression with RNA-seq: challenges and strategies for data analysis,” Briefings in Functional Genomics 14(2), pp. 130-142, which are incorporated by reference herein. Briefly, RNA molecules isolated from a biological sample are initially fragmented and reverse-transcribed into complementary DNAs (cDNAs). The obtained cDNAs are then amplified and subjected to next-generation DNA sequencing (NGS). In principle, any NGS technology can be used for RNA-Seq. In some embodiments, the Illumina sequencer (see the Internet at illumina.com) is used. See, Wang et al., 2009, “RNA-Seq: a revolutionary tool for transcriptomics,” Nat Rev Genet., 10(1):57-63, which is incorporated by reference herein. The millions of short reads generated for each such sample are then mapped on a reference genome and the number of reads aligned to each gene, called ‘counts’, gives a digital measure of gene expression levels in the sample under investigation.

Methods for next generation sequencing, which can be used for either DNA or RNA sequencing, are well known in the art. These include sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

### Sequence Alignment to a Reference Human Genome

The methods for detecting oncogenic pathogens described herein proceed through a computational subtractive process in which sequences that definitively align to a human reference genome are identified and removed from the dataset before the remaining sequence reads are aligned against oncogenic pathogen reference constructs (e.g., as illustrated in steps 304 and 310 in FIG. 3). See, for example, Naccache et al., 2014, Genome Res. 24(7):1180-92; Greninger et al., 2010, PLoS One, 5(10):e13381; Kostic et al., 2011, Nat Biotechnol. 29(5):393-96; MacConaill and Meyerson, 2008, Nat Genet., 40(4):380-82; and Zhao et al., 2013, PLoS One, 8(10):e78470, which are incorporated by reference herein. In this fashion, the computational burden of aligning sequence reads against a plurality of reference constructs is significantly reduced by removing many of the sequence reads. For example, as reported in Examples 2, 3, and 4, alignment of sequence reads generated from three cancer biopsies removed more than 99.5% of the sequence reads in all three cases, and more than 99.8% of the sequence reads in two of the cases. Accordingly, method 5000 includes determining (5082), for each respective sequence read in the plurality of sequence reads, whether the respective sequence read aligns to a human reference genome (e.g., reference human construct 132) through an alignment of the respective sequence read (e.g., using sequence alignment module 130).

In some embodiments, an index-based alignment algorithm is used to decrease the computational time needed to align the sequence reads to the human reference genome. Index-based algorithms construct auxiliary data structures for either or both the read sequences or the reference sequence, and use these structures, which are less complex than the raw sequence, when searching for matches between the read sequences and the reference sequence. Three examples of index-based alignment algorithms are (i) algorithms that use hash tables, (ii) algorithms that are based on suffix trees, and (iii) algorithms based on merge sorting. See, for example, Li and Homer, 2010, Brief Bioinform. 11(5):473-83, which is incorporated by reference herein. Such algorithms are used to exclude large parts of the human reference genome from the expensive dynamic programming comparison used to align a sequence read to the human genome. See, Canzar and Stazberg, 2018, “Short Read Mapping: An Algorithmic Tour,” Proc IEEE Inst. Electr Electron Eng., 105(3), 436-458, which is hereby incorporated by reference.

In one embodiment, the alignment (5082) of the sequence reads against the human reference genome uses a hash-based algorithm. For instance, in some embodiments sequence reads are mapped to the human reference genome using a hash-based algorithm and then aligned using a dynamic programming algorithm. Hash-based algorithms rely on generation of a hash table index of the reference sequence (e.g., a human reference genome), based on k-mers of a particular seed length of the sequence. Query sequences (e.g., sequence reads) are then broken into k-mers of the same length, and the algorithm uses the hash table index to identify regions in the reference sequence that share multiple k-mers with a query sequence. See, for example, Lee WP et al., 2014 PLoS One, 9(3):e90581. Examples of hash-based alignment algorithms include BLAST, MAQ, ZOOM, RMAP, CloudBurst, Eland, mrFAST/mrsFAST, SHRiMP, MOM, MOSAIK, PASS, ProbeMatch, SOAP, SRmapper, and STAMPY. Accordingly, in some embodiments, the alignment of the respective sequence read includes (5084) using a hash table of the human reference genome, where the hash table uses a seed length that is at least sixteen nucleotides in length to hash a plurality of reference seeds drawn from the human reference genome. In some embodiments, the hash table uses a seed length that is from 10 nucleotides to 30 nucleotides in length. In some embodiments, the hash table uses a seed length that is from 15 nucleotides to 25 nucleotides in length. In some embodiments, the seed length is between 18 nucleotides and 22 nucleotides (5088). In some embodiments, the seed length is 20 nucleotides (5090). In yet other embodiments, the hash table uses a seed length that is at least 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the hash table uses a rolling window hash, in which the plurality of reference seeds overlap each other on the human reference genome (5086).

Hash-based mapping algorithms require less computation time to identify possible alignments of a sequence read to a reference genome than global alignment algorithms, because the algorithm does not search for each nucleotide individually. However, this can result in the identification of several putative mappings for the sequence read in the reference genome. Accordingly, the system then determines which, if any, of the putative mappings represents a true alignment with the sequence read (e.g., using a dynamic programming algorithm as disclosed in Canzar and Stazberg, 2018, “Short Read Mapping: An Algorithmic Tour,” Proc IEEE Inst. Electr Electron Eng., 105(3), 436-458, which is hereby incorporated by reference). Accordingly, in some embodiments, the alignment (5082) of the sequence reads against the human reference genome includes (i) identifying one or more locations of the human reference genome that match a respective sequence read (mappings) using the hash table, (ii) determining, for each respective location of the one or more locations, a similarity score based upon a minimum edit distance between the respective location and the respective sequence read (e.g., using a dynamic programming algorithm), and (iii) making a determination as to whether the respective sequence read aligns to the human reference genome using at least the best similarity score for the one or more locations of the human reference genome (5092).

In some embodiments, the determination as to whether the sequence read aligns to any particular locus in the reference genome is done by ranking the putative matches to the sequence read and determining whether the highest ranked alignment is significantly better than the other putative matches in order for a positive match to be assigned. In some embodiments, the one or more (putatively matched) locations (in the reference genome) include a plurality of locations that are ranked by their minimum edit distance thereby forming a ranked list of minimum edit distances, where the respective sequence read is determined to align to the human reference genome when a smallest minimum edit distance is smaller than a second most smallest minimum edit distance in the ranked list of minimum edit distances by a threshold amount (5094). Minimal editing distance is the minimum number of operations (insertions, deletions and substitutions) required to convert one string to another. Methods for determining minimal editing distance are known in the art. For example, see, Mantaci S. et al., Int. J. of Approximate Reasoning, 47:109-24, which is incorporated by reference herein.

In some embodiments, minimum similarity standards are required in order for the system to positively match the sequence read to any locus in the reference genome when using a hash-based alignment algorithm. For instance, in some embodiments, a minimal number of seeds derived from the sequence read must match within a particular locus in the reference genome, ensuring that the putative alignment represents alignment of the entire sequence read, as opposed to just a portion of the sequence read, e.g., corresponding to a single seed length of sequence. Accordingly, in some embodiments, the determining (5082) draws a plurality of sequence read seeds from the respective sequence read and performs the identifying (i; 5092) and the determining (ii; 5092) for each sequence read seed in the plurality of sequence read seeds, and the making (iii; 5092) requires at least three sequence read seeds in the plurality of sequence read seeds to a same candidate location of the human reference genome in order for the respective sequence read to be considered aligned to the human reference genome.

In some embodiments, the alignment (5082) of the sequence reads against the human reference genome uses an algorithm based on suffix trees or a suffix array. Examples of these types of algorithms include MUMmer, MUMmeGPU, Vmatch, PacBio Aligner, Bowtie, Bowtie 2, BWA, and BWA-SW. See for example, Langmead Salzberg, 2012, “Fast gapped-read alignment with Bowtie 2,” Nature Methods 9(4):357-359, which is hereby incorporated by reference.

In other embodiments, the alignment (5082) of the sequence reads against the human reference genome uses an algorithm based on merge sorting. Examples of these types of algorithms include Slider and SliderII.

In some embodiments, the alignment of sequence reads against the human reference genome uses SARUMAN, GPU-RMAP, BarraCUDA, SOAP3, SOAP3-dp, CUSHAW, CUSHAW2-GPU, Burrows-Wheeler transform algorithm, a hashing algorithm, pigeonhole, MAQ, RMAP, SOAP, Hobbes, ZOOM, FastHASH, RazerS, RazerS 3, BFAST SEME, SHRiMP, BWT-SW, BWA, Botie, BLASR, Bowtie 2, BWA-SW, GEM, or SOAP2. For further discussion of these alignment algorithms, see Canzar and Stazberg, 2018, “Short Read Mapping: An Algorithmic Tour,” Proc IEEE Inst. Electr Electron Eng., 105(3), 436-458, which is hereby incorporated by reference.

### Sequence Alignment to a Reference Oncogenic Pathogen Construct

As illustrated in FIG. 3, the alignments of the sequence reads against the human reference genome, as described above, results in the identification of two subsets of sequence reads: those that are identified as mapping to the human reference genome 306 (e.g., aligned sequence reads 140) and those that are not identified as mapping to the human reference genome 308 (e.g., unaligned sequence reads 142). Using the computational subtractive process, those sequence reads that were mapped to the human reference genome 306 (e.g., aligned sequence reads 140) are not used in the next step in the identification process, e.g., they are removed from the working set of sequence reads from which oncogenic pathogen sequences are identified. Thus, in the next step of method 5000, the remaining sequence reads 308 (those reads that were not mapped to the human reference genome; e.g., unaligned sequence reads 142) are aligned against one or more oncogenic pathogen reference constructs 134, e.g., partial or complete reference genomes and or exomes, for a plurality of oncogenic pathogens (e.g., as illustrated in step 310 of FIG. 3; e.g., using sequence alignment module 130). Accordingly, in some embodiments, method 5000 includes determining (5098), for each respective sequence read in the plurality of sequence reads that fail to align to the human reference genome (e.g., subset 308), whether the respective sequence read aligns to a reference genome of an oncogenic pathogen in the plurality of oncogenic pathogens.

Publicly accessible databases of microbial and viral genomes are known to those of skill in the art. For instance, the National Center for Biotechnology Information (NCBI) curates publicly accessible databases of microbial genomes, including archaea genomes and bacterial genomes. Likewise, the NCBI also curates publicly accessible databases of viral databases. In some embodiments, a publically-accessible genome database, such as an NCBI database, is used for identifying sequence reads originating from oncogenic pathogens in the sequence reads that were not mapped to the human reference genome (e.g., unaligned sequence reads 142 as shown in FIG. 1B and/or unmapped reads 308 as shown in FIG. 3). In some embodiments, the genome database includes genomic sequences from non-oncogenic pathogens in addition to genomic sequences from oncogenic pathogens, such as the NCBI databases. In other embodiments, the genome database includes only genomic sequences from oncogenic pathogens.

In some embodiments, the set of pathogenic genomic constructs against which the unmapped sequence reads are aligned includes at least 10 pathogen genomes. In some embodiments, the set of pathogenic genomic constructs against which the unmapped sequence reads are aligned includes at least 100 pathogen genomes. In some embodiments, the set of pathogenic genomic constructs against which the unmapped sequence reads are aligned includes at least 1000 pathogen genomes. In some embodiments, the set of pathogenic genomic constructs against which the unmapped sequence reads are aligned includes at least 10,000 pathogen genomes. In some embodiments, the set of pathogenic genomic constructs against which the unmapped sequence reads are aligned includes at least 100,000 pathogen genomes. In some embodiments, the set of pathogenic genomic constructs against which the unmapped sequence reads are aligned includes at least 1,000,000 pathogen genomes. In some embodiments, the set of pathogenic genomic constructs against which the unmapped sequence reads are aligned includes from 10 pathogen genomes to 2,000,000 pathogen genomes. In some embodiments, the set of pathogenic genomic constructs against which the unmapped sequence reads are aligned includes from 100 pathogen genomes to 2,000,000 pathogen genomes. In some embodiments, the set of pathogenic genomic constructs against which the unmapped sequence reads are aligned includes from 1000 pathogen genomes to 2,000,000 pathogen genomes. In some embodiments, the set of pathogenic genomic constructs against which the unmapped sequence reads are aligned includes from 10,000 pathogen genomes to 2,000,000 pathogen genomes. In some embodiments, the set of pathogenic genomic constructs against which the unmapped sequence reads are aligned includes from 100,000 pathogen genomes to 2,000,000 pathogen genomes.

In some embodiments, unmapped sequence reads 308 are first aligned (312) against primary target sequences, e.g., sequences from the genome or exome of an oncogenic pathogen for which a probe was included in the probe set used to enrich nucleic acids isolated from the biological sample from the subject prior to sequencing. In some embodiments, the primary target sequences only include sequences corresponding to the sequences (or complement thereof) of the probes included in the enrichment probe set. In other embodiments, the primary target sequences include whole reference genomes or exomes for the oncogenic pathogens of primary interest.

In some embodiments, after aligning the unmapped sequence reads 308 against the primary target sequences, any remaining sequence reads (e.g., those sequence reads that also did not map to the primary target sequences) are then aligned against a larger database containing reference sequences (e.g., partial or complete reference genomes or exomes, such as the microbial and viral genome databases maintained by the NCBI) for a plurality of other pathogens (e.g., as illustrated in step 314 of FIG. 3). In this fashion, a second computational subtraction step is used, to reduce the number of sequences that are aligned against the larger database. That is, in some embodiments, the device first aligns the sequencing data against a reference genome (e.g., step 304 in FIG. 3) to generate a first set of reads that are mapped to the reference genome (e.g., aligned sequence reads 140 as shown in FIG. 1B and/or mapped reads 306 as shown in FIG. 3). Then, the device aligns the remaining sequence reads (e.g., unaligned sequence reads 140 as shown in FIG. 1B and/or unmapped reads 308 as shown in FIG. 3) to a set of primary target sequences (e.g., step 312 in FIG. 3) to generate a second set of aligned sequence reads that map to a sequence in the genome of a target oncogenic pathogen (e.g., aligned sequence reads 144 as shown in FIG. 1B) and a second set of unaligned sequence reads that do not map to a sequence in the genome of a target oncogenic pathogen (e.g., unaligned sequence reads 146 as shown in FIG. 1B). The device then aligns the second set of unaligned sequence reads against a larger database of oncogenic pathogen genomes (e.g., the microbial and/or viral genome databases maintained by the NCBI) in a third alignment step (e.g., step 314 in FIG. 3), which generates a third set of aligned sequence reads (e.g., aligned sequence reads 148 as shown in FIG. 1B and/or putative mapped reads 315 as shown in FIG. 3). Because the alignment against the larger database requires greater computational time, this second subtractive step improves the efficiency of the process, thereby reducing the computational burden and time required for the method.

In other embodiments, all of unmapped sequence reads 308 are aligned (314) against a database of reference sequences (e.g., partial or complete reference genomes or exomes) that include the plurality of oncogenic pathogens (e.g., as illustrated in step 314 of FIG. 3), without being aligned against a set of primary target sequences. That is, step 312 as shown in FIG. 3 is not performed and aligned sequence reads 144 and unaligned sequence reads 146 as shown in FIG. 1B are not generated.

In some embodiments, in a similar fashion as described above with reference to the alignment of sequence reads against the reference human genome, alignment of the remaining unmapped sequence reads 308 to the database of reference sequences can be sped-up by using an index-based sequence alignment algorithm, e.g., an algorithm that uses hash tables, an algorithm that is based on a suffix tree, or an algorithm based on merge sorting.

In one embodiment, the alignment (5098) of the sequence reads against reference constructs for the oncogenic pathogens uses a hash-based alignment algorithm. Accordingly, in some embodiments, method 5000 includes using (5100) a corresponding oncogenic pathogen hash table of the reference genome of the respective oncogenic pathogen, where the corresponding hash table uses a seed length that is at least sixteen nucleotides in length to hash a plurality of reference seeds drawn from the reference genome of the respective oncogenic pathogen. In some embodiments, the hash table uses a seed length that is from 10 nucleotides to 30 nucleotides in length. In some embodiments, the hash table uses a seed length that is from 15 nucleotides to 25 nucleotides in length. In some embodiments, the seed length is between 18 nucleotides and 22 nucleotides. In some embodiments, the seed length is 20 nucleotides. In yet other embodiments, the hash table uses a seed length that is at least 7, 8, 9, 10, 11, 12, 13, 14, 15, 16, 17, 18, 19, 20, 21, 22, 23, 24, 25, 26, 27, 28, 29, 30, or more nucleotides in length. In some embodiments, the hash table uses a rolling window hash, in which the plurality of reference seeds overlap each other on each oncogenic pathogen reference construct.

Hash-based alignment algorithms require less computation time to identify possible alignments of a sequence read to a reference genome, because the algorithm does not search for each nucleotide individually. However, this can result in the identification of several putative matches for the sequence read in the reference construct. Accordingly, the system then determines which, if any, of the putative matches represents a true alignment with the sequence read. Accordingly, in some embodiments, the alignment (5098) of the sequence reads against the reference constructs for the oncogenic pathogens includes calculating a corresponding similarity score between the respective sequence read and putative matching loci in the reference genomes for the oncogenic pathogens. In some embodiments, the determination includes ranking the putative matches to the sequence read and determining whether the highest ranked alignment is significantly better enough than the other putative matches in order for a positive match to be assigned. In some embodiments, the one or more (putatively matched) locations (in the reference genome) include a plurality of locations that are ranked by their minimum edit distance thereby forming a ranked list of minimum edit distances, where the respective sequence read is determined to align to the human reference genome when a smallest minimum edit distance is smaller than a second most smallest minimum edit distance in the ranked list of minimum edit distances by a threshold amount. In other embodiments, the sequence read is putatively assigned to match to the locus in an oncogenic pathogen reference genome with the highest similarity score to the sequence read, e.g., regardless of whether that similarity score is significantly better than a similarity score for a second locus from an oncogenic pathogen reference construct. However, in some embodiments, a minimal threshold similarity must be met before any match is assigned.

The result of the alignment against the oncogenic pathogen reference constructs is the partitioning of the remaining sequencing reads into those sequence reads that map to an oncogenic pathogen reference construct and those sequence reads that do not map to an oncogenic pathogen reference construct (e.g., unaligned sequence reads 146).

### Competitive Alignment Between the Human Reference Genome and the Oncogenic Pathogen Reference Construct

As shown in FIG. 3, the result of the alignment of the unmapped sequence reads against the oncogenic pathogen reference constructs is the formation of a sub-plurality of sequence reads 315 that are putatively mapped to a locus in a reference construct for an oncogenic pathogen (e.g., aligned sequence reads 144). However, because high-throughput alignment methodologies, e.g., such as hash-based sequence alignment, are inexact, there is a significant rate of false positive and false negative alignments, both of which could artificially inflate the sequence count for a given oncogenic pathogen. In addition, the human reference genome used for the initial alignment does not contain all haplotypes and cannot account for genomic rearrangements, e.g., translocations, inversions, etc., that are not uncommon in cancer genomes. As such, human-derived sequence reads may have passed through the computational subtraction process and were subsequently matched to an oncogenic pathogen reference construct. Accordingly, in some embodiments, as shown in FIG. 3, these putative matches are confirmed by performing a competitive alignment of the sequence read against the human reference genome and the oncogenic pathogen reference construct, e.g., using sequence alignment module 130.

Accordingly, in some embodiments, the alignment (5098) of the sequence reads (e.g., aligned sequence reads 144) against reference constructs for the oncogenic pathogens includes (i) calculating a corresponding similarity score between the respective sequence read and the respective reference genome of the oncogenic pathogen in the plurality of oncogenic pathogens, (ii) labeling the respective sequence read as aligning with human reference genome when the best similarity score between the respective sequence read and the human reference genome exceeds the similarity score between the respective sequence read and the respective reference genome of the oncogenic pathogen in the plurality of oncogenic pathogens, and (iii) labeling the respective sequence read as aligning with a particular oncogenic pathogen in the plurality of oncogenic pathogens when the similarity score between the respective sequence read and the reference genome of the particular oncogenic pathogen exceeds the best similarity score between the respective sequence read and the human reference genome (5102), e.g., forming set 148 of aligned sequence reads.

In some embodiments, the similarity scores determined for the alignment between the sequence read and an oncogenic pathogen, as well as the similarity score determined for the alignment between the sequence read and the human reference genome, are the same similarity score determined when aligning the sequence read against the oncogenic pathogen reference construct and human reference genome, e.g., using a hash-based algorithm.

In some embodiments, the similarity scores determined for the alignment between the sequence read and an oncogenic pathogen, as well as the similarity score determined for the alignment between the sequence read and the human reference genome, are not the same similarity score determined when aligning the sequence read against the oncogenic pathogen reference construct and human reference genome, e.g., using a hash-based algorithm. Rather, in some embodiments, the sequence read is re-aligned to the human reference genome and the oncogenic pathogen reference construct using a local sequence alignment algorithm, which thereby generates a similarity score. A local sequence alignment algorithm compares subsequences of different lengths in the query sequence (e.g., sequence read) to subsequences in the subject sequence (e.g., reference construct) to create the best alignment for each portion of the query sequence. In contrast, global sequence alignment algorithms align the entirety of the sequences, e.g., end to end. Examples of local sequence alignment algorithms include the Smith-Waterman algorithm (see, for example, Smith and Waterman, J Mol. Biol., 147(1):195-97 (1981), which is incorporated herein by reference), Lalign (see, for example, Huang and Miller, Adv. Appl. Math, 12:337-57 (1991), which is incorporated by reference herein), and PatternHunter (see, for example, Ma B. et al., Bioinformatics, 18(3):440-45 (2002), which is incorporated by reference herein).

The result of the competitive alignment step described above is the formation of a sub-plurality of sequence reads 318 that have been positively mapped to an oncogenic pathogen reference construct.

### Normalization of Read Counts

In some embodiments, as shown in FIG. 3, read counts for the sequence reads 318 that are positively mapped to an oncogenic pathogen reference construct are normalized, e.g., to account for pull-down, amplification, and/or sequencing bias (e.g., mappability, GC bias etc.). See, for example, Schwartz et al., 2011, “Detection and Removal of Biases in the Analysis of Next-Generation Sequencing Reads,” PLoS ONE 6(1): e16685.doi:10.1371/ journal.pone.0016685; and Benjamini and Speed, 2012 “Summarizing and correcting the GC content bias in high-throughput sequencing,” Nucleic Acids Research 40(10) e72, each of which is hereby incorporated by reference.

### Strain Classification

In some embodiments, the hash-based alignment algorithm allows for alignment of a sequence read to an oncogenic pathogen at a family level, e.g., irrespective of which strain of the oncogenic pathogen the sequence originates. This is because hash-based algorithms, e.g., that use edit distance as a parameter, allow for intermediate non-alignment of the query and reference sequences in positive matches. However, in some cases, the identity of the particular strain of the oncogenic pathogen informs the optimal treatment regime for an afflicted subject. Accordingly, in some embodiments, as shown in FIG. 3, sequence reads 318 that have been positively mapped to an oncogenic pathogen (e.g., aligned sequence reads 146 in aligned sequence read set 147) are further classified as to the particular strain of the oncogenic pathogen, e.g., using oncogenic pathogen identification module 150.

In some embodiments, classification of the pathogen strain is performed by competitive alignment of the sequence read against a plurality of reference constructs for the various strains of the oncogenic pathogen. Generally, the competitive alignment is performed by aligning the sequence read to each reference construct, and determining a similarity score for the alignment. The similarity scores are then compared, and the sequence read is assigned to the strain corresponding to the highest similarity score. In some embodiments, the competitive alignment is performed using a local sequence alignment algorithm. As described above, local sequence alignment algorithms (such as the Smith-Waterman algorithm, Lalign, and PatternHunter), require more computational resources than hash-based mapping algorithms, but are more precise than hash-based mapping algorithms.

Accordingly, in some embodiments, the alignment (5098) of the sequence reads against reference constructs for the oncogenic pathogens is performed against a first database that includes at least one reference construct for HPV, at least one reference construct for EBV, and at least one reference construct for MCPyV, e.g., using an index-based alignment algorithm (such as a hash-based alignment algorithm). After one or more sequence reads are aligned to either the HPV reference construct, the EBV reference construct, or the MCPyV reference construct, a competitive alignment is performed between the sequence read and reference constructs for different strains of the HPV, EBV, or MCPyV, e.g., using a second database. In some embodiments, the first database includes at least reference constructs for HPV16, HPV18, and HPV33. In other embodiments, the first database only includes a reference construct for one of HPV16, HPV18, and HPV33. In some embodiments, the first database includes a consensus reference construct for two or more of HPV16, HPV18, and HPV33.

### Classification of Subject Infection

As shown in FIG. 3, counts of sequence reads 318 (e.g., aligned sequence reads 148) for each oncogenic pathogen, which may have been normalized, are then used to determine whether the subject is afflicted with the corresponding oncogenic pathogen. In some embodiments, this is done by tracking the total number of sequence reads mapped to each respective oncogenic pathogen reference construct, and determining (322) whether the total number meets a first threshold number of sequence reads, e.g., forming pathogen counts 156.

Accordingly, in some embodiments, method 5000 includes tracking (5104) for each respective oncogenic pathogen in the plurality of oncogenic pathogens, a number of sequence reads in the plurality of sequence reads that both (i) fail to align to the human reference genome and (ii) align to a reference genome of a respective oncogenic pathogen (e.g., sequence reads 318, as depicted in FIG. 3), thereby obtaining a sequence read count for each oncogenic pathogen in the plurality of oncogenic pathogens. For example, tallying a first number of sequence reads determined to map to an HPV16 reference construct, a second number of sequence reads that map to an EBV reference construct, and a third number of sequence reads that map to an MCPyV reference construct.

Then, method 5000 includes using (5106) the sequence read count for each oncogenic pathogen in the plurality of oncogenic pathogens to ascertain whether the subject is afflicted with an oncogenic pathogen (e.g., as illustrated in step 322 of FIG. 3). In some embodiments, the using identifies the subject as being afflicted with a respective oncogenic pathogen in the plurality of oncogenic pathogens when the read count for the respective oncogenic pathogen exceeds a threshold number of sequence reads in the plurality of sequence reads (5108). Generally, the threshold number of sequence reads is set such that numbers of sequence reads below the threshold correspond to noise in the data, rather than an actual infection in the subject. For instance, identification of just one or two sequences that map to a particular oncogenic pathogen does not correspond to actual infection in the subject. Accordingly, because the number of identified sequence reads would fall below the predetermined threshold, the system would classify the subject as not afflicted with that particular oncogenic pathogen.

Generally, a biological sample from a subject that is afflicted with an oncogenic pathogen results in the identification of from one hundred to several hundred sequence reads that map to the oncogenic pathogen reference construct, using the methods described herein. However, these methods can correctly identify infection at much lower numbers of corresponding sequence reads, e.g., at ten sequence reads or less. Accordingly, in some embodiments, threshold number of sequence reads is between seven and twenty-five sequence reads (5110). In one embodiment, the threshold number or sequence reads is ten sequence reads (5112). In some embodiments, the threshold number or sequence reads is 7, 8, 9, 10, 11, 12, 13, 14, 15, 16, 17, 18, 19, 20, 21, 22, 23, 24, 25, 26, 27, 28, 29, or 30 sequence reads.

In some embodiments, the method further identifies which strain of the oncogenic pathogen the subject has been afflicted with. For example, in some embodiments, method 5000 determines that the subject is afflicted with the oncogenic virus, and method 500 includes using the sequence reads that map to a reference genome of the oncogenic virus to determine a strain of the oncogenic virus from among a plurality of strains of the oncogenic virus. For instance, in some embodiments, the using determines that the subject is afflicted with the member of the papillomavirus family, and the method includes using the sequence reads that map to a reference genome of the member of the papillomavirus family to determine a strain of the member of the papillomavirus family from among a plurality of strains of the papillomavirus family (5116). In some embodiments, the strain of the member of the papillomavirus family is HPV16, HPV18, HPV31, HPV33, HPV35, HPV39, HPV45, HPV51, HPV52, HPV56, HPV58, HPV59 or HPV68 (5118).

Similarly, in some embodiments, the using determines that the subject is afflicted with the member of the herpes virus family, and the method includes using the sequence reads that map to a reference genome of the member of the herpes virus family to determine a strain of the member of the herpes virus family from among a plurality of strains of the herpes virus family (5120). In some embodiments, plurality of strains of the herpes virus family includes the Epstein-Barr virus (5122).

Similarly, in some embodiments, the using determines that the subject is afflicted with the member of the murine polyomavirus group, and the method includes using the sequence reads that map to a reference genome of the member of the murine polyomavirus group to determine a strain of the murine polyomavirus group from among a plurality of strains of the murine polyomavirus group (5124). In some embodiments, the strain in the plurality of strains of the murine polyomavirus group is Merkel cell polyomavirus (5126).

In some embodiments, no reference construct for the strain of the oncogenic pathogen the subject is afflicted with will exist. Accordingly, in some embodiments, de novo assembly of the sequence reads data is performed to identify the strain of the pathogen. Specifically, in some embodiments, the using determines that the subject is afflicted with a first oncogenic pathogen in the plurality of oncogenic pathogens, and the method also includes: subjecting the sequence reads for the first oncogenic pathogen in the plurality of sequence reads to de novo assembly thereby reconstructing a consensus sequence of a genome of the first oncogenic pathogen; comparing the genome of the first oncogenic pathogen to the respective reference genome of each strain in one or more known strains of the first oncogenic pathogen; and identifying the first oncogenic pathogen in the subject as a new strain of the first oncogenic pathogen when a homology between the genome of the first oncogenic pathogen and the reference genome of each strain in one or more known strains of the first oncogenic pathogen fails to satisfy a homology criterion (5128). Generally, the homology criteria is between about 80% and about 100%. In one embodiment, the homology criteria is 90% (5130). In other embodiments, the homology criteria is about 80, 81, 82, 83, 84, 85, 86, 87, 88, 89, 90%, 91%, 92%, 93%, 94%, or 95%.

### RNA Sequencing-Based Oncogenic Pathogen Detection

Another aspect of the present disclosure provides methods for discriminating between a first cancer condition and a second cancer condition in a subject, where the first cancer condition is associated with infection by a first oncogenic pathogen and the second cancer condition is associated with an oncogenic pathogen-free status. The method includes obtaining a dataset for the subject, the dataset including a plurality of abundance values, where each respective abundance value in the plurality of abundance values quantifies a level of expression of a corresponding gene, in a plurality of genes, in a cancerous tissue from the subject. The method then includes inputting the dataset to a classifier trained according to the any one of the methodologies described herein.

Another aspect of the present disclosure provides nucleic acid probes for discriminating between a first cancer condition and a second cancer condition in a human subject, where the first cancer condition is associated with an oncogenic pathogen infection and the second cancer condition is associated with an oncogenic pathogen-free status. The nucleic acid probes have nucleic acid sequences that are complementary or identical to sequences of the genes identified as differentially expressed in cancers associated with an oncogenic pathogen infection.

Another aspect of the present disclosure provides a method for discriminating between a first cancer condition and a second cancer condition in a subject with a first type of cancer, where the first cancer condition is associated with infection by a first oncogenic pathogen and the second cancer condition is associated with an oncogenic pathogen-free status. The method includes obtaining a dataset for the subject, the dataset having a plurality of abundance values (e.g., relative mRNA expression values), where each respective abundance value in the plurality of abundance values quantifies a level of expression of a corresponding gene, in a discriminating gene set, in a cancerous tissue from the subject. The method then includes inputting the dataset to a classifier trained to discriminate between at least the first cancer condition and the second cancer condition based on abundance values for the discriminating gene set in a cancerous tissue of a subject, thereby determining the cancer condition of the subj ect.

In some embodiments, the first type of cancer is breast cancer, lung cancer, prostate cancer, colorectal cancer, renal cancer, uterine cancer, pancreatic cancer, esophagus cancer, head/neck cancer, ovarian cancer, hepatobiliary cancer, cervical cancer, thyroid cancer, or bladder cancer.

In some embodiments, the dataset further includes a variant allele count for one or more variant alleles at one or more loci in the genome of the cancerous tissue from the subject.

In some embodiments, the first cancer condition is associated with infection by a first oncogenic pathogen selected from the group consisting of Epstein-Barr virus (EBV), hepatitis B virus (HBV), hepatitis C virus (HCV), human papilloma virus (HPV), human T-cell lymphotropic virus (HTLV-1), Kaposi’s associated sarcoma virus (KSHV), and Merkel cell polyomavirus (MCV).

In some embodiments, the first cancer condition is selected from the group consisting of cervical cancer associated with human papilloma virus (HPV), head and neck cancer associated with HPV, gastric cancer associated with Epstein-Barr virus (EBV), nasopharyngeal cancer associated with EBV, Burkitt lymphoma associated with EBV, Hodgkin lymphoma associated with EBV, liver cancer associated with hepatitis B virus (HBV), liver cancer associated with hepatitis C virus (HCV), Kaposi sarcoma associated with Kaposi’s associated sarcoma virus (KSHV), adult T-cell leukemia/lymphoma associated with human T-cell lymphotropic virus (HTLV-1), and Merkel cell carcinoma associated with Merkel cell polyomavirus (MCV).

In some embodiments, the first cancer condition is associated with infection by a human papillomavirus (HPV) oncogenic virus and the second cancer condition is associated with an HPV-free status, and the discriminating gene set includes at least five genes selected from the genes listed in Table 21. In some embodiments, the first cancer condition is cervical cancer associated with infection by a human papillomavirus (HPV). In some embodiments, the first cancer condition is head and neck cancer associated with infection by a human papillomavirus (HPV). In some embodiments, the discriminating gene set includes at least ten genes selected from the genes listed in Table 21. In some embodiments, the discriminating gene set includes at least twenty genes selected from the genes listed in Table 21. In some embodiments, the discriminating gene set includes at least all twenty-four of the genes listed in Table 21. In some embodiments, the dataset also includes a variant allele count for TP53 (ENSG00000141510) and CDKN2A (ENSG00000147889) in the genome of the cancerous tissue from the subject.

In some embodiments, the method also includes treating the subject for cervical cancer by, when the classifier result indicates that the human cancer patient is infected with an HPV oncogenic virus, administering a first therapy tailored for treatment of cervical cancer associated with an HPV infection, and when the classifier result indicates that the human cancer patient is not infected with an HPV oncogenic virus, administering a second therapy tailored for treatment of cervical cancer not associated with an HPV infection. In some embodiments, the first therapy tailored for treatment of cervical cancer associated with an HPV infection includes a therapeutic vaccine or an adoptive cell therapy. In some embodiments, the second therapy tailored for treatment of cervical cancer not associated with an HPV infection is chemotherapy. In some embodiments, the chemotherapy includes co-administration of cisplatin and a second therapeutic agent selected from the group consisting of 5-fluorouracil, paclitaxel, and bevacizumab.

In some embodiments, the method also includes treating the subject for head and neck cancer by, when the classifier result indicates that the human cancer patient is infected with an HPV oncogenic virus, administering a first therapy tailored for treatment of head and neck cancer associated with an HPV infection, and when the classifier result indicates that the human cancer patient is not infected with an HPV oncogenic virus, administering a second therapy tailored for treatment of head and neck cancer not associated with an HPV infection. In some embodiments, the first therapy tailored for treatment of head and neck cancer associated with an HPV infection includes a therapeutic vaccine, an immune checkpoint inhibitor, or a PI3K inhibitor. In some embodiments, the second therapy tailored for treatment of head and neck cancer not associated with an HPV infection includes chemotherapy. In some embodiments, the chemotherapy includes administration of cisplatin, and the second therapy also includes concurrent radiotherapy or postoperative chemoradiation.

In some embodiments, the first cancer condition is associated with infection by an Epstein-Barr virus (EBV) oncogenic virus and the second cancer condition is associated with an EBV-free status, and the discriminating gene set includes at least five genes selected from the genes listed in Table 4. In some embodiments, the first cancer condition is gastric cancer associated with infection by an Epstein-Barr virus (EBV). In some embodiments, the discriminating gene set includes all nine genes listed in Table 4. In some embodiments, the dataset also includes a variant allele count for TP53 (ENSG00000141510) and PIK3CA (ENSG00000121879) in the genome of the cancerous tissue from the subject.

In some embodiments, the method also includes treating the subject for gastric cancer by, when the classifier result indicates that the human cancer patient is infected with an EBV oncogenic virus, administering a first therapy tailored for treatment of gastric cancer associated with an EBV infection, and when the classifier result indicates that the human cancer patient is not infected with an EBV oncogenic virus, administering a second therapy tailored for treatment of gastric cancer not associated with an EBV infection. In some embodiments, the first therapy tailored for treatment of gastric cancer associated with an EBV infection includes an immune checkpoint inhibitor. In some embodiments, the second therapy tailored for treatment of gastric cancer not associated with an EBV infection includes chemotherapy. In some embodiments, the chemotherapy includes administration of a therapeutic agent selected from the group consisting of paclitaxel, carboplatin, cisplatin, 5-fluorouracil, and oxaliplatin.

In some embodiments, the method also includes treating the subject for cancer by, when the classifier result indicates that the human cancer patient is infected with the first oncogenic pathogen, administering a first therapy tailored for treatment of the first type of cancer associated with infection by the first oncogenic pathogen, and when the classifier result indicates that the human cancer patient is not infected with the first oncogenic pathogen, administering a second therapy tailored for treatment of the first type of cancer associated with an oncogenic pathogen-free status.

In some embodiments, the classifier was trained by a method including (1) obtaining a dataset comprising, for each respective subject in a plurality of subjects of a species: (i) a corresponding plurality of abundance values, wherein each respective abundance value in the corresponding plurality of abundance values quantifies a level of expression of a corresponding gene, in a plurality of genes, in a tumor sample of the respective subject, and (ii) an indication of cancer condition of the respective subject, wherein the indication of cancer condition identifies whether the respective subject has the first cancer condition or the second cancer condition, and wherein the plurality of subjects includes a first subset of subjects that are afflicted with the first cancer condition and a second subset of subjects that are afflicted with the second condition; (2) identifying the discriminating gene set using the corresponding plurality of abundance values and respective indication of the cancer condition of respective subjects in the plurality of subjects, wherein the discriminating gene set comprises a subset of the plurality of genes; and (3) using the respective abundance values for the discriminating gene set and the respective indication of cancer condition across the plurality of subjects to train a classifier to discriminate between the first cancer condition and the second cancer condition as a function of respective abundance values for the discriminating gene set.

### RNA Sequencing-Based Oncogenic Pathogen Detection

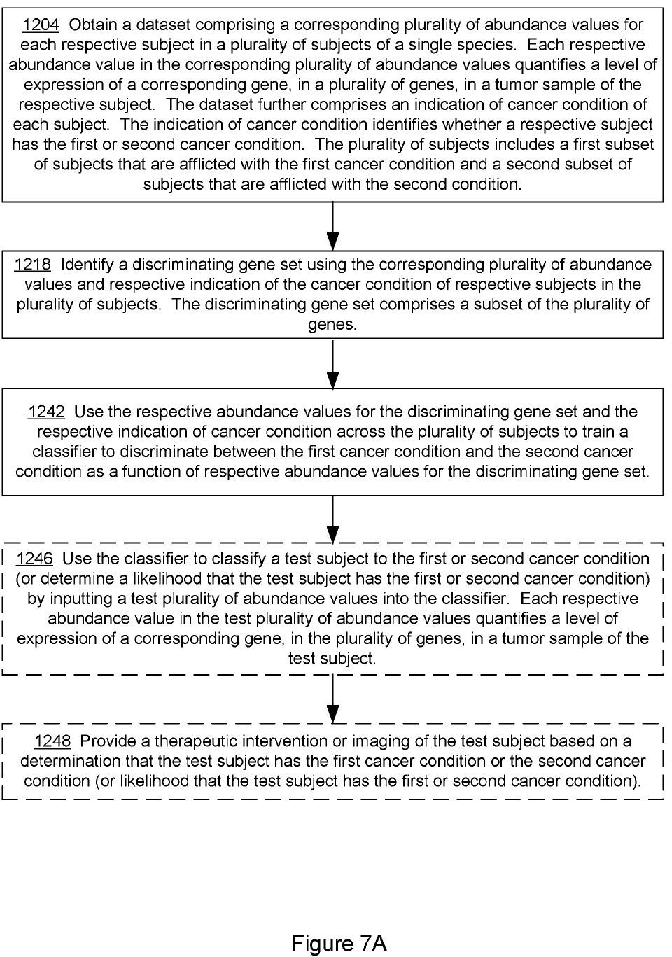

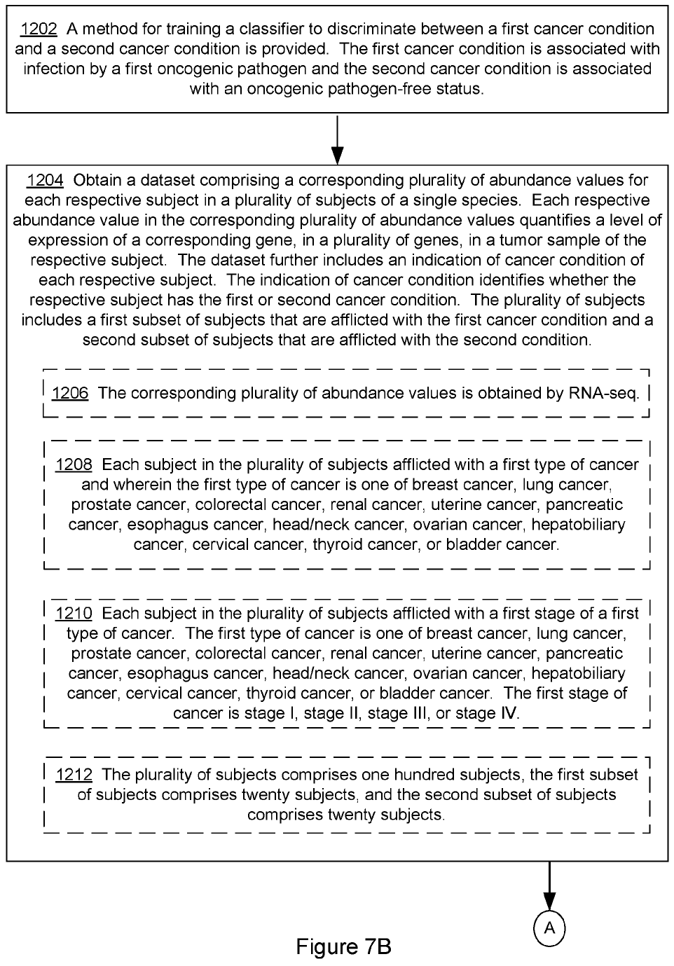

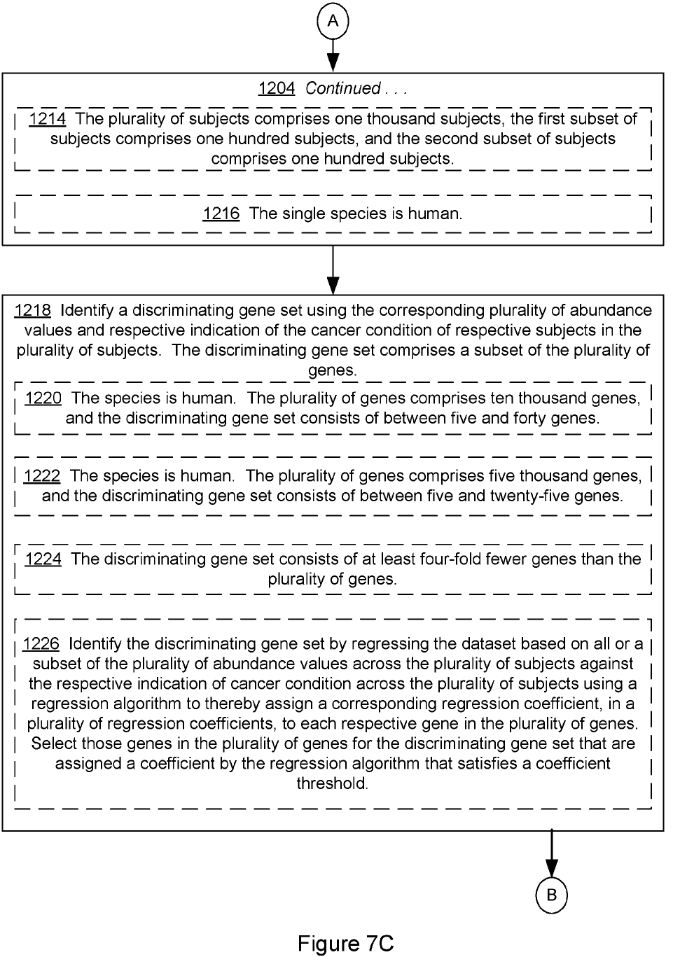

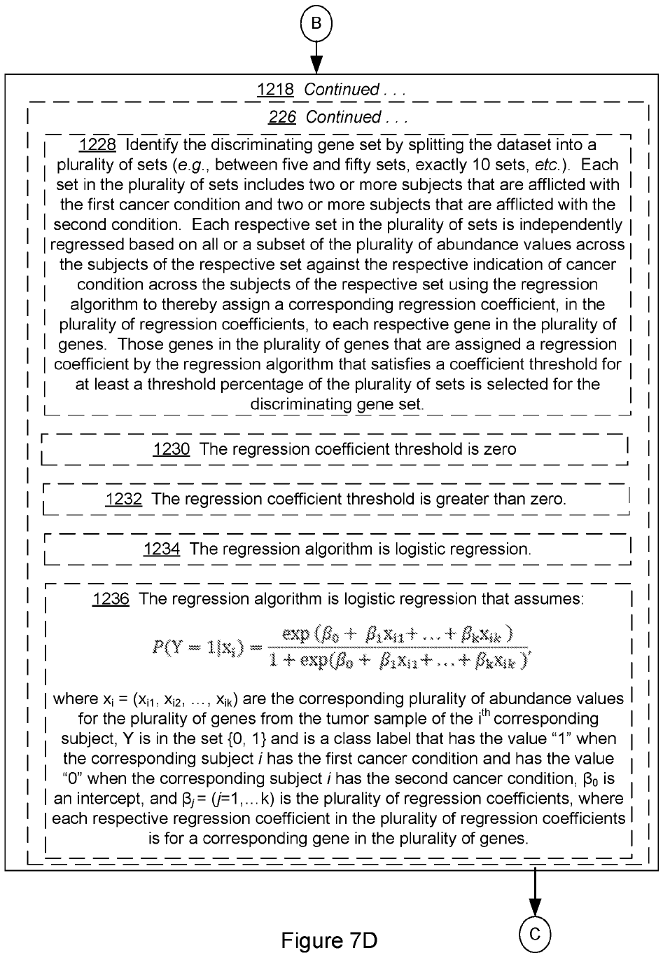

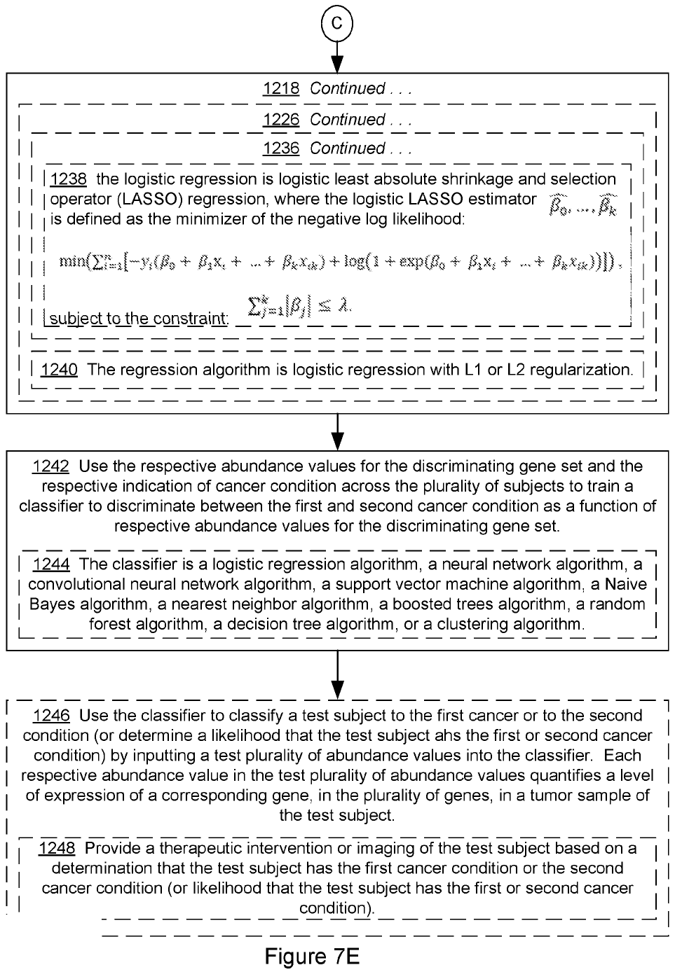

In some embodiments, the disclosure provides methods for discriminating between a first cancer condition and a second cancer condition in a human subject, where the first cancer condition is associated with infection by an oncogenic pathogen and the second cancer condition is associated with an oncogenic pathogen-free status. Generally, the methods include obtaining abundance data, e.g., relative expression levels, for a plurality of genes that are differentially expressed in cancerous tissue associated with one or more oncogenic pathogen infections and the same type of cancerous tissue that is not associated with an oncogenic pathogen infection. The abundance data is then input into a classifier that is trained to discriminate between the first cancer condition and the second cancer condition, at least in part, based on the abundance of the genes that are differentially expressed in the two types of cancerous tissues. Examples of the training of such classifiers are shown in FIG. 7, and further described in U.S. Pat. Application Publication No. 2020/0273576, which is incorporated herein by reference in its entirety, and specifically here for its description of classifier training in conjunction with the method shown in FIG. 2.

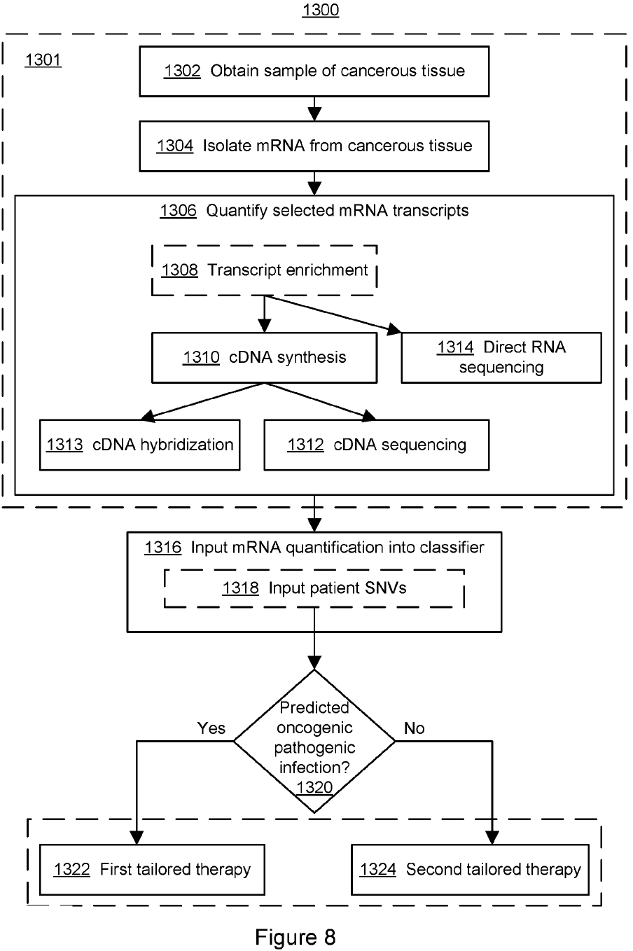

Many of the embodiments described below, in conjunction with FIG. 8, relate to analyses performed using expression data from the exome of a cancer patient, e.g., obtained from a sample of the cancerous tissue in the patient. Generally, these embodiments are independent and, thus, not reliant upon any particular expression data generation methods, e.g., sequencing, hybridization, and/or qPCR methodologies. However, in some embodiments, the methods described below include one or more steps (1301) of generating expression data.

In some embodiments, these methods include obtaining (1302) a sample of the cancerous tissue. Methods for obtaining samples of cancerous tissue are known in the art and are dependent upon the type of cancer being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient.

In some embodiments, mRNA is then isolated (1304) from the sample of the cancerous tissue. Many techniques for RNA isolation from a tissue sample are known in the art. For example, acid guanidinium thiocyanate-phenol-chloroform extraction (see, for example, Chomczynski and Sacchi, Nat Protoc, 1(2):581-85 (2006), the content of which is incorporated herein by reference, in its entirety, for all purposes), and silica bead/glass fiber adsorption (see, for example, Poeckh, T. et al., Anal Biochem., 373(2):253-62 (2008), the content of which is incorporated herein by reference, in its entirety, for all purposes). The selection of any particular RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed with the RNA sample.

In some embodiments, RNA is isolated from blood samples and/or tissue sections (e.g., a tumor biopsy) using commercially available reagents, for example, proteinase K, TURBO DNase-I, and/or RNA clean XP beads. In some embodiments, the isolated RNA is subjected to a quality control protocol to determine the concentration and/or quantity of the RNA molecules, including the use of a fluorescent dye and a fluorescence microplate reader, standard spectrofluorometer, or filter fluorometer.

In some embodiments, expression data is obtained directly from the isolated mRNA, e.g., by direct RNA sequencing (314). Methods for direct RNA sequencing are well known in the art. See, for example, Ozsolak F., et al., Nature 461:814-18 (2009), and Garalde, D.R., et al., Nat Methods, 15(3):201-206 (2018), the contents of which are incorporated herein by reference, in their entireties, for all purposes.

In other embodiments, expression data is obtained through a cDNA intermediate. Accordingly, in some embodiments, the isolated RNA is used to create a cDNA library via cDNA synthesis (310). In some embodiments, cDNA libraries are prepared from isolated RNA that is purified and selected for cDNA molecule size selection using commercially available reagents, for example Roche KAPA Hyper Beads. In another example, a New England Biolabs (NEB) kit may be used.

In some embodiments, cDNA library preparation includes ligation of adapters onto the cDNA molecules. For example, UDI adapters, such as Roche SeqCap dual end adapters, or UMI adapters (for example, full length or stubby Y adapters) may be ligated to the cDNA molecules. Adapters are nucleic acid molecules that may serve as barcodes to identify cDNA molecules according to the sample from which they were derived and/or to facilitate the downstream bioinformatics processing and/or the next generation sequencing reaction. The sequence of nucleotides in the adapters may be specific to a sample in order to distinguish samples. The adapters may facilitate the binding of the cDNA molecules to anchor oligonucleotide molecules on the sequencer flow cell and may serve as a seed for the sequencing process by providing a starting point for the sequencing reaction.

cDNA libraries may be amplified and purified using reagents, for example, Axygen MAG PCR clean up beads. Then the concentration and/or quantity of the cDNA molecules may be quantified using a fluorescent dye and a fluorescence microplate reader, standard spectrofluorometer, or filter fluorometer.

In some embodiments, both for direct RNA sequencing and prior to cDNA library construction, the isolated RNA is first enriched (1308) for a desired type of RNA (e.g., mRNA) or species (e.g., specific mRNA transcripts), prior to cDNA library construction. Methods of enriching for desired RNA molecules are also well known in the art. For example, mRNA molecules can be enriched, e.g., relative to other RNA molecules in a total RNA preparation, using oligo-dT affinity techniques (see, for example, Rio, D.C., et al., Cold Spring Harb Protoc., 2010 Jul 1;2010(7), the content of which is incorporated herein by reference, in its entirety, for all purposes). Specific mRNA transcripts can also be isolated, e.g., using hybridization probes that specifically bind to one or more mRNA sequences of interest.

In some embodiments, cDNA libraries are pooled and treated with reagents to reduce off-target capture, for example Human COT-1 and/or IDT xGen Universal Blockers, before being dried in a vacufuge. Pools may then be resuspended in a hybridization mix, for example, IDT xGen Lockdown, and probes may be added to each pool, for example, IDT xGen Exome Research Panel v1.0 probes, IDT xGen Exome Research Panel v2.0 probes, other IDT probe panels, Roche probe panels, or other probes. Pools may be incubated in an incubator, PCR machine, water bath, or other temperature-modulating device to allow probes to hybridize. Pools may then be mixed with Streptavidin-coated beads or another means for capturing hybridized cDNA-probe molecules, especially cDNA molecules representing exons of the human genome. In another embodiment, polyA capture may be used. Pools may be amplified and purified once more using commercially available reagents, for example, the KAPA HiFi Library Amplification kit and Axygen MAG PCR clean up beads, respectively.

cDNA library construction from isolated mRNAs is also well known in the art. In some embodiments, cDNA library construction is performed by first-strand DNA synthesis from the isolated mRNA using a reverse transcriptase, followed by second-strand synthesis using a DNA polymerase. Example methods for cDNA synthesis are described in McConnell and Watson, 1986, FEBS Lett. 195(1-2), pp. 199-202; Lin and Ying, 2003, Methods Mol Biol. 221, pp. 129-143, and Oh et al., 2003, Exp Mol Med. 35(6), pp. 586-90, the contents of which are hereby incorporated herein by reference, in their entireties, for all purposes.

The cDNA library may also be analyzed to determine the fragment size of cDNA molecules, which may be done through gel electrophoresis techniques and may include the use of a device such as a LabChip GX Touch. Pools may be cluster amplified using a kit (for example, Illumina Paired-end Cluster Kits with PhiX-spike in). In one example, the cDNA library preparation and/or whole exome capture steps may be performed with an automated system, using a liquid handling robot (for example, a SciClone NGSx).

The library amplification may be performed on a device, for example, an Illumina C-Bot2, and the resulting flow cell containing amplified target-captured cDNA libraries may be sequenced on a next generation sequencer, for example, an Illumina HiSeq 4000 or an Illumina NovaSeq 6000 to a unique on-target depth selected by the user, for example, 300x, 400x, 500x, 10,000x, etc. The next generation sequencer may generate a FASTQ, BCL, or other file for each patient sample or each flow cell.

If two or more patient samples are processed simultaneously on the same sequencer flow cell, reads from multiple patient samples may be contained in the same BCL file initially and then divided into a separate FASTQ file for each patient. A difference in the sequence of the adapters used for each patient sample could serve the purpose of a barcode to facilitate associating each read with the correct patient sample and placing it in the correct FASTQ file.

Methods for mRNA sequencing are well known in the art. In some embodiments, the mRNA sequencing is performed by whole exome sequencing (WES). Generally, WES is performed by isolating RNA from a tissue sample, optionally selecting for desired sequences and/or depleting unwanted RNA molecules, generating a cDNA library, and then sequencing the cDNA library (1312), for example, using next generation sequencing (NGS) techniques. For a review of the use of whole exome sequencing techniques in cancer diagnosis, see, Serratì et al., 2016, Onco Targets Ther. 9, pp. 7355-7365, the content of which is hereby incorporated herein by reference, in its entirety, for all purposes.

Next generation sequencing methods are also well known in the art, including synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators.

In some embodiments, the sequence reads may be aligned to a reference exome or reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. Non-limited examples of well-known software for assembling and managing transcriptome information from RNA-seq data include TopHat and Cufflinks, see, Trapnell et al., 2012, Nat Protoc. 7(3), pp. 562-578, the content of which is hereby incorporated herein by reference, in its entirety, for all purposes. See, also, Hintzsche et al., 2016, Int J Genomics 7983236, the content of which is hereby incorporated herein by reference, in its entirety, for all purposes.

In other embodiments, expression data is generated by hybridization (1313) of the cDNA library, e.g., using a microarray. The use of microarray-based gene profiling to identify differential gene expression following pathogen infection is known in the art. For example, see, Adomas et al., 2008, Tree Physiol. 28(6), pp. 885-897, the content of which is hereby incorporated herein by reference, in its entirety, for all purposes. Similarly, in other embodiments, yet other methods for quantifying expression based on a cDNA library are used, for example, quantitative real-time PCR (RT-qPCR). See, for example, Wagner, 2013, Methods Mol Biol. 1027, pp. 19-45, the content of which is hereby incorporated herein by reference, in its entirety, for all purposes.

As illustrated with respect to FIG. 8, in some embodiments, method 1300 is performed, at least partially, at a computer system (e.g., computer system 1100 in FIG. 6) having one or more processors, and memory storing one or more programs for execution by the one or more processors for discriminating between a first cancer condition and a second cancer condition in a subject, where the first cancer condition is associated with infection by a first oncogenic pathogen and the second cancer condition is associated with an oncogenic pathogen-free status. Some operations in method 1300 are, optionally, combined and/or the order of some operations is, optionally, changed.

In some embodiments, the method includes obtaining a dataset for the subject, the dataset including a plurality of abundance values, where each respective abundance value in the plurality of abundance values quantifies a level of expression of a corresponding gene, in a plurality of genes, in a cancerous tissue from the subject. In some embodiments, the obtained abundance values are determined according to any of the methodologies described with respect to sub-method 1301. In some embodiments, the abundance data is pre-generated and communicated to computer system 1100 over a network, e.g., using network interface 1104. Method 1300 then includes inputting (1316) the dataset to a classifier trained for discriminating between a first cancer condition and a second cancer condition in a human subject, where the first cancer condition is associated with infection by an oncogenic pathogen and the second cancer condition is associated with an oncogenic pathogen-free status. Examples of such classifiers are provided above in conjunction with FIG. 7, and in conjunction with the description of FIG. 2 in U.S. Pat. Application Publication No. 2020/0273576, which is incorporated herein by reference in its entirety. Thereby, the method determines (1320) whether the subject has the first cancer condition, associated with the oncogenic pathogen infection, or the second cancer condition, that is not associated with the oncogenic pathogen infection.

In some embodiments, method 1300 also includes inputting a variant allele count for one or more variant alleles at one or more loci in the genome of the cancerous tissue from the subject into the classifier. That is, in some embodiments, the classifier is also trained against data relating to the presence or absence of one or more variant alleles in subjects with cancers that are either associated with an oncogenic pathogen infection or not associated with an oncogenic pathogen infection. In some embodiments, the one or more variant alleles are selected from variant alleles in a gene selected from the group consisting of TP53 (ENSG00000141510), CDKN2A (ENSG00000147889), and PIK3CA (ENSG00000121879).

In some embodiments, the subject is afflicted with breast cancer, lung cancer, prostate cancer, colorectal cancer, renal cancer, uterine cancer, pancreatic cancer, esophagus cancer, head/neck cancer, ovarian cancer, hepatobiliary cancer, cervical cancer, thyroid cancer, or bladder cancer.

In some embodiments, the first cancer condition is associated with infection by a first oncogenic pathogen selected from Epstein-Barr virus (EBV), hepatitis B virus (HBV), hepatitis C virus (HCV), human papilloma virus (HPV), human T-cell lymphotropic virus (HTLV-1), Kaposi’s associated sarcoma virus (KSHV), and Merkel cell polyomavirus (MCV).

More specifically, in some embodiments, the first cancer condition is selected from cervical cancer associated with human papilloma virus (HPV), head and neck cancer associated with HPV, gastric cancer associated with Epstein-Barr virus (EBV), nasopharyngeal cancer associated with EBV, Burkitt lymphoma associated with EBV, Hodgkin lymphoma associated with EBV, liver cancer associated with hepatitis B virus (HBV), liver cancer associated with hepatitis C virus (HCV), Kaposi sarcoma associated with Kaposi’s associated sarcoma virus (KSHV), adult T-cell leukemia/lymphoma associated with human T-cell lymphotropic virus (HTLV-1), and Merkel cell carcinoma associated with Merkel cell polyomavirus (MCV). For a summary of cancer conditions known to be associated with an oncogenic viral infection, see, de Flora, 2011, “The prevention of infection-associated cancers,” Carcinogenesis 32, pp. 787-795.

Accordingly, when the first cancer condition is a particular type of cancer associated with a particular oncogenic pathogen, the second cancer condition is the same particular type of cancer associated with no infection of the particular oncolytic pathogen. For example, when the first cancer condition is cervical cancer associated with a human papilloma virus (HPV) infection, the second cancer condition is cervical cancer that is not associated with a human papilloma virus (HPV) infection. Further, as described above, the classifier used to discriminate between the two cancer conditions is trained against a dataset including at least gene abundance values (e.g., mRNA expression profiles) from subjects known to have cervical cancer associated with a human papilloma virus (HPV) infection and from subjects known to have cervical cancer that is not associate with a human papilloma virus (HPV) infection.

In some embodiments, the method further includes treating the subject with either a first therapy (1322) tailored for treatment of the first cancer condition, associated with the oncogenic pathogenic infection, or a second therapy (1324) tailored for treatment of the second cancer condition, not associated with the oncogenic pathogen infection.

Accordingly, in one embodiment, a method is provided for treating a cancer in a human cancer patient. The method includes determining whether the patient is infected with an oncogenic pathogen linked to the pathology of the cancer by obtaining a dataset for the patient, the dataset including a plurality of abundance values, and inputting the dataset into a classifier trained to discriminate between at least a first cancer condition associated with an infection of the oncogenic pathogen and a second cancer condition that is not associated with an infection of the oncogenic pathogen. Each abundance value in the dataset quantifies a level of expression of a corresponding gene found to be differentially expressed in cancers associated with an infection of the oncogenic pathogen and cancers that are not associated with an infection of the oncogenic pathogen. In some embodiments, the genes for which abundance values are used to discriminate between cancer conditions for any particular type of cancer are selected according to any of the selection methodologies described above with reference to FIG. 7 and in conjunction with the description of FIG. 2 in U.S. Pat. Application Publication No. 2020/0273576, which is incorporated herein by reference in its entirety. Similarly, in some embodiments, the classifier used is trained according to any of the training methodologies described above with reference to FIG. 7, and in conjunction with the description of FIG. 2 in U.S. Pat. Application Publication No. 2020/0273576, which is incorporated herein by reference in its entirety.

In some embodiments, when the subject is determined to have a first cancer condition, associated with an oncogenic pathogen infection, the method includes assigning and/or administering immunotherapy to the subject. In some embodiments, when the subject is determined to have a second cancer condition, that is not associated with an oncogenic pathogen infection, the method includes assigning and/or administering chemotherapy to the subject.

As summarized in Table 20, several clinical trials are ongoing for the treatment of virally associated tumors. Accordingly, in some embodiments, the methods described herein include assigning and/or administering a treatment for a particular cancer associated with a particular oncogenic viral infection, as listed in Table 20. For example, in some embodiments, upon a determination that the subject has a phase 3 cervical cancer associated with an HPV infection, the subject is assigned and/or administered a therapeutically effective dosing regimen of axalimogene filolisbac, which is a live attenuated Listeria monocytogenes transfected with plasmids encoding the HPV-16E7 protein fused to a truncated fragment of the Lm protein listeriolysin O.

TABLE 20

Clinical trials for the treatment of cancers associated with oncogenic viral infections

| | | | |
|---|---|---|---|
| Therapy | Mechanism of Action | Virus | Cancer / Stage of Development / Clinical Trial |
| Axalimogene filolisbac (AXAL/ADXS 11-001) | Therapeutic vaccine | HPV | Phase 3 cervical cancer (AIM2CERV; NCT02853604); Phase 2 NSCLC (NCT02531854); Phase ½ HNSCC (NCT02291055) |
| TG4001 | Therapeutic vaccine | HPV | Phase ½ HNSCC (NCT03260023) |
| GX-188E | Therapeutic vaccine | HPV | Phase ½ cervical cancer (NCT03444376) |
| VGX-3100 | Therapeutic vaccine | HPV | Phase 3 cervical cancer (REVEAL; NCT03185013); Phase 2 vulval cancer (NCT0318-684) |
| MEDI-0457 | Therapeutic vaccine | HPV | Phase 2 HPV+ cancer (NCT03439085); Phase ½ HNSCC (NCT03162224) |
| INO-3106 | Therapeutic vaccine | HPV | Phase 1 HPV+ cancers (NCT02241369) |
| TA-CIN | Therapeutic vaccine | HPV | Phase 1 cervical cancer (NCT02405221) |
| TA-HPV | Therapeutic vaccine | HPV | Phase 1 cervical cancer (NCT00788164) |
| ISA-101 | Therapeutic vaccine | HPV | Phase 2 HNSCC (NCT03258008) |
| PepCan | Therapeutic vaccine | HPV | Phase 2 cervical cancer (NCT02481414) |
| Nivolumab (Opdivo) | Immune checkpoint inhibitor | HPV | Phase 2 HNSCC (NCT03342911) |
| AMG319 | PI3K inhibitor | HPV | Phase 2 HNSCC (NCT02540928) |
| BKM120 | PI3K inhibitor | HPV | Phase 1 HNSCC (NCT02113878) |
| HPV-specific T cells | Adoptive cell therapy | HPV | Phase 1 HPV+ tumors (NCT02379520); Phase 1 vulvar cancers (NCT03197025) |
| ATA 129 | Adoptive cell therapy | EBV | Phase 3 EBV+ lymphoproliferative disease (NCT03394365/ALLELE, NCT03392142/MATCH) |
| EBVST | Adoptive cell therapy | EBV | Phase 3 EBV+ nasopharyngeal carcinoma (NCT02578641) |
| CMD-003 | Adoptive cell therapy | EBV | Phase 2 EBV+ lymphomas (NCT02763254, NCT01948180/CITADEL) |
| Ibrutinib | BTK inhibitor | EBV | Phase 2 EBV+ DLBCL (NCT02670616) |
| Pembrozilumab | Immune checkpoint inhibitor | EBV | Phase 2 EBV+ gastric cancer (NCT03257163); Phase 1 KSHV+ Kaposo sarcoma (NCT02595866) |
| Nivolumab | Immune checkpoint inhibitor | EBV | Phase 2 EBV+ lymphoproliferative disorders and NHL (NCT03258567) |
| Avelumab | Immune checkpoint inhibitor | MCV | Phase ½ MCV+ MCC (NCT02584829) |
| Talimogene laherparepvec | Vaccine | MCV | Phase 2 MCV+ MCC (NCT02819843) |
| Sapanisertib | mTOR inhibitor | MCV | Phase ½ MCV+ MCC (NCT02514824) |

### HPV Oncogenic Viral Infections

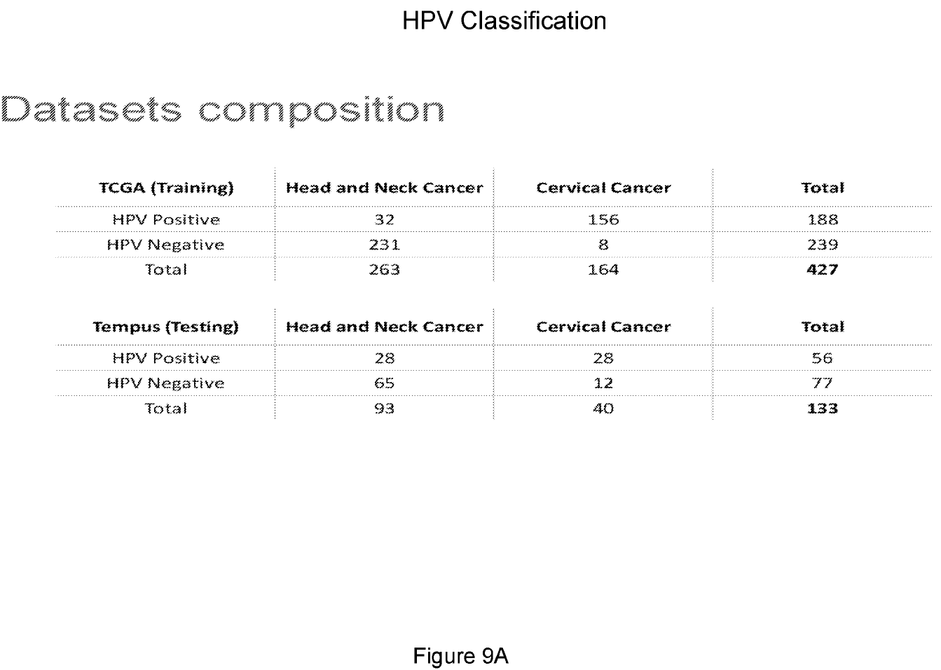

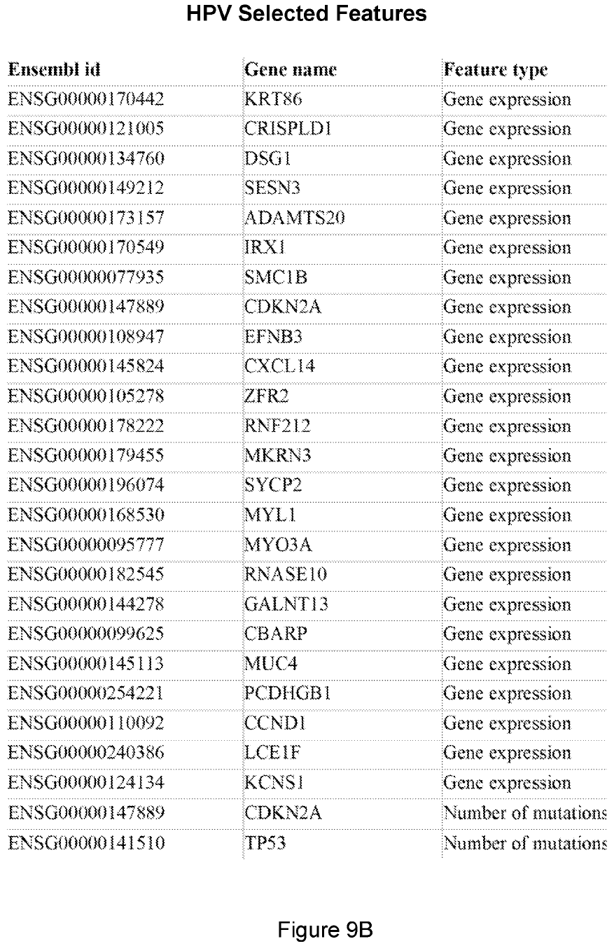

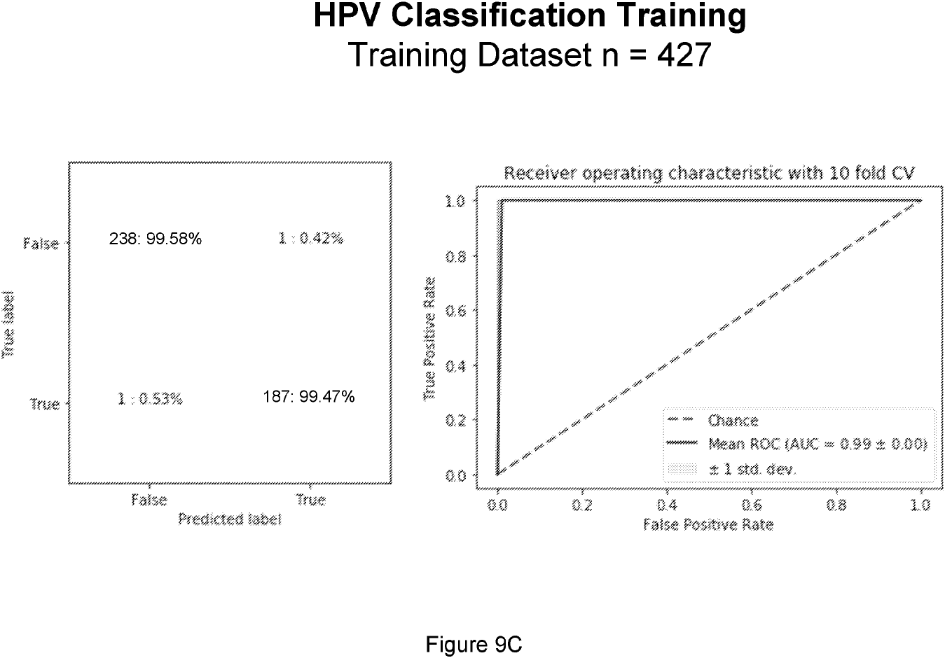

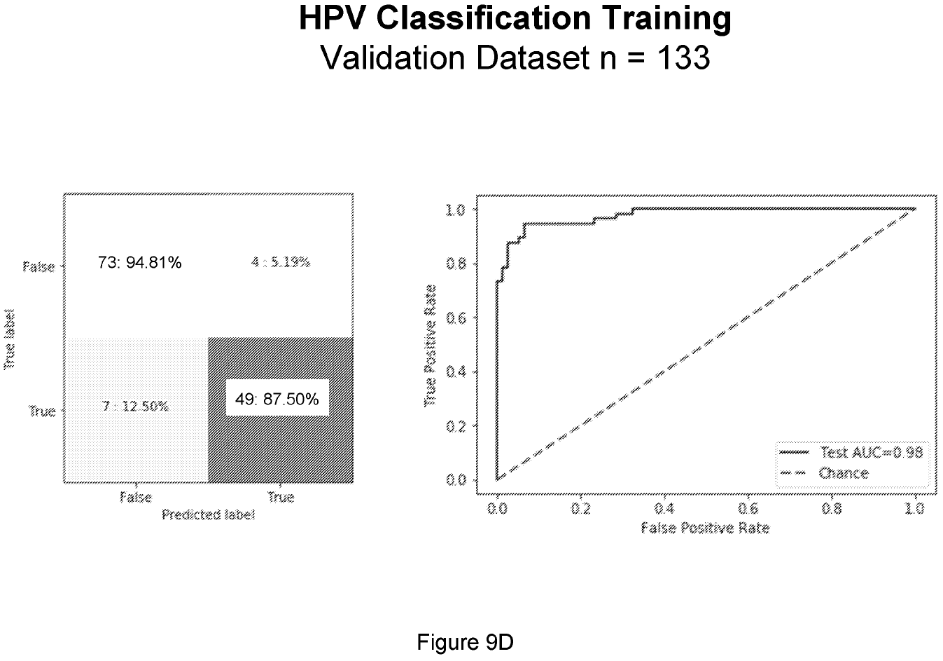

In some embodiments, the methods described herein relate to classification and/or treatment of cancers known to be associated with a human papillomavirus (HPV) infection. As reported in Example 8 below, the twenty-four genes listed in Table 21, and shown in FIG. 9B, were found to be differentially expressed in at least eight of the ten training sets formed from expression data of cervical or head and neck cancers with known HPV statuses in The Cancer Genome Atlas (TCGA). Accordingly, in some embodiments the expression levels of one or more of the genes listed in Table 21 are used for the classification of a cervical cancer or a head and neck cancer as either associated with an HPV infection or not associated with an HPV infection. In some embodiments, expression levels of at least 2, 3, 4, 5, 6, 7, 8, 9, 10, 11, 12, 13, 14, 15, 16, 17, 18, 19, 20, 21, 22, 23, or all 24 of the genes listed in Table 21 are used for the classification of a cervical cancer or a head and neck cancer as either associated with an HPV infection or not associated with an HPV infection.

TABLE 21

Genes found to be differentially expressed in at least 80% of the cervical cancer or head and neck cancer training sets derived from the TCGA database

| | |
|---|---|
| ENSEMBL ACCESSION ID | GENE NAME |
| ENSG00000170442 | KRT86 |
| ENSG00000121005 | CRISPLD 1 |
| ENSG00000134760 | DSG1 |
| ENSG00000149212 | SESN3 |
| ENSG00000173157 | ADAMTS20 |
| ENSG00000170549 | IRX1 |
| ENSG00000077935 | SMC1B |
| ENSG00000147889 | CDKN2A |
| ENSG00000108947 | EFNB3 |
| ENSG00000145824 | CXCL14 |
| ENSG00000105278 | ZFR2 |
| ENSG00000178222 | RNF212 |
| ENSG00000179455 | MKRN3 |
| ENSG00000196074 | SYCP2 |
| ENSG00000168530 | MYL1 |
| ENSG00000095777 | MYO3A |
| ENSG00000182545 | RNASE10 |
| ENSG00000144278 | GALNT13 |
| ENSG00000099625 | C19orf26 |
| ENSG00000145113 | MUC4 |
| ENSG00000254221 | PCDHGB1 |
| ENSG00000110092 | CCND1 |
| ENSG00000240386 | LCE1F |
| ENSG00000124134 | KCNS1 |

In one embodiment, a method is provided for discriminating between a first cancer condition and a second cancer condition in a human subject, wherein the first cancer condition is associated with infection by a human papillomavirus (HPV) oncogenic virus and the second cancer condition is associated with an HPV-free status. The method includes obtaining a dataset for the subject, e.g., as described above with reference to FIG. 8. The dataset includes a plurality of abundance values from the subject, where each respective abundance value in the plurality of abundance values quantifies a level of expression of a corresponding gene, in a plurality of genes, in a cancerous tissue from the subject. In some embodiments, the plurality of genes includes at least five genes selected from the genes listed in Table 21. The method then includes inputting the dataset to a classifier trained to discriminate between at least the first cancer condition and the second cancer condition based on the abundance values of the plurality of genes. In some embodiments, the classifier is trained in accordance with any of the methodologies described above, with respect to FIG. 7, and in conjunction with the description of FIG. 2 in U.S. Pat. Application Publication No. 2020/0273576, which is incorporated herein by reference in its entirety.

In some embodiments, the first cancer condition is cervical cancer associated with an HPV infection, and the second cancer condition is cervical cancer that is not associated with an HPV infection. In some embodiments, the first cancer condition is head and neck cancer associated with an HPV infection, and the second cancer condition is head and neck cancer that is not associated with an HPV infection. In some embodiments, the head and neck cancer is a specific form of head and neck cancer, e.g., hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer, or salivary gland cancer.

In some embodiments, the plurality of genes includes at least ten of the genes listed in Table 21. In some embodiments, the plurality of genes includes at least fifteen of the genes listed in Table 21. In some embodiments, the plurality of genes includes at least twenty of the genes listed in Table 21. In some embodiments, the plurality of genes includes all of the genes listed in Table 21. In some embodiment, the plurality of genes includes one or more genes that are not listed in Table 21, e.g., 1, 2, 3, 4, 5, 6, 7, 8, 9, 10, or more of the genes not listed in Table 21. In some embodiments, the plurality of genes includes no more than 20 genes. In some embodiments, the plurality of genes includes no more than 25 genes. In some embodiments, the plurality of genes includes no more than 50 genes. In some embodiments, the plurality of genes includes no more than 10, 15, 20, 25, 30, 35, 40, 50, 60, 70, 80, 90, 100, 125, 150, 175, 200, 250, or 300 genes.

In some embodiments, the dataset also includes a variant allele count for one or more alleles at one or more loci in the genome of the cancerous tissue from the subject. In some embodiments, the variant allele count is either 1, representing a state in which the subject carries the variant allele, or 0, representing a state in which the subject does not carry the variant allele. In some embodiments, the variant allele is a somatic variant, originating from the germ line of the subject. In some embodiments, the variant allele is a cancer-derived variant, originating from the cancerous tissue. In some embodiments, the variant allele is located in the TP53 (ENSG00000141510) or CDKN2A (ENSG00000147889) gene.

In some embodiments, the classifier is trained for determining the HPV status of a test subject having an HPV-associated cancer selected from cervical cancer, head and neck squamous cell carcinoma, ovarian cancer, penile cancer, pharyngeal cancer, anal cancer, vaginal cancer, and vulvar cancer. In some embodiments, the classifier is trained for determining the HPV status of a test patient having a specific HPV-associated cancer, e.g., cervical cancer, head and neck squamous cell carcinoma, ovarian cancer, penile cancer, pharyngeal cancer, anal cancer, vaginal cancer, or vulvar cancer. However, as classifier training is generally improved by increasing the size of the training dataset, in some embodiments, the classifier is trained against data from patients that have two or more types of HPV-associated cancers, e.g., two, three, four, five, six, seven, or all eight of cervical cancer, head and neck squamous cell carcinoma, ovarian cancer, penile cancer, pharyngeal cancer, anal cancer, vaginal cancer, and vulvar cancer. In a particular embodiment, exemplified by Example 8, the classifier is trained against subjects having either head and neck squamous cell carcinoma or cervical cancer. However, in some embodiments, a classifier trained against patients having one or more types of HPV-associated cancer is useful for determining the HPV status of a patient having a different type of HPV-associated cancer.

In some embodiments, the features of the classifier include abundance values for a plurality of genes selected from those listed in Table 21, e.g., KRT86, CRISPLD1, DSG1, SESN3, DAMTS20, IRX1, SMC1B, CDKN2A, EFNB3, CXCL14, ZFR2, RNF212, MKRN3, SYCP2, MYL1, MYO3A, RNASE10, GALNT13, C19orf26, MUC4, PCDHGB1, CCND1, LCE1F, and KCNS1. As reported below, e.g., in reference to Example 8, these twenty-four genes were found to be differentially expressed, dependent upon the HPV status of the subject, in at least eight of the ten training sets formed from expression data of cervical or head and neck cancers with known HPV statuses in The Cancer Genome Atlas (TCGA). However, the skilled artisan will appreciate that, in some instances, the use of different training data sets may yield different results, e.g., one or more of these genes may not be informative in at least 80% of training folds and/or one or more genes found not to be informative in at least 80% of training folds in the study reported in Example 21 may be informative. These differences may arise, for example, when different criteria are used to select the training population, e.g., different inclusion and/or exclusion criteria such as cancer type, personal characteristics (e.g., age, gender, ethnicity, family history, smoking status, etc.), or simply by using a smaller or larger data set.

Accordingly, in some embodiments, the features of the classifier include at least five of the genes listed in Table 21. In some embodiments, the features of the classifier include at least ten of the genes listed in Table 21. In some embodiments, the features of the classifier include at least fifteen of the genes listed in Table 21. In some embodiments, the features of the classifier include at least twenty of the genes listed in Table 21. In some embodiments, the features of the classifier include all twenty-four of the genes listed in Table 21. In some embodiments, the features of the classifier include 5, 6, 7, 8, 9, 10, 11, 12, 13, 14, 15, 16, 17, 18, 19, 20, 21, 22, 23, or all 24 of the genes listed in Table 21. Further, in some embodiments, the features of the classifier include the abundance values for one or more genes not listed in Table 21. In some embodiments, the features of the classifier include abundance values for 1, 2, 3, 4, 5, 6, 7, 8, 9, 10 or more genes not listed in Table 21. In some embodiments, the features of the classifier include the abundance values for 1-10 genes not listed in Table 21. In some embodiments, the features of the classifier include the abundance values for 1-5 genes not listed in Table 21. In other embodiments, the features of the classifier do not include the abundance values for any genes not listed in Table 21.

Further, the skilled artisan will also appreciate that some features, e.g., abundance values for a particular gene, will be more informative than other features in a particular classifier. One measure of the predictive power of respective features in a classifier based on multiple features is the regression coefficient calculated for the features during training of the model. Regression coefficients describe the relationship between each feature and the response of the model. The coefficient value represents the mean change in the response given a one-unit increase in the feature value. As such, at least for variables of the same type, the magnitude, e.g., absolute value, of a regression coefficient is correlated with the importance of the feature in the model. That is, the higher the magnitude of the regression coefficient, the more important the variable is to the model. For instance, as reported in Example 7, in a particular support vector machine (SVM) classifier trained against the abundance values of all twenty-four of the genes listed in Table 21, as well as a variant allele status for the TP53 and CDKN2A genes, only six of the 24 genes had regression coefficients with magnitudes of at least 0.5-CDKN2A (1.13), SMC1B (1.02), EFNB3 (-0.97), KCNS1 (0.74), CCND1 (-0.65), and RNF212 (0.517).

As such, the skilled artisan may select a feature set that includes less than all of the genes listed in Table 21 based, at least in part, upon the importance of the respective features in one or more classification models. For instance, in some embodiments, one or more genes with lower predictive power in a classification model may be left out during classifier training. For example, in some embodiments, the features of the classifier include at least the gene expression features listed in Table 23 with a regression coefficient of at least 0.5, e.g., CDKN2A, SMC1B, EFNB3, KCNS1, CCND1, and RNF212. In some embodiments, the features of the classifier include at least the gene expression features listed in Table 23 with a regression coefficient of at least 0.4. In some embodiments, the features of the classifier include at least the gene expression features listed in Table 23 with a regression coefficient of at least 0.3. In some embodiments, the features of the classifier include at least the gene expression features listed in Table 23 with a regression coefficient of at least 0.2. In some embodiments, the features of the classifier include at least the gene expression features listed in Table 23 with a regression coefficient of at least 0.1.

Similarly, the size of the feature set may be affected by which features are included and/or excluded. For instance, in some embodiments, if particular features having high predictive power are included in a classification model, fewer total features may be included in the model. For instance, in some embodiments, if the abundance values for SMC1B, CDKN2A, and EFNB3 are included in the model, the abundance values for no more than two of the other genes whose abundance values are used as features in Table 23 need to be included in the model. Accordingly, in some embodiments, the features of the classifier include abundance values for SMC1B, CDKN2A, and EFNB3, and at least two other genes whose abundance values are used as features in Table 23. In some embodiments, the features of the classifier include abundance values for SMC1B, CDKN2A, and EFNB3, and at least five other genes whose abundance values are used as features in Table 23. In some embodiments, the features of the classifier include abundance values for SMC1B, CDKN2A, and EFNB3, and at least ten other genes whose abundance values are used as features in Table 23. In some embodiments, the features of the classifier include abundance values for SMC1B, CDKN2A, and EFNB3, and at least fifteen other genes whose abundance values are used as features in Table 23.

Similarly, in some embodiments, if features having high predictive power are excluded from the classification model, more of the other features may be included in the model. For instance, in some embodiments, if the abundance values for one or more of SMC1B, CDKN2A, and EFNB3 are not included in the model, the abundance values for at least fifteen of the other whose abundance values are used as features in Table 23 are included in the model. In some embodiments, if the abundance values for one or more of SMC1B, CDKN2A, and EFNB3 are not included in the model, the abundance values for at least twenty of the other genes whose abundance values are used as features in Table 23 are included in the model. In some embodiments, if the abundance values for one or more of SMC1B, CDKN2A, and EFNB3 are not included in the model, the abundance values for at least 15, 16, 17, 18, 19, 20, or all 21 of the other genes whose abundance values are used as features in Table 23 are included in the model.

Of course, other metrics are also available for evaluating the importance of a feature in a model, such as standardized regression coefficients and change in R-squared when the comparing the output of a model having the feature to the output of a model that is identical except that it lacks the feature.

When selecting a feature set, the skilled artisan will also consider the degree to which features are correlated to each other. Correlation is a statistical measure of how linearly dependent two variables are upon each other. As such, two correlated features provide duplicative information to a predictive model, which can be detrimental to a classifier. As such, there are several reasons why a correlated feature may be excluded from a model. For instance, removing a correlated feature will make the algorithm faster, as the larger the number of features in a classifier the more computations that need to be made. Removing a correlated feature may also remove harmful bias, arising from the correlation, from a model. Finally, removing a correlated feature may make the model more interpretable.

As such, the skilled artisan may select a feature set that includes less than all of the genes listed in Table 21 based, at least in part, upon the correlation between respective features in one or more classification models. In some embodiments, the selection to remove one or the other feature of a correlated feature set is informed by predictive powers of the two features, e.g., their respective regression coefficients. For example, the gene expression values for ENSG00000105278 (CXCL14) and ENSG00000077935 (SMC1B) are highly correlated in the feature set listed in Table 21 (correlation = 0.718983175). Accordingly, in some embodiments, the feature set does not include either CXCL14 or SMC1B. In some embodiments, CXCL14, rather than SMC1B is excluded from the feature set because, as reported in Table 23, SMC1B has a higher regression coefficient (1.02) than CXCL14 (-0.29) in the SVM model described in Example 3.

As reported in Table 24, ten pairs of gene expression features have a correlation of at least 0.6. Accordingly, in some embodiments, a feature in at least one pair of features having a correlation of at least 0.6 is excluded from the model. In some embodiments, a feature in at least two pairs of features having a correlation of at least 0.6 is excluded from the model. In other embodiments, a feature in at least 3, 4, 5, 6, 7, 8, 9, or all 10 pairs of features having a correlation of at least 0.6 is excluded from the model. In some embodiments, an excluded feature is the feature in a pair of highly correlated features having the lower regression coefficient reported in Table 23. For instance, with reference to Table 24, the feature having the lower regression coefficient in each highly correlated pair (e.g., corresponding to a correlation of at least 0.6) are:
Pair 1 = DSG1
Pair 2 = ZFR2
Pair 3 = RNF212
Pair 4 = SYCP2
Pair 5 = ZFR2
Pair 6 = MYO3A
Pair 7 = SYCP2
Pair 8 = DSG1
Pair 9 = KCNS1
Pair 10 = ZFR2
Accordingly, in some embodiments, one or more of DSG1, ZFR2, RNF212, SYCP2, MYO3A, and KCNS1 are excluded from the features set on the basis that they are the least informative feature in a pair of highly correlated features.

However, in some embodiments, this selection process does not allow both features of a highly correlated pair of features to be excluded from the feature set, e.g., on the basis that both genes are the least informative feature in at least one of the highly correlated pairs of features. Thus, in some embodiments, one or more of SYCP2, MYO3A,and KCNS1 are not excluded from the feature set. Similarly, in some embodiments, this selection process does not allow highly informative features, e.g., features with regression coefficients of at least 0.5, to be excluded from the feature set. Thus, in some embodiments, one or both of RNF212 and KCNS1 are not excluded from the feature set.

Accordingly, in one embodiment, the feature set includes abundance values for at least KRT86, CRISPLD1, SESN3, DAMTS20, IRX1, SMC1B, CDKN2A, EFNB3, CXCL14, MKRN3, SYCP2, MYL1, MYO3A,RNASE10, GALNT13, C19orf26, MUC4, PCDHGB1, CCND1, LCE1F, and KCNS1.

Similarly, in one embodiment, the feature set includes abundance values for at least KRT86, CRISPLD1, SESN3, DAMTS20, IRX1, SMC1B, CDKN2A, EFNB3, CXCL14, RNF212, MKRN3, MYL1, RNASE10, GALNT13, C19orf26, MUC4, PCDHGB1, CCND1, LCE1F, and KCNS1.

Similarly, in one embodiment, the feature set includes abundance values for at least KRT86, CRISPLD1, SESN3, DAMTS20, IRX1, SMC1B, CDKN2A, EFNB3, CXCL14, RNF212, MKRN3, SYCP2, MYL1, MYO3A,RNASE10, GALNT13, C19orf26, MUC4, PCDHGB1, CCND1, LCE1F, and KCNS1.

In some embodiments, as described above referring to FIG. 7, and in conjunction with the description of FIG. 2 in U.S. Pat. Application Publication No. 2020/0273576, the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm. In some embodiments, the classifier was trained according to a methodology described above, in reference to FIG. 7, and in conjunction with the description of FIG. 2 in U.S. Pat. Application Publication No. 2020/0273576.

In some embodiments, the classifier has a specificity of at least 70% and a sensitivity of at least 70% for a validation data set of at least 50 data constructs, e.g., where none of the data constructs in the validation data set were used in the training of the classifier. In some embodiments, the classifier has a specificity of at least 75% and a sensitivity of at least 75% for a validation data set of at least 50 data constructs, e.g., where none of the data constructs in the validation data set were used in the training of the classifier. In some embodiments, the classifier has a specificity of at least 80% and a sensitivity of at least 80% for a validation data set of at least 50 data constructs, e.g., where none of the data constructs in the validation data set were used in the training of the classifier. In some embodiments, the classifier has a specificity of at least 85% and a sensitivity of at least 85% for a validation data set of at least 50 data constructs, e.g., where none of the data constructs in the validation data set were used in the training of the classifier. In some embodiments, the classifier has a specificity of at least 90% and a sensitivity of at least 90% for a validation data set of at least 50 data constructs, e.g., where none of the data constructs in the validation data set were used in the training of the classifier. In some embodiments, the classifier has a specificity of at least 95% and a sensitivity of at least 95% for a validation data set of at least 50 data constructs, e.g., where none of the data constructs in the validation data set were used in the training of the classifier. In some embodiments, the classifier has a specificity of at least 70%, 75%, 80%, 85%, 90%, 95%, 96%, 97%, 98%, 99%, or higher for a validation data set of at least 50 data constructs. In some embodiments, the classifier has a sensitivity of at least 70%, 75%, 80%, 85%, 90%, 95%, 96%, 97%, 98%, 99%, or higher for a validation data set of at least 50 data constructs.

In some embodiments, the classifier has a specificity of at least 70% and a sensitivity of at least 70% for a validation data set of at least 100 data constructs, e.g., where none of the data constructs in the validation data set were used in the training of the classifier. In some embodiments, the classifier has a specificity of at least 75% and a sensitivity of at least 75% for a validation data set of at least 100 data constructs, e.g., where none of the data constructs in the validation data set were used in the training of the classifier. In some embodiments, the classifier has a specificity of at least 80% and a sensitivity of at least 80% for a validation data set of at least 100 data constructs, e.g., where none of the data constructs in the validation data set were used in the training of the classifier. In some embodiments, the classifier has a specificity of at least 85% and a sensitivity of at least 85% for a validation data set of at least 100 data constructs, e.g., where none of the data constructs in the validation data set were used in the training of the classifier. In some embodiments, the classifier has a specificity of at least 90% and a sensitivity of at least 90% for a validation data set of at least 100 data constructs, e.g., where none of the data constructs in the validation data set were used in the training of the classifier. In some embodiments, the classifier has a specificity of at least 95% and a sensitivity of at least 95% for a validation data set of at least 100 data constructs, e.g., where none of the data constructs in the validation data set were used in the training of the classifier. In some embodiments, the classifier has a specificity of at least 70%, 75%, 80%, 85%, 90%, 95%, 96%, 97%, 98%, 99%, or higher for a validation data set of at least 100 data constructs. In some embodiments, the classifier has a sensitivity of at least 70%, 75%, 80%, 85%, 90%, 95%, 96%, 97%, 98%, 99%, or higher for a validation data set of at least 100 data constructs.

In some embodiments, the method further includes assigning therapy and/or administering therapy to the subject based on the classification of the cancer condition, e.g., based on whether or not the subject’s cancer is associated with an HPV viral infection.

Accordingly, in one embodiment, a method is provided for treating cervical cancer in a human cancer patient. The method includes determining whether the human cancer patient is infected with a human papillomavirus (HPV) oncogenic virus by obtaining a dataset for the human cancer patient, the dataset including a plurality of abundance values where each respective abundance value in the plurality of abundance values quantifies a level of expression of a corresponding gene, in a plurality of genes, and the plurality of genes includes at least five genes selected from the genes listed in Table 21. The method then includes inputting the dataset to a classifier trained to discriminate between at least a first cervical cancer condition associated with HPV infection and a second cervical cancer condition associated with an HPV-free status based on the abundance values of the plurality of genes, in a cancerous tissue of the subject. In some embodiments, the classifier is trained according to a methodology described above, referring to FIG. 7, and in conjunction with the description of FIG. 2 in U.S. Pat. Application Publication No. 2020/0273576. The method then includes treating the cervical cancer. When the classifier result indicates that the human cancer patient is infected with an HPV oncogenic virus, administering a first therapy tailored for treatment of cervical cancer associated with an HPV infection. When the classifier result indicates that the human cancer patient is not infected with an HPV oncogenic virus, administering a second therapy tailored for treatment of cervical cancer not associated with an HPV infection.

In some embodiments, the plurality of genes includes at least ten of the genes listed in Table 21. In some embodiments, the plurality of genes includes at least fifteen of the genes listed in Table 21. In some embodiments, the plurality of genes includes at least twenty of the genes listed in Table 21. In some embodiments, the plurality of genes includes all of the genes listed in Table 21. In some embodiment, the plurality of genes includes one or more genes that are not listed in Table 21, e.g., 1, 2, 3, 4, 5, 6, 7, 8, 9, 10, or more of the genes not listed in Table 21. In some embodiments, the plurality of genes includes no more than 20 genes. In some embodiments, the plurality of genes includes no more than 25 genes. In some embodiments, the plurality of genes includes no more than 50 genes. In some embodiments, the plurality of genes includes no more than 10, 15, 20, 25, 30, 35, 40, 50, 60, 70, 80, 90, 100, 125, 150, 175, 200, 250, or 300 genes.

In some embodiments, the dataset also includes a variant allele count for one or more alleles at one or more loci in the genome of the cancerous tissue from the subject. In some embodiments, the variant allele count is either 1, representing a state in which the subject carries the variant allele, or 0, representing a state in which the subject does not carry the variant allele. In some embodiments, the variant allele is a somatic variant, originating from the germ line of the subject. In some embodiments, the variant allele is a cancer-derived variant, originating from the cancerous tissue. In some embodiments, the variant allele is located in the TP53 (ENSG00000141510) or CDKN2A (ENSG00000147889) gene.

In some embodiments, as described above referring to FIG. 7, and in conjunction with the description of FIG. 2 in U.S. Pat. Application Publication No. 2020/0273576, the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm. In some embodiments, the classifier was trained according to a methodology described above, in reference to FIG. 7, and in conjunction with the description of FIG. 2 in U.S. Pat. Application Publication No. 2020/0273576.

In some embodiments, the first therapy tailored for treatment of cervical cancer associated with an HPV infection is a therapeutic vaccine. In some embodiments, the therapeutic vaccine is selected from axalimogene filolisbac (Advaxis), TG4001 (Transgene), GX-188E (Genexine), VGX-3100 (Inovio), MEDI-0457 (Inovio), INO-3106 (Inovio), TA-CIN (Cancer Research Technology), TA-HPV (Cancer Research Technology), ISA-101 (Isa), and PepCan (University of Arkansas).

In some embodiments, the first therapy tailored for treatment of cervical cancer associated with an HPV infection is an adoptive cell therapy. In some embodiments, adoptive cell therapy includes the administration of HPV-specific T cells, for example, as described for clinical trial ID NCT02379520 or NCT03197025 (Baylor College of Medicine).

In some embodiments, the first therapy tailored for treatment of cervical cancer associated with an HPV infection is an immune checkpoint inhibitor. In some embodiments, the immune checkpoint inhibitor is nivolumab (Bristol-Myers Squibb).

In some embodiments, the first therapy tailored for treatment of cervical cancer associated with an HPV infection is a PI3K inhibitor. In some embodiments, the PI3K inhibitor is AMG319 (Amgen) or BKM120 (Novartis).

Similarly, in one embodiment, a method is provided for treating head and neck cancer in a human cancer patient. The method includes determining whether the human cancer patient is infected with a human papillomavirus (HPV) oncogenic virus by obtaining a dataset for the human cancer patient, the dataset including a plurality of abundance values where each respective abundance value in the plurality of abundance values quantifies a level of expression of a corresponding gene, in a plurality of genes, and the plurality of genes includes at least five genes selected from the genes listed in Table 21. The method then includes inputting the dataset to a classifier trained to discriminate between at least a first head and neck cancer condition associated with HPV infection and a second head and neck cancer condition associated with an HPV-free status based on the abundance values of the plurality of genes, in a cancerous tissue of the subject. In some embodiments, the classifier is trained according to a methodology described above, referring to FIG. 7, and in conjunction with the description of FIG. 2 in U.S. Pat. Application Publication No. 2020/0273576. The method then includes treating the head and neck cancer. When the classifier result indicates that the human cancer patient is infected with an HPV oncogenic virus, the method includes administering a first therapy tailored for treatment of head and neck cancer associated with an HPV infection. When the classifier result indicates that the human cancer patient is not infected with an HPV oncogenic virus, the method includes administering a second therapy tailored for treatment of head and neck cancer not associated with an HPV infection.

In some embodiments, the plurality of genes includes at least ten of the genes listed in Table 21. In some embodiments, the plurality of genes includes at least fifteen of the genes listed in Table 21. In some embodiments, the plurality of genes includes at least twenty of the genes listed in Table 21. In some embodiments, the plurality of genes includes all of the genes listed in Table 21. In some embodiment, the plurality of genes includes one or more genes that are not listed in Table 21, e.g., 1, 2, 3, 4, 5, 6, 7, 8, 9, 10, or more of the genes not listed in Table 21. In some embodiments, the plurality of genes includes no more than 20 genes. In some embodiments, the plurality of genes includes no more than 25 genes. In some embodiments, the plurality of genes includes no more than 50 genes. In some embodiments, the plurality of genes includes no more than 10, 15, 20, 25, 30, 35, 40, 50, 60, 70, 80, 90, 100, 125, 150, 175, 200, 250, or 300 genes.

In some embodiments, the dataset also includes a variant allele count for one or more alleles at one or more loci in the genome of the cancerous tissue from the subject. In some embodiments, the variant allele count is either 1, representing a state in which the subject carries the variant allele, or 0, representing a state in which the subject does not carry the variant allele. In some embodiments, the variant allele is a somatic variant, originating from the germ line of the subject. In some embodiments, the variant allele is a cancer-derived variant, originating from the cancerous tissue. In some embodiments, the variant allele is located in the TP53 (ENSG00000141510) or CDKN2A (ENSG00000147889) gene.

In some embodiments, as described above referring to FIG. 7, and in conjunction with the description of FIG. 2 in U.S. Pat. Application Publication No. 2020/0273576, the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm. In some embodiments, the classifier was trained according to a methodology described above, in reference to FIG. 7, and in conjunction with the description of FIG. 2 in U.S. Pat. Application Publication No. 2020/0273576.

In some embodiments, the first therapy tailored for treatment of head and neck cancer associated with an HPV infection is a therapeutic vaccine. In some embodiments, the therapeutic vaccine is selected from axalimogene filolisbac (Advaxis), TG4001 (Transgene), GX-188E (Genexine), VGX-3100 (Inovio), MEDI-0457 (Inovio), INO-3106 (Inovio), TA-CIN (Cancer Research Technology), TA-HPV (Cancer Research Technology), ISA-101 (Isa), and PepCan (University of Arkansas).

In some embodiments, the first therapy tailored for treatment of head and neck cancer associated with an HPV infection is an adoptive cell therapy. In some embodiments, adoptive cell therapy includes the administration of HPV-specific T cells, for example, as described for clinical trial ID NCT02379520 or NCT03197025 (Baylor College of Medicine).

In some embodiments, the first therapy tailored for treatment of head and neck cancer associated with an HPV infection is an immune checkpoint inhibitor. In some embodiments, the immune checkpoint inhibitor is nivolumab (Bristol-Myers Squibb).

In some embodiments, the first therapy tailored for treatment of head and neck cancer associated with an HPV infection is a PI3K inhibitor. In some embodiments, the PI3K inhibitor is AMG319 (Amgen) or BKM120 (Novartis).

### HPV Probe Sets

In some embodiments, the present disclosure provides probes for binding, enriching, and or detecting nucleic acid molecules, e.g., mRNA transcripts that are isolated from a cancerous tissue sample from a subject and/or cDNA molecules prepared from those mRNA transcripts, that are informative of whether the subject has a first cancer condition associated with an HPV oncogenic viral infection or a second cancer condition that is not associated with an HPV oncogenic viral infection. Generally, the probes include DNA, RNA, or a modified nucleic acid structure with a base sequence that is complementary of a nucleic acid molecule of interest. Accordingly, when the probe is designed to hybridize to an mRNA molecule isolated from the cancerous tissue, the probe will include a nucleic acid sequence that is complementary to the coding strand of the gene from which the transcript originated, i.e., the probe will include an antisense sequence of the gene. However, when the probe is designed to hybridize to a cDNA molecule, the probe can contain either a sequence that is complementary to the coding sequence of the gene of interest (an antisense sequence) or a sequence that is identical to the coding sequence of the gene of interest (a sense sequence), because the molecules in the cDNA library are double stranded.

In some embodiments, the probes include additional nucleic acid sequences that do not share any homology to the gene sequence of interest. For example, in some embodiments, the probes also include nucleic acid sequences containing an identifier sequence, e.g., a unique molecular identifier (UMI), e.g., that is unique to a particular cancerous tissue sample or cancer patient. Examples of identifier sequences are described, for example, in Kivioja et al., 2011, Nat. Methods 9(1), pp. 72-74 and Islam et al., 2014, Nat. Methods 11(2), pp. 163-66, the contents of which are hereby incorporated herein by reference, in their entireties, for all purposes. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probe includes a non-nucleic acid affinity moiety covalently attached to nucleic acid molecule that is complementary to the gene of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest.

Accordingly, in one embodiment, the disclosure provides a plurality of nucleic acid probes for discriminating between a first cancer condition and a second cancer condition in a human subject, where the first cancer condition is associated with infection by a human papillomavirus (HPV) oncogenic virus and the second cancer condition is associated with an HPV-free status. The plurality of nucleic acid probes includes at least five nucleic acid probes, and each of the at least five nucleic acid probes includes a respective nucleic acid sequence that is identical or complementary to at least 10 consecutive bases of an RNA transcript of a different respective gene selected from the genes listed in Table 21.

In some embodiments, the plurality of nucleic acid probes includes at least ten probes with sequences that are complementary to or identical to sequences from different genes listed in Table 21. In some embodiments, the plurality of nucleic acid probes includes at least fifteen probes with sequences that are complementary to or identical to sequences from different genes listed in Table 21. In some embodiments, the plurality of nucleic acid probes includes at least twenty probes with sequences that are complementary to or identical to sequences from different genes listed in Table 21. In some embodiments, the plurality of nucleic acid probes includes probes with sequences that are complementary to or identical to sequences from all of the genes listed in Table 21. In some embodiments, the plurality of nucleic acid probes includes 2, 3, 4, 5, 6, 7, 8, 9, 10, 11, 12, 13, 14, 15, 16, 17, 18, 19, 20, 21, 22, 23, or 24 probes with sequences that are complementary to or identical to sequences from different genes listed in Table 21.

In some embodiments, the plurality of nucleic acid probes includes one or more probes that bind to a sequence of a gene that is not listed in Table 21. In some embodiments, the plurality of nucleic acid probes includes at least 2, 3, 4, 5, 6, 7, 8, 9, 10, or more probes that bind to a sequence of a gene that is not listed in Table 21. In some embodiments, the plurality of nucleic acid probes includes probes with sequences that bind to no more than 20 genes. In some embodiments, the plurality of nucleic acid probes includes probes with sequences that bind to no more than 25 genes. In some embodiments, the plurality of nucleic acid probes includes probes with sequences that bind to no more than 50 genes. In some embodiments, the plurality of nucleic acid probes includes probes with sequences that bind to no more than 10, 15, 20, 25, 30, 35, 40, 50, 60, 70, 80, 90, 100, 125, 150, 175, 200, 250, or 300 genes.

In some embodiments, each probe in the plurality of probes includes a nucleic acid sequence that is identical or complementary to at least 15 consecutive bases of an RNA transcript of interest, e.g., a transcript from a gene listed in Table 21. In some embodiments, each probe in the plurality of probes includes a nucleic acid sequence that is identical or complementary to at least 30 consecutive bases of an RNA transcript of interest, e.g., a transcript from a gene listed in Table 21. In some embodiments, each probe in the plurality of probes includes a nucleic acid sequence that is identical or complementary to at least 50 consecutive bases of an RNA transcript of interest, e.g., a transcript from a gene listed in Table 21. In some embodiments, each probe in the plurality of probes includes a nucleic acid sequence that is identical or complementary to at least 10, 15, 20, 25, 30, 35, 40, 50, 60, 70, 80, 90, 100, 125, 150, 175, 200, or more consecutive bases of an RNA transcript of interest, e.g., a transcript from a gene listed in Table 21.

### EBV Oncogenic Viral Infections

In some embodiments, the methods described herein relate to classification and/or treatment of cancers known to be associated with an Epstein-Barr virus (EBV) infection. As reported in Example 4, below, the twenty-four genes listed in Table 22, and shown in FIG. 5B, were found to be differentially expressed in at least eight of the ten training sets formed from expression data of gastric cancer with known EBV statuses in The Cancer Genome Atlas (TCGA). Accordingly, in some embodiments the expression levels of one or more of the genes listed in Table 22 are used for the classification of gastric cancer as either associated with an EBV infection or not associated with an EBV infection. In some embodiments, expression levels of at least 2, 3, 4, 5, 6, 7, 8, or all 9 of the genes listed in Table 22 are used for the classification of gastric cancer as either associated with an EBV infection or not associated with an EBV infection.

TABLE 22

Genes found to be differentially expressed in at least 80% of the gastric cancer training sets derived from the TCGA database

| | |
|---|---|
| ENSEMBL ACCESSION ID | GENE NAME |
| ENSG00000111319 | SCNN1A |
| ENSG00000113722 | CDX1 |
| ENSG00000124249 | KCNK15 |
| ENSG00000126583 | PRKCG |
| ENSG00000135480 | KRT7 |
| ENSG00000145506 | NKD2 |
| ENSG00000151025 | GPR158 |
| ENSG00000165215 | CLDN3 |
| ENSG00000176083 | ZNF683 |

In one embodiment, a method is provided for discriminating between a first cancer condition and a second cancer condition in a human subject, wherein the first cancer condition is associated with infection by an Epstein-Barr virus (EBV) oncogenic virus and the second cancer condition is associated with an EBV-free status. The method includes obtaining a dataset for the subject, e.g., as described above with reference to FIG. 8. The dataset includes a plurality of abundance values from the subject, where each respective abundance value in the plurality of abundance values quantifies a level of expression of a corresponding gene, in a plurality of genes, in a cancerous tissue from the subject. In some embodiments, the plurality of genes includes at least five genes selected from the genes listed in Table 22. The method then includes inputting the dataset to a classifier trained to discriminate between at least the first cancer condition and the second cancer condition based on the abundance values of the plurality of genes. In some embodiments, the classifier is trained in accordance with any of the methodologies described above, with respect to FIG. 7, and in conjunction with the description of FIG. 2 in U.S. Pat. Application Publication No. 2020/0273576.

In some embodiments, the plurality of genes includes all of the genes listed in Table 22. In some embodiment, the plurality of genes includes one or more genes that are not listed in Table 22, e.g., 1, 2, 3, 4, 5, 6, 7, 8, 9, 10, or more of the genes not listed in Table 22. In some embodiments, the plurality of genes includes no more than 20 genes. In some embodiments, the plurality of genes includes no more than 25 genes. In some embodiments, the plurality of genes includes no more than 50 genes. In some embodiments, the plurality of genes includes no more than 10, 15, 20, 25, 30, 35, 40, 50, 60, 70, 80, 90, 100, 125, 150, 175, 200, 250, or 300 genes.

In some embodiments, the dataset also includes a variant allele count for one or more alleles at one or more loci in the genome of the cancerous tissue from the subject. In some embodiments, the variant allele count is either 1, representing a state in which the subject carries the variant allele, or 0, representing a state in which the subject does not carry the variant allele. In some embodiments, the variant allele is a somatic variant, originating from the germ line of the subject. In some embodiments, the variant allele is a cancer-derived variant, originating from the cancerous tissue. In some embodiments, the variant allele is located in the TP53 (ENSG00000141510) or PIK3CA (ENSG00000121879) gene.

In some embodiments, the classifier is trained for determining the EBV status of a test subject having an EBV-associated cancer selected from Burkitt’s lymphoma, sinonasal angiocentric T-cell lymphoma, non-Hodgkin’s lymphoma, Hodgkin’s lymphoma, nasopharyngeal carcinoma, and gastric cancer. In some embodiments, the classifier is trained for determining the EBV status of a test patient having a specific EBV-associated cancer, e.g., Burkitt’s lymphoma, sinonasal angiocentric T-cell lymphoma, non-Hodgkin’s lymphoma, Hodgkin’s lymphoma, nasopharyngeal carcinoma, or gastric cancer. However, as classifier training is generally improved by increasing the size of the training dataset, in some embodiments, the classifier is trained against data from patients that have two or more types of EBV-associated cancers, e.g., two, three, four, five, or all six of Burkitt’s lymphoma, sinonasal angiocentric T-cell lymphoma, non-Hodgkin’s lymphoma, Hodgkin’s lymphoma, nasopharyngeal carcinoma, and gastric cancer. In a particular embodiment, exemplified by Example 4, the classifier is trained against patients having gastric cancer. However, in some embodiments, a classifier trained against patients having one or more types of EBV-associated cancer is useful for determining the EBV status of a patient having a different type of EBV-associated cancer.

In some embodiments, the features of the classifier include abundance values for a plurality of genes selected from those listed in Table 22, e.g., SCNN1A, CDX1, KCNK15, PRKCG, KRT7, NKD2, GPR158, CLDN3, and ZNF683. As reported below, e.g., in reference to Example 4, these nine genes were found to be differentially expressed, dependent upon the EBV status of the subject, in at least 80% of the gastric cancer training sets in The Cancer Genome Atlas (TCGA). However, the skilled artisan will appreciate that, is some instances, the use of different training data sets may yield different results, e.g., one or more of these genes may not be informative in at least 80% of training folds and/or one or more genes found not to be informative in at least 80% of training folds in the study reported in Example 4 may be informative. These differences may arise, for example, when different criteria are used to select the training population, e.g., different inclusion and/or exclusion criteria such as cancer type, personal characteristics (e.g., age, gender, ethnicity, family history, smoking status, etc.), or simply by using a smaller or larger data set.

Accordingly, in some embodiments, the features of the classifier include at least five of the genes listed in Table 22. In some embodiments, the features of the classifier include at least six of the genes listed in Table 22. In some embodiments, the features of the classifier include at least seven of the genes listed in Table 22. In some embodiments, the features of the classifier include at least eight of the genes listed in Table 22. In some embodiments, the features of the classifier include all nine of the genes listed in Table 22. Further, in some embodiments, the features of the classifier also include the abundance values for one or more genes not listed in Table 22. In some embodiments, the features of the classifier include the abundance value for 1, 2, 3, 4, 5, 6, 7, 8, 9, 10 or more genes not listed in Table 22. In some embodiments, the features of the classifier include the abundance values for 1-10 genes not listed in Table 22. In some embodiments, the features of the classifier include 1-5 genes not listed in Table 22. In other embodiments, the features of the classifier do not include the abundance values for any genes not listed in Table 22.

Further, the skilled artisan will also appreciate that some features, e.g., abundance values for a particular gene, will be more informative than other features in a particular classifier. One measure of the predictive power of respective features in a classifier based on multiple features is the regression coefficient calculated for the features during training of the model. Regression coefficients describe the relationship between each feature and the response of the model. The coefficient value represents the mean change in the response given a one-unit increase in the feature value. As such, at least for variables of the same type, the magnitude, e.g., absolute value, of a regression coefficient is correlated with the importance of the feature in the model. That is, the higher the magnitude of the regression coefficient, the more important the variable is to the model. For instance, as reported in Example 4, in a particular support vector machine (SVM) classifier trained against the abundance values of all nine of the genes listed in Table 22, as well as a variant allele status for the TP53 and PIK3CA genes, only four of the nine genes had regression coefficients with magnitudes of at least 0.75-SCNN1A (-1.26), KCNK15 (-1.04), KRT7 (-0.94), and CLDN3 (-1.68).

As such, the skilled artisan may select a feature set that includes less than all of the genes listed in Table 22 based, at least in part, upon the importance of the respective features in one or more classification models. For instance, in some embodiments, one or more genes with lower predictive power in a classification model may be left out during classifier training. For example, in some embodiments, the features of the classifier include at least the gene expression features listed in Table 23 with a regression coefficient of at least 0.75, e.g., SCNN1A (-1.26), KCNK15 (-1.04), KRT7 (-0.94), and CLDN3 (-1.68). In some embodiments, the features of the classifier include at least the gene expression features listed in Table 23 with a regression coefficient of at least 0.6.

Similarly, the size of the feature set may be affected by which features are included and/or excluded. For instance, in some embodiments, if particular features having high predictive power are included in a classification model, fewer total features may be included in the model. For instance, in some embodiments, if the abundance values for SCNN1A, KCNK15, KRT7, and CLDN3 are included in the model, the abundance values for no more than one of the other genes listed in Table 22 need to be included in the model. Accordingly, in some embodiments, the features of the classifier include abundance values for SCNN1A, KCNK15, KRT7, and CLDN3, and at least one other gene listed in Table 22. In some embodiments, the features of the classifier include abundance values for SCNN1A, KCNK15, KRT7, and CLDN3, and at least two other genes listed in Table 22. In some embodiments, the features of the classifier include abundance values for SCNN1A, KCNK15, KRT7, and CLDN3, and at least three other genes listed in Table 22. In some embodiments, the features of the classifier include abundance values for SCNN1A, KCNK15, KRT7, and CLDN3, and at least four other genes listed in Table 22.

Similarly, in some embodiments, if features having high predictive power are excluded from the classification model, more of the other features may be included in the model. For instance, in some embodiments, if the abundance values for one or more of SCNN1A, KCNK15, KRT7, and CLDN3 are not included in the model, the abundance values for at least four of the other genes listed in Table 22 are included in the model. In some embodiments, if the abundance values for one or more of SCNN1A, KCNK15, KRT7, and CLDN3 are not included in the model, the abundance values for all five of the other genes listed in Table 22 are included in the model.

Of course, other metrics are also available for evaluating the importance of a feature in a model, such as standardized regression coefficients and change in R-squared when the comparing the output of a model having the feature to the output of a model that is identical except that it lacks the feature.

When selecting a feature set, the skilled artisan will also consider the degree to which features are correlated to each other. Correlation is a statistical measure of how linearly dependent two variables are upon each other. As such, two correlated features provide duplicative information to a predictive model, which can be detrimental to a classifier. As such, there are several reasons why a correlated feature may be excluded from a model. For instance, removing a correlated feature will make the algorithm faster, as the larger the number of features in a classifier the more computations that need to be made. Removing a correlated feature may also remove harmful bias, arising from the correlation, from a model. Finally, removing a correlated feature may make the model more interpretable. As such, the skilled artisan may select a feature set that includes less than all of the genes listed in Table 21 based, at least in part, upon the correlation between respective features in one or more classification models. For example, statistical analysis of the SVM model trained in Example 4 revealed that the gene expression values for ENSG00000135480 (KRT7) and ENSG00000124249 (KCNK15) were highly correlated (0.650). Accordingly, in some embodiments, the abundance value for one of KRT7 and KCNK15 are excluded from the feature set.

For example, in one embodiment, the feature set includes abundance values for at least SCNN1A, CDX1, KCNK15, PRKCG, NKD2, GPR158, CLDN3, and ZNF683. In another embodiment, the feature set includes abundance values for at least SCNN1A, CDX1, PRKCG, KRT7, NKD2, GPR158, CLDN3, and ZNF683.

In some embodiments, as described above referring to FIG. 7, and in conjunction with the description of FIG. 2 in U.S. Pat. Application Publication No. 2020/0273576, the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm. In some embodiments, the classifier was trained according to a methodology described above, in reference to FIG. 7, and in conjunction with the description of FIG. 2 in U.S. Pat. Application Publication No. 2020/0273576.

In some embodiments, the classifier has a specificity of at least 70% and a sensitivity of at least 70% for a validation data set of at least 50 data constructs, e.g., where none of the data constructs in the validation data set were used in the training of the classifier. In some embodiments, the classifier has a specificity of at least 75% and a sensitivity of at least 75% for a validation data set of at least 50 data constructs, e.g., where none of the data constructs in the validation data set were used in the training of the classifier. In some embodiments, the classifier has a specificity of at least 80% and a sensitivity of at least 80% for a validation data set of at least 50 data constructs, e.g., where none of the data constructs in the validation data set were used in the training of the classifier. In some embodiments, the classifier has a specificity of at least 85% and a sensitivity of at least 85% for a validation data set of at least 50 data constructs, e.g., where none of the data constructs in the validation data set were used in the training of the classifier. In some embodiments, the classifier has a specificity of at least 90% and a sensitivity of at least 90% for a validation data set of at least 50 data constructs, e.g., where none of the data constructs in the validation data set were used in the training of the classifier. In some embodiments, the classifier has a specificity of at least 95% and a sensitivity of at least 95% for a validation data set of at least 50 data constructs, e.g., where none of the data constructs in the validation data set were used in the training of the classifier. In some embodiments, the classifier has a specificity of at least 70%, 75%, 80%, 85%, 90%, 95%, 96%, 97%, 98%, 99%, or higher for a validation data set of at least 50 data constructs. In some embodiments, the classifier has a sensitivity of at least 70%, 75%, 80%, 85%, 90%, 95%, 96%, 97%, 98%, 99%, or higher for a validation data set of at least 50 data constructs.

In some embodiments, the classifier has a specificity of at least 70% and a sensitivity of at least 70% for a validation data set of at least 100 data constructs, e.g., where none of the data constructs in the validation data set were used in the training of the classifier. In some embodiments, the classifier has a specificity of at least 75% and a sensitivity of at least 75% for a validation data set of at least 100 data constructs, e.g., where none of the data constructs in the validation data set were used in the training of the classifier. In some embodiments, the classifier has a specificity of at least 80% and a sensitivity of at least 80% for a validation data set of at least 100 data constructs, e.g., where none of the data constructs in the validation data set were used in the training of the classifier. In some embodiments, the classifier has a specificity of at least 85% and a sensitivity of at least 85% for a validation data set of at least 100 data constructs, e.g., where none of the data constructs in the validation data set were used in the training of the classifier. In some embodiments, the classifier has a specificity of at least 90% and a sensitivity of at least 90% for a validation data set of at least 100 data constructs, e.g., where none of the data constructs in the validation data set were used in the training of the classifier. In some embodiments, the classifier has a specificity of at least 95% and a sensitivity of at least 95% for a validation data set of at least 100 data constructs, e.g., where none of the data constructs in the validation data set were used in the training of the classifier. In some embodiments, the classifier has a specificity of at least 70%, 75%, 80%, 85%, 90%, 95%, 96%, 97%, 98%, 99%, or higher for a validation data set of at least 100 data constructs. In some embodiments, the classifier has a sensitivity of at least 70%, 75%, 80%, 85%, 90%, 95%, 96%, 97%, 98%, 99%, or higher for a validation data set of at least 100 data constructs.

In some embodiments, the method further includes assigning therapy and/or administering therapy to the subject based on the classification of the cancer condition, e.g., based on whether or not the subject’s cancer is associated with an EBV viral infection.

Accordingly, in one embodiment, a method is provided for treating gastric cancer in a human cancer patient. The method includes determining whether the human cancer patient is infected with an Epstein-Barr virus (EBV) oncogenic virus by obtaining a dataset for the human cancer patient, the dataset including a plurality of abundance values where each respective abundance value in the plurality of abundance values quantifies a level of expression of a corresponding gene, in a plurality of genes, and the plurality of genes includes at least five genes selected from the genes listed in Table 22. The method then includes inputting the dataset to a classifier trained to discriminate between at least a first gastric cancer condition associated with an EBV infection and a second gastric cancer condition associated with an EBV-free status based on the abundance values of the plurality of genes, in a cancerous tissue of the subject. In some embodiments, the classifier is trained according to a methodology described above, referring to FIG. 7, and in conjunction with the description of FIG. 2 in U.S. Pat. Application Publication No. 2020/0273576. The method then includes treating the gastric cancer. When the classifier result indicates that the human cancer patient is infected with an EBV oncogenic virus, administering a first therapy tailored for treatment of gastric cancer associated with an EBV infection. When the classifier result indicates that the human cancer patient is not infected with an EBV oncogenic virus, administering a second therapy tailored for treatment of gastric cancer not associated with an EBV infection.

In some embodiments, the plurality of genes includes all of the genes listed in Table 22. In some embodiment, the plurality of genes includes one or more genes that are not listed in Table 22, e.g., 1, 2, 3, 4, 5, 6, 7, 8, 9, 10, or more of the genes not listed in Table 22. In some embodiments, the plurality of genes includes no more than 20 genes. In some embodiments, the plurality of genes includes no more than 25 genes. In some embodiments, the plurality of genes includes no more than 50 genes. In some embodiments, the plurality of genes includes no more than 10, 15, 20, 25, 30, 35, 40, 50, 60, 70, 80, 90, 100, 125, 150, 175, 200, 250, or 300 genes.

In some embodiments, the dataset also includes a variant allele count for one or more alleles at one or more loci in the genome of the cancerous tissue from the subject. In some embodiments, the variant allele count is either 1, representing a state in which the subject carries the variant allele, or 0, representing a state in which the subject does not carry the variant allele. In some embodiments, the variant allele is a somatic variant, originating from the germ line of the subject. In some embodiments, the variant allele is a cancer-derived variant, originating from the cancerous tissue. In some embodiments, the variant allele is located in the TP53 (ENSG00000141510) or PIK3CA (ENSG00000121879) gene.

In some embodiments, as described above referring to FIG. 7, and in conjunction with the description of FIG. 2 in U.S. Pat. Application Publication No. 2020/0273576, the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm. In some embodiments, the classifier was trained according to a methodology described above, in reference to FIG. 7, and in conjunction with the description of FIG. 2 in U.S. Pat. Application Publication No. 2020/0273576.

In some embodiments, the first therapy tailored for treatment of gastric cancer associated with an EBV infection is an adoptive cell therapy. In some embodiments, the adoptive cell therapy includes is ATA 129 (Atara), EBVST (Tessa), or CMD-003 (Cell Medica).

In some embodiments, the first therapy tailored for treatment of gastric cancer associated with an EBV infection is an immune checkpoint inhibitor. In some embodiments, the immune checkpoint inhibitor is Pembrozilumab (Merck) or nivolumab (Bristol-Myers Squibb).

In some embodiments, the first therapy tailored for treatment of gastric cancer associated with an EBV infection is a BTK inhibitor. In some embodiments, the BTK inhibitor is ibrutinib (Pharmacyclics).

### EBV Model Probe Sets

In some embodiments, the present disclosure provides probes for binding, enriching, and or detecting nucleic acid molecules, e.g., mRNA transcripts that are isolated from a cancerous tissue sample from a subject and/or cDNA molecules prepared from those mRNA transcripts, that are informative of whether the subject has a first cancer condition associated with an EBV oncogenic viral infection or a second cancer condition that is not associated with an EBV oncogenic viral infection. Generally, the probes include DNA, RNA, or a modified nucleic acid structure with a base sequence that is complementary of a nucleic acid molecule of interest. Accordingly, when the probe is designed to hybridize to an mRNA molecule isolated from the cancerous tissue, the probe will include a nucleic acid sequence that is complementary to the coding strand of the gene from which the transcript originated, e.g., the probe will include an antisense sequence of the gene. However, when the probe is designed to hybridize to a cDNA molecule, the probe can contain either a sequence that is complementary to the coding sequence of the gene of interest (an antisense sequence) or a sequence that is identical to the coding sequence of the gene of interest (a sense sequence), because the molecules in the cDNA library are double stranded.

In some embodiments, the probes include additional nucleic acid sequences that do not share any homology to the gene sequence of interest. For example, in some embodiments, the probes also include nucleic acid sequences containing an identifier sequence, e.g., a unique molecular identifier (UMI), e.g., that is unique to a particular cancerous tissue sample or cancer patient. Examples of identifier sequences are described, for example, in Kivioja et al., 2011, Nat. Methods 9(1):72-74 and Islam et al., 2014, Nat. Methods 11(2), pp. 163-66, the contents of which are incorporated herein by reference, in their entireties, for all purposes. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probe includes a non-nucleic acid affinity moiety covalently attached to nucleic acid molecule that is complementary to the gene of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, e.g., a dip-stick or magnetic bead, for recovering the nucleic acid of interest.

Accordingly, in one embodiment, the disclosure provides a plurality of nucleic acid probes for discriminating between a first cancer condition and a second cancer condition in a human subject, where the first cancer condition is associated with infection by an Epstein-Barr virus (EBV) oncogenic virus and the second cancer condition is associated with an EBV-free status. The plurality of nucleic acid probes includes at least five nucleic acid probes, and each of the at least five nucleic acid probes includes a respective nucleic acid sequence that is identical or complementary to at least 10 consecutive bases of an RNA transcript of a different respective gene selected from the genes listed in Table 22.

In some embodiments, the plurality of nucleic acid probes includes at least ten probes with sequences that are complementary to or identical to sequences from different genes listed in Table 22. In some embodiments, the plurality of nucleic acid probes includes 2, 3, 4, 5, 6, 7, 8, or 9 probes with sequences that are complementary to or identical to sequences from different genes listed in Table 22.

In some embodiments, the plurality of nucleic acid probes includes one or more probes that bind to a sequence of a gene that is not listed in Table 22. In some embodiments, the plurality of nucleic acid probes includes at least 2, 3, 4, 5, 6, 7, 8, 9, 10, or more probes that bind to a sequence of a gene that is not listed in Table 22. In some embodiments, the plurality of nucleic acid probes includes probes with sequences that bind to no more than 20 genes. In some embodiments, the plurality of nucleic acid probes includes probes with sequences that bind to no more than 25 genes. In some embodiments, the plurality of nucleic acid probes includes probes with sequences that bind to no more than 50 genes. In some embodiments, the plurality of nucleic acid probes includes probes with sequences that bind to no more than 10, 15, 20, 25, 30, 35, 40, 50, 60, 70, 80, 90, 100, 125, 150, 175, 200, 250, or 300 genes.

In some embodiments, each probe in the plurality of probes includes a nucleic acid sequence that is identical or complementary to at least 15 consecutive bases of an RNA transcript of interest, e.g., a transcript from a gene listed in Table 22. In some embodiments, each probe in the plurality of probes includes a nucleic acid sequence that is identical or complementary to at least 30 consecutive bases of an RNA transcript of interest, e.g., a transcript from a gene listed in Table 22. In some embodiments, each probe in the plurality of probes includes a nucleic acid sequence that is identical or complementary to at least 50 consecutive bases of an RNA transcript of interest, e.g., a transcript from a gene listed in Table 22. In some embodiments, each probe in the plurality of probes includes a nucleic acid sequence that is identical or complementary to at least 10, 15, 20, 25, 30, 35, 40, 50, 60, 70, 80, 90, 100, 125, 150, 175, 200, or more consecutive bases of an RNA transcript of interest, e.g., a transcript from a gene listed in Table 22.

### RNA Analysis Pipeline

In some embodiments, the methods and systems described herein are performed in conjunction with sequencing of RNA molecules isolated from a biological sample of a patient. In some embodiments, a FASTQ file, or equivalent file format, of the sequencing data is the output of such a sequencing reaction.

In some embodiments, each FASTQ file contains reads that may be paired-end or single reads, and may be short-reads or long-reads, where each read shows one detected sequence of nucleotides in an mRNA molecule that was isolated from the patient sample, inferred by using the sequencer to detect the sequence of nucleotides contained in a cDNA molecule generated from the isolated mRNA molecules during library preparation. Each read in the FASTQ file is also associated with a quality rating. The quality rating may reflect the likelihood that an error occurred during the sequencing procedure that affected the associated read.

Each FASTQ file may be processed by a bioinformatics pipeline. In various embodiments, the bioinformatics pipeline may filter FASTQ data. Filtering FASTQ data may include correcting sequencer errors and removing (trimming) low quality sequences or bases, adapter sequences, contaminations, chimeric reads, overrepresented sequences, biases caused by library preparation, amplification, or capture, and other errors. Entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools. FASTQ files may be analyzed for rapid assessment of quality control and reads, for example, by a sequencing data QC software such as AfterQC, Kraken, RNA-SeQC, FastQC, (see Illumina, BaseSpace Labs or https://www.illumina.com/products/by-type/informatics-products/basespace-sequence-hub/apps/fastqc.html), or another similar software program. For paired-end reads, reads may be merged.

For each FASTQ file, each read in the file may be aligned to the location in the reference genome having a sequence that best matches the sequence of nucleotides in the read. There are many software programs designed to align reads, for example, Bowtie, Burrows Wheeler Aligner (BWA), programs that use a Smith-Waterman algorithm, etc. Alignment may be directed using a reference genome (for example, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. The alignment may take RNA splice sites into account. The alignment may generate a SAM file, which stores the locations of the start and end of each read in the reference genome and the coverage (number of reads) for each nucleotide in the reference genome. The SAM files may be converted to BAM files, BAM files may be sorted, and duplicate reads may be marked for deletion.

In one example, kallisto software may be used for alignment and RNA read quantification (see Nicolas L Bray, Harold Pimentel, Páll Melsted and Lior Pachter, Near-optimal probabilistic RNA-seq quantification, Nature Biotechnology 34, 525-527 (2016), doi:10.1038/nbt.3519). In an alternative embodiment, RNA read quantification may be conducted using another software, for example, Sailfish or Salmon (see Rob Patro, Stephen M. Mount, and Carl Kingsford (2014) Sailfish enables alignment-free isoform quantification from RNA-seq reads using lightweight algorithms. Nature Biotechnology (doi:10.1038/nbt.2862) or Patro, R., Duggal, G., Love, M. I., Irizarry, R. A., & Kingsford, C. (2017). Salmon provides fast and bias-aware quantification of transcript expression. Nature Methods.). These RNA-seq quantification methods may not require alignment. There are many software packages that may be used for normalization, quantitative analysis, and differential expression analysis of RNA-seq data.

For each gene, the raw RNA read count for a given gene may be calculated. The raw read counts may be saved in a tabular file for each sample, where columns represent genes and each entry represents the raw RNA read count for that gene. In one example, kallisto alignment software calculates raw RNA read counts as a sum of the probability, for each read, that the read aligns to the gene. Raw counts are therefore not integers in this example.

Raw RNA read counts may then be normalized to correct for GC content and gene length, for example, using full quantile normalization and adjusted for sequencing depth, for example, using the size factor method. In one example, RNA read count normalization is conducted according to the methods disclosed in U.S. Pat. App. No. 16/581,706 or PCT19/52801, titled Methods of Normalizing and Correcting RNA Expression Data and filed Sep. 24, 2019, which are incorporated by reference herein in their entirety. The rationale for normalization is the number of copies of each cDNA molecule in the sequencer may not reflect the distribution of mRNA molecules in the patient sample. For example, during library preparation, amplification, and capture steps, certain portions of mRNA molecules may be over or under-represented due to artifacts that arise during various aspects of priming of reverse transcription caused by random hexamers, amplification (PCR enrichment), rRNA depletion, and probe binding and errors produced during sequencing that may be due to the GC content, read length, gene length, and other characteristics of sequences in each nucleic acid molecule. Each raw RNA read count for each gene may be adjusted to eliminate or reduce over- or under-representation caused by any biases or artifacts of NGS sequencing protocols. Normalized RNA read counts may be saved in a tabular file for each sample, where columns represent genes and each entry represents the normalized RNA read count for that gene.

A transcriptome value set may refer to either normalized RNA read counts or raw RNA read counts, as described above.

### Generating a Clinical Report and Assigning Therapy

In some embodiments, the results of the classification described above, e.g., of whether or not the subject is afflicted with a particular oncogenic pathogen, are used to further classify a cancer status of the subject. For instance, in some embodiments, additional types of information derived from the same biological sample, a different biological sample for the individual, and/or a personal survey of the subject, are combined with the classification results to provide diagnosis, prognosis, or treatment recommendations for the subject. These additional types of information can include one or more of genomic information (e.g., sequencing information such as germline or cancer variant allele identification, copy number variation, chromosomal aberration data, etc.), exome information (e.g., gene expression data), epigenetic information (e.g., methylation data, and histone modification data), proteomic information (e.g., protein expression data), metabolome information (e.g., data on the metabolism of the subject), and personal characteristics (e.g., age, weight, smoking status, familial disease history, etc.). For instance, as shown in FIG. 2, different portions of the biological sample, or different biological samples, may be analyzed at different diagnostic environments, e.g., a clinical environment 220, a sequencing lab 230, a pathology lab 240, or a molecular biology lab 250, and the information analyzed at a remove processing/storage center 260.

Methods for classifying the cancer status of an individual are known in the art. For instance, U.S. Provisional Application Serial No. 62/855,750, filed May 31, 2019, and incorporated by reference herein, describes various methods for combining different types of data about a subject in order to classify the cancer status of the subject. In some embodiments, the methods for detecting the presence of an oncogenic pathogen described herein are combined with any of the methods for classifying the cancer status of a subject, as described in USSN 62/855,750.

In some embodiments, the methods for detecting the presence of an oncogenic pathogen described herein are integrated (5150) with a test to determine whether the subject has a type of cancer. In some embodiments, the test determines whether the subject has a type of cancer selected from one or more of breast cancer, lung cancer, prostate cancer, colorectal cancer, renal cancer, uterine cancer, pancreatic cancer, esophagus cancer, head/neck cancer, ovarian cancer, hepatobiliary cancer, cervical cancer, thyroid cancer, or bladder cancer. In some embodiments, the test determines a likelihood that the subject has a particular type of cancer, e.g., a likelihood that the subject has breast cancer, lung cancer, prostate cancer, colorectal cancer, renal cancer, uterine cancer, pancreatic cancer, esophagus cancer, head/neck cancer, ovarian cancer, hepatobiliary cancer, cervical cancer, thyroid cancer, or bladder cancer.

In some embodiments, the methods for detecting the presence of an oncogenic pathogen described herein are integrated with a test to classify a stage of a cancer in the subject, e.g., whether the subject’s cancer is stage I, stage II, stage III, or stage IV cancer. In some embodiments, the test determines the stage of a breast cancer, lung cancer, prostate cancer, colorectal cancer, renal cancer, uterine cancer, pancreatic cancer, esophagus cancer, head/neck cancer, ovarian cancer, hepatobiliary cancer, cervical cancer, thyroid cancer, or bladder cancer.

In some embodiments, the methods for detecting the presence of an oncogenic pathogen described herein are integrated with a test to classify a prognosis for a cancer in a subject, e.g., a survival rate without treatment, a survival rate with treatment, a disease-free survival rate, a cancer recursion rate, etc. In some embodiments, the prognosis is a 1-year, 2-year, 3-year, 4-year, 5-year, or 10-year prognosis, e.g., a ten year disease-free survival rate.

In some embodiments, the methods for detecting the presence of an oncogenic pathogen described herein are integrated with a test to determine a recommended treatment for a cancer in a subject. In some embodiments, the recommended treatment is dependent upon whether or not the subject is afflicted with a particular oncogenic pathogen. Examples of such conditional therapies are provided below in conjunction with FIGS. 3 and 5. For example, non-limited examples of ongoing clinical trials of therapies for particular cancer types that are associated with oncogenic pathogen infections are provided in Table 3, below.

In some embodiments, when the subject is determined to have a first cancer condition, associated with an oncogenic pathogen infection, the method includes assigning and/or administering immunotherapy to the subject. In some embodiments, when the subject is determined to have a second cancer condition, that is not associated with an oncogenic pathogen infection, the method includes assigning and/or administering chemotherapy to the subject.

As summarized in Table 3, several clinical trials are ongoing for the treatment of virally associated tumors. Accordingly, in some embodiments, the methods described herein include assigning and/or administering a treatment for a particular cancer associated with a particular oncogenic viral infection, as listed in Table 3. For example, in some embodiments, upon a determination that the subject has a phase 3 cervical cancer associated with an HPV infection, the subject is assigned and/or administered a therapeutically effective dosing regimen of axalimogene filolisbac, which is a live attenuated Listeria monocytogenes transfected with plasmids encoding the HPV-16E7 protein fused to a truncated fragment of the Lm protein listeriolysin O.

TABLE 3

Clinical trials for the treatment of cancers associated with oncogenic viral infections

| | | | |
|---|---|---|---|
| Therapy | Mechanism of Action | Virus | Cancer / Stage of Development / Clinical Trial |
| Axalimogene filolisbac (AXAL/ADXS 11-001) | Therapeutic vaccine | HPV | Phase 3 cervical cancer (AIM2CERV; NCT02853604); Phase 2 NSCLC (NCT02531854); Phase ½ HNSCC (NCT02291055) |
| TG4001 | Therapeutic vaccine | HPV | Phase ½ HNSCC (NCT03260023) |
| GX-188E | Therapeutic vaccine | HPV | Phase ½ cervical cancer (NCT03444376) |
| VGX-3100 | Therapeutic vaccine | HPV | Phase 3 cervical cancer (REVEAL; NCT03185013); Phase 2 vulval cancer (NCT0318-684) |
| MEDI-0457 | Therapeutic vaccine | HPV | Phase 2 HPV+ cancer (NCT03439085); Phase ½ HNSCC (NCT03162224) |
| INO-3106 | Therapeutic vaccine | HPV | Phase 1 HPV+ cancers (NCT02241369) |
| TA-CIN | Therapeutic vaccine | HPV | Phase 1 cervical cancer (NCT02405221) |
| TA-HPV | Therapeutic vaccine | HPV | Phase 1 cervical cancer (NCT00788164) |
| ISA-101 | Therapeutic vaccine | HPV | Phase 2 HNSCC (NCT03258008) |
| PepCan | Therapeutic vaccine | HPV | Phase 2 cervical cancer (NCT02481414) |
| Nivolumab (Opdivo) | Immune checkpoint inhibitor | HPV | Phase 2 HNSCC (NCT03342911) |
| AMG319 | PI3K inhibitor | HPV | Phase 2 HNSCC (NCT02540928) |
| BKM120 | PI3K inhibitor | HPV | Phase 1 HNSCC (NCT02113878) |
| HPV-specific T cells | Adoptive cell therapy | HPV | Phase 1 HPV+ tumors (NCT02379520); Phase 1 vulvar cancers (NCT03197025) |
| ATA 129 | Adoptive cell therapy | EBV | Phase 3 EBV+ lymphoproliferative disease (NCT03394365/ALLELE, NCT03392142/MATCH) |
| EBVST | Adoptive cell therapy | EBV | Phase 3 EBV+ nasopharyngeal carcinoma (NCT02578641) |
| CMD-003 | Adoptive cell therapy | EBV | Phase 2 EBV+ lymphomas (NCT02763254, NCT01948180/CITADEL) |
| Ibrutinib | BTK inhibitor | EBV | Phase 2 EBV+ DLBCL (NCT02670616) |
| Pembrozilumab | Immune checkpoint inhibitor | EBV | Phase 2 EBV+ gastric cancer (NCT03257163); Phase 1 KSHV+ Kaposo sarcoma (NCT02595866) |
| Nivolumab | Immune checkpoint inhibitor | EBV | Phase 2 EBV+ lymphoproliferative disorders and NHL (NCT03258567) |
| Avelumab | Immune checkpoint inhibitor | MCV | Phase ½ MCV+ MCC (NCT02584829) |
| Talimogene laherparepvec | Vaccine | MCV | Phase 2 MCV+ MCC (NCT02819843) |
| Sapanisertib | mTOR inhibitor | MCV | Phase ½ MCV+ MCC (NCT02514824) |

Similarly, in one embodiment, a method is provided for treating cervical cancer in a human cancer patient. The method includes determining whether the human cancer patient is infected with a human papillomavirus (HPV) oncogenic virus by using a sequence read computational subtraction processes described herein. The method then includes assigning or administering treatment for the cervical cancer, based on whether or not the subject is afflicted with an HPV oncogenic virus. When it is determined that the human cancer patient is infected with an HPV oncogenic virus, a first therapy is assigned or administered that is tailored for treatment of cervical cancer associated with an HPV infection. When it is determined that the human cancer patient is not infected with an HPV oncogenic virus, a second therapy is assigned or administered that is tailored for treatment of cervical cancer not associated with an HPV infection.

In some embodiments, the first therapy tailored for treatment of cervical cancer associated with an HPV infection is a therapeutic vaccine. In some embodiments, the therapeutic vaccine is selected from axalimogene filolisbac (Advaxis), TG4001 (Transgene), GX-188E (Genexine), VGX-3100 (Inovio), MEDI-0457 (Inovio), INO-3106 (Inovio), TA-CIN (Cancer Research Technology), TA-HPV (Cancer Research Technology), ISA-101 (Isa), and PepCan (University of Arkansas).

In some embodiments, the first therapy tailored for treatment of cervical cancer associated with an HPV infection is an adoptive cell therapy. In some embodiments, adoptive cell therapy includes the administration of HPV-specific T cells, for example, as described for clinical trial ID NCT02379520 or NCT03197025 (Baylor College of Medicine).

In some embodiments, the first therapy tailored for treatment of cervical cancer associated with an HPV infection is an immune checkpoint inhibitor. In some embodiments, the immune checkpoint inhibitor is nivolumab (Bristol-Myers Squibb).

In some embodiments, the first therapy tailored for treatment of cervical cancer associated with an HPV infection is a PI3K inhibitor. In some embodiments, the PI3K inhibitor is AMG319 (Amgen) or BKM120 (Novartis).

Similarly, in one embodiment, a method is provided for treating head and neck cancer in a human cancer patient. The method includes determining whether the human cancer patient is infected with a human papillomavirus (HPV) oncogenic virus by using a sequence read computational subtraction processes described herein. The method then includes assigning or administering treatment for the head and neck cancer, based on whether or not the subject is afflicted with an HPV oncogenic virus. When it is determined that the human cancer patient is infected with an HPV oncogenic virus, a first therapy is assigned or administered that is tailored for treatment of head and neck cancer associated with an HPV infection. When it is determined that the human cancer patient is not infected with an HPV oncogenic virus, a second therapy is assigned or administered that is tailored for treatment of head and neck cancer not associated with an HPV infection.

In some embodiments, the first therapy tailored for treatment of head and neck cancer associated with an HPV infection is a therapeutic vaccine. In some embodiments, the therapeutic vaccine is selected from axalimogene filolisbac (Advaxis), TG4001 (Transgene), GX-188E (Genexine), VGX-3100 (Inovio), MEDI-0457 (Inovio), INO-3106 (Inovio), TA-CIN (Cancer Research Technology), TA-HPV (Cancer Research Technology), ISA-101 (Isa), and PepCan (University of Arkansas).

In some embodiments, the first therapy tailored for treatment of head and neck cancer associated with an HPV infection is an adoptive cell therapy. In some embodiments, adoptive cell therapy includes the administration of HPV-specific T cells, for example, as described for clinical trial ID NCT02379520 or NCT03197025 (Baylor College of Medicine).

In some embodiments, the first therapy tailored for treatment of head and neck cancer associated with an HPV infection is an immune checkpoint inhibitor. In some embodiments, the immune checkpoint inhibitor is nivolumab (Bristol-Myers Squibb).

In some embodiments, the first therapy tailored for treatment of head and neck cancer associated with an HPV infection is a PI3K inhibitor. In some embodiments, the PI3K inhibitor is AMG319 (Amgen) or BKM120 (Novartis).

In some embodiments, the method further includes assigning therapy and/or administering therapy to the subject based on the classification of the cancer condition, e.g., based on whether or not the subject’s cancer is associated with an EBV viral infection.

Accordingly, in one embodiment, a method is provided for treating gastric cancer in a human cancer patient. The method includes determining whether the human cancer patient is infected with a Epstein-Barr virus (EBV) oncogenic virus by using a sequence read computational subtraction processes described herein. The method then includes assigning or administering treatment for the gastric cancer, based on whether or not the subject is afflicted with an EBV oncogenic virus. When it is determined that the human cancer patient is infected with an EBV oncogenic virus, a first therapy is assigned or administered that is tailored for treatment of gastric cancer associated with an EBV infection. When it is determined that the human cancer patient is not infected with an EBV oncogenic virus, a second therapy is assigned or administered that is tailored for treatment of gastric cancer not associated with an EBV infection.

In some embodiments, the first therapy tailored for treatment of gastric cancer associated with an EBV infection is an adoptive cell therapy. In some embodiments, the adoptive cell therapy includes is ATA 129 (Atara), EBVST (Tessa), or CMD-003 (Cell Medica).

In some embodiments, the first therapy tailored for treatment of gastric cancer associated with an EBV infection is an immune checkpoint inhibitor. In some embodiments, the immune checkpoint inhibitor is Pembrozilumab (Merck) or nivolumab (Bristol-Myers Squibb).

In some embodiments, the first therapy tailored for treatment of gastric cancer associated with an EBV infection is a BTK inhibitor. In some embodiments, the BTK inhibitor is ibrutinib (Pharmacyclics).

In some embodiments, the method further includes assigning therapy and/or administering therapy to the subject based on the classification of the cancer condition, e.g., based on whether or not the subject’s cancer is associated with a Merkel cell polyomavirus (MCPyV) infection.

Accordingly, in one embodiment, a method is provided for treating a carcinoma in a human cancer patient. The method includes determining whether the human cancer patient is infected with a Merkel cell polyomavirus (MCPyV) oncogenic virus by using a sequence read computational subtraction processes described herein. The method then includes assigning or administering treatment for the carcinoma, based on whether or not the subject is afflicted with a MCPyV oncogenic virus. When it is determined that the human cancer patient is infected with a MCPyV oncogenic virus, a first therapy is assigned or administered that is tailored for treatment of Merkel cell carcinoma associated with a MCPyV infection. When it is determined that the human cancer patient is not infected with a MCPyV oncogenic virus, a second therapy is assigned or administered that is tailored for treatment of carcinoma not associated with a MCPyV infection.

In some embodiments, the treatment tailored to Merkel cell carcinoma is determined based on the stage of the Merkel cell carcinoma. For instance, the National Cancer Institute recommends treating stage I or stage II Merkel cell carcinoma by surgery to remove the tumor, with or without lymph node dissection, and radiation therapy after surgery. In contrast, the National Cancer Institute recommends treating stage III Merkel cell carcinoma by one or more of wide local excision with or without lymph node dissection, radiation therapy, immunotherapy for tumors that cannot be removed by surgery, e.g., immune checkpoint inhibitor therapy using pembrolizumab, a chemotherapy being evaluated in a clinical trial for Merkel cell carcinoma, and an immunotherapy being evaluated in a clinical trial for Merkel cell carcinoma, e.g., nivolumab. Similarly, the National Cancer Institute recommends treating stage IV Merkel cell carcinoma by one or more of immunotherapy, e.g., immune checkpoint inhibitor therapy using pembrolizumab or avelumab, chemotherapy, surgery or radiation therapy as palliative treatment to relieve symptoms and improve quality of life, and an immunotherapy being evaluated in a clinical trial for Merkel cell carcinoma, e.g., nivolumab and ipilimumab. Accordingly, in some embodiments, particularly when the cancer is classified as stage III or stage IV cancer, when it is determined that the human cancer patient is afflicted with a MCPyV oncogenic virus, the patient is assigned or administered immune checkpoint inhibitor therapy, for example an anti-PD1 (e.g., nivolumab, pembrolizumab, or cemiplimab), and anti-PD-L1 (e.g., atezolizumab, avelumab, or duvalumab), or an anti-CTLA-4 (e.g., ipilimumab) monoclonal antibody, and when it is determined that the human cancer patient not is afflicted with a MCPyV oncogenic virus, a therapy is assigned or administered that does not include immune checkpoint inhibitor therapy.

In some embodiments, the methods described herein further include generating (5132) a clinical report for the subject, the clinical report indicating whether the subject is afflicted with an oncogenic pathogen in the plurality of oncogenic pathogens, e.g., using patient reporting module 160.

In some embodiments, the status of the cancer condition is selected from cervical cancer associated with human papilloma virus (HPV), head and neck cancer associated with HPV, gastric cancer associated with Epstein-Barr virus (EBV), nasopharyngeal cancer associated with EBV, Burkitt lymphoma associated with EBV, Hodgkin lymphoma associated with EBV, liver cancer associated with hepatitis B virus (HBV), liver cancer associated with hepatitis C virus (HCV), Kaposi sarcoma associated with Kaposi’s associated sarcoma virus (KSHV), adult T-cell leukemia/lymphoma associated with human T-cell lymphotropic virus (HTLV-1), and Merkel cell carcinoma associated with Merkel cell polyomavirus (MCV). For a summary of cancer conditions known to be associated with an oncogenic pathogen infection, see, for example, de Flora, Carcinogenesis 32:787-95 (2011), which is incorporated herein by reference.

In some embodiments, the subject has cancer, and the clinical report further indicates a type of the cancer, where the indicated type of the cancer is dependent upon whether the subject is afflicted with an oncogenic pathogen in the plurality of oncogenic pathogens (5134). In some embodiments, the type of cancer is selected from breast cancer, lung cancer, prostate cancer, colorectal cancer, renal cancer, uterine cancer, pancreatic cancer, esophagus cancer, head/neck cancer, ovarian cancer, hepatobiliary cancer, cervical cancer, thyroid cancer, or bladder cancer. For example, in one embodiment, when the subject (i) has a B-cell lymphoma and (ii) is afflicted with Epstein-Barr virus, the clinical report indicates that the type of cancer is Epstein-Barr virus-positive mucocutaneous ulcer (EBVMCU) (5136). Similarly, approximately 10-15% of all cases of diffuse large B-cell lymphoma (DLBCL) are associated with the Epstein-Barr virus (EBV). Accordingly, in one embodiment, when the subject (i) has DLBCL and (ii) is afflicted with Epstein-Barr virus, the clinical report indicates that the type of cancer is Epstein-Barr virus-positive DLBCL (EBV + DLBCL).

Other, non-limiting examples of oncogenic pathogens that are known to be associated with specific cancers, such that detection of nucleic acid sequences from these pathogens inform a cancer diagnosis, are shown below in Table 1, above. For additional information on known associations between oncogenic pathogens and cancers see, for example, Flora and Bonanni, 2011, “The prevention of infection-associated cancers,” Carcinogenesis 32(6), pp. 787-795, which is hereby incorporated by reference.

In some embodiments, the subject has metastatic cancer, and the clinical report further indicates a primary origin of the metastatic cancer, where the indicated primary origin of the metastatic cancer is dependent upon whether the subject is afflicted with an oncogenic pathogen in the plurality of oncogenic pathogens (5138). For example, in some embodiments, when the subject (i) has metastatic squamous cell carcinoma (SCC) and (ii) is afflicted with human papillomavirus, the clinical report indicates that the primary origin of the metastatic cancer is the oropharynx (5140). Another example where the association of an oncogenic pathogen with the cancer informs assignment of the primary origin of the cancer is the presence of HPV in any gynecological cancer, which indicates that the primary origin of the cancer is the ovaries. Similarly, the presence of merkel cell polyomavirus in a melanoma indicates that the primary origin of the cancer is a merkel cell.

In some embodiments, the subject has cancer, and the clinical report further indicates a recommended treatment modality for the cancer, where the recommended treatment modality for the cancer is dependent upon whether the subject is afflicted with an oncogenic pathogen in the plurality of oncogenic pathogens (5142). For example, Epstein-Barr virus (EBV) is associated with between 10-15% of all cases of diffuse large B-cell lymphoma (DLBCL). Expression studies of EBV+ and EBV- DLBCL cases show that many genes associated with pathways that are targeted in various cancer therapies (e.g., NF-κB targets, cell cycle regulation genes, anti-apoptosis genes, tumor progression genes, cell proliferation genes, immune response genes, pro-apoptotic genes, etc.) are differentially regulated in EBV+ DLBCL, relative to EBV-DLBCL. Accordingly, it’s been proposed that EBV+ and EBV- DLBCL should be treated differently (see, for example, OK C.Y., et al., Blood, 122(3):328-40, which is incorporated herein by reference). Accordingly, in some embodiments, the subject has lymphoma, and the clinical report indicates: when the subject is determined not to be afflicted with human papillomavirus, that the recommended therapy modality is a chemotherapy or an immunotherapy; and when the subject is determined to be afflicted with human papillomavirus, that the recommended therapy modality is anti-viral therapy (5144). In some embodiments, the subject has lymphoma, and the clinical report indicates: when the subject is determined not to be afflicted with H.pylori, that the recommended therapy modality is a chemotherapy or an immunotherapy; and when the subject is determined to be afflicted with H.pylori, that the recommended therapy modality is antibiotics (5146). In another embodiment, the subject has gastric cancer, and the clinical report indicates that when the subject is afflicted with EBV, the recommended therapy is immunotherapy (e.g., immune checkpoint inhibitor therapy), and when the subject is not afflicted with EBV, the recommended therapy is chemotherapy (e.g., docetaxel, doxorubicin hydrochloride, 5-fluorouracil, fluorouracil, trifluridine and tipiracil hydrochloride, mitomycin C). In yet other embodiments, the recommended treatment modality for a subject afflicted with an oncogenic pathogen is selected from the combination of those diagnoses and treatments shown above in Table 3. Generally, current treatment guidelines for various cancers are maintained by various organizations, including the National Cancer Institute and Merck & Co., in the Merck Manual.

Further, several bacterial species, although not known to contribute to the development of cancer, have been found to confer resistance against specific cancer therapies. For instance, certain bacteria (e.g., Serratia marcescens) express enzymes (e.g., the long isoform of cytidine deaminase) capable of metabolizing gemcitabine into an inactive form. See, for instance, Geller LT et al., Science, 357(6356):1156-60 (2017), which is hereby incorporated by reference. Similarly, certain bacteria (e.g., Bacteroides fragilis) were found to interfere with the efficacy of immune checkpoint inhibitors, such as anti-CTLA-4 monoclonal antibodies. Accordingly, in some embodiments, following identification of a nucleic acid sequence from a bacteria known to confer resistance against a specific cancer therapy, the report generated for the subject indicates that a treatment modality other than the cancer therapy inhibited by the identified bacterium is recommended.

In some embodiments, subject has cancer, and the clinical report further indicates a prognosis for the cancer, where the prognosis for the cancer is dependent upon whether the subject is afflicted with an oncogenic pathogen in the plurality of oncogenic pathogens (5148). For instance, in some embodiments the cancer can be effectively treated by eradicating the underlying oncogenic pathogen infection. In such cases, the prognosis for the cancer patient may be better than for a similar cancer that is not being driven by affliction with an oncogenic pathogen. In contrast, in some embodiments, a cancer associated with an oncogenic pathogen is not as readily treatable as a similar cancer that is not associated with an oncogenic pathogen. In such cases, the prognosis for the cancer patient may be worse than for a cancer patient that is not afflicted with the oncogenic pathogen. Similarly, survival rates for oropharyngeal squamous cell carcinoma (OSCC) associated with HPV are much higher than for OSCC that is not associated with HPV.

### Detection of Non-Oncogenic Pathogens

In some embodiments, in addition to detecting oncogenic pathogens, the systems and methods described herein can also detect non-oncogenic pathogens. For example, in some embodiments, the systems and methods described herein can be used to detect a pathogen that causes an acute disorder, for example, respiratory illnesses (for example, SARS-CoV-1, SARS-CoV-2, MERS-CoV, Coronavirus HKU1, Coronavirus NL63, Coronavirus 229E, Coronavirus OC43, Influenza A, Influenza A H1, Influenza A H1-2009, Influenza A H1N1, Influenza A H3, Influenza B, Influenza C, Parainfluenza virus 1, Parainfluenza virus 2, Parainfluenza virus 3, Parainfluenza virus 4, Rhinovirus/Enterovirus, Adenovirus, Respiratory Syncytial Virus, Respiratory Syncytial Virus A, Respiratory Syncytial Virus B, Human Metapneumovirus, Bocavirus, Human Bocavirus, Chlamydophila pneumoniae, Mycoplasma pneumoniae, Legionella pneumophila, Bordetella, Bordetella holmesii, Bordetella pertussis, Streptococcus pneumoniae, Coxiella burnetii, Staphylococcus aureus, Klebsiella pneumoniae, Moraxella catarrhalis, Haemophilus influenzae, Pneumocystis jirovecii, Enterovirus D68, Epstein-Barr virus (EBV), Mumps, Measles, Cytomegalovirus, Human herpesvirus 6 (HHV-6), Varicella zoster virus (VZV), Parechovirus, etc.), gastroenteritis (for example, norovirus, rotavirus, Escherichia coli/E.coli, Salmonella, Campylobacter, parasites, etc.), meningitis (for example, Steptococcus pneumoniae, Neisseria meningitidis, Haemophilus influenzae type B/Hib), viral hemorrhagic fever (for example, arenaviruses, bunyaviruses, filoviruses, flaviviruses, etc.), cholera (Vibrio cholerae), malaria (including Plasmodium falciparum, P.vivax, P.ovale, P.malariae, P.knowlesi), tuberculosis (including Mycobacterium tuberculosis), measles (including paramyxovirus), pertussis (including Bordetella pertussis), etc.

In some embodiments, the systems and methods described herein can be used to detect a pathogen associated with a chronic disease or other type of disease, for example, hepatitis B virus, hepatitis C virus, human immunodeficiency virus (HIV), pathogens associated with liver disease (including hepatitis A, B, C, D, E virus), Lyme disease, tuberculosis, sexually transmitted diseases, antibiotic resistant bacteria (MRSA, C. difficile), etc. In some embodiments, a method described herein is performed to determine whether a subject is afflicted with an oncogenic pathogen and, at the same time, whether the subject is afflicted with a pathogen that causes an acute disorder or chronic disease. In this fashion, detection of a non-oncogenic pathogen in a sample from a subject with cancer can be reported as an incidental finding. For example, in some embodiments, such a report would alert a physician treating the subject that sequence reads of the pathogen unrelated to the cancer were detected and the patient may need additional testing to confirm the infection. This could catch chronic infections at an early stage, give the patient more treatment options, avoid organ failure and/or compromised immune system in the patient, etc.

Table 27 providing taxonomic identifiers for some of the respiratory pathogens listed above. The taxonomic identifiers can be used to find nucleic acid (genetic) sequences associated with these pathogens in one of several publicly-available databases, such as the NCBI Virus database accessible online at ncbi.nlm.nih.gov/labs/virus/vssi/#/. In various embodiments, the diagnostic test used to detect the presence of a pathogen may detect portions of a genetic sequence associated with the pathogen.

TABLE 27

Example respiratory pathogens

| | |
|---|---|
| Target | Taxonomic Identifiers |
| Adenovirus | taxid: 9605 |
| MERS-CoV | taxid: 1335626 |
| SARS-CoV-1 | taxid: 694009 |
| SARS-CoV-2 | taxid: 2697049 |
| Coronavirus 229E | taxid: 11137 |
| Coronavirus HKU1 | taxid: 290028 |
| Coronavirus NL63 | taxid: 277944 |
| Coronavirus OC43 | taxid: 31631 |
| Human Bocavirus | taxid: 9606 |
| Human Metapneumovirus | taxid: 162145 |
| Influenza A | taxid: 11320 |
| Influenza A/H1 | taxid: 211044 |
| Influenza A/H1-2009 | taxid: 641809 |
| Influenza A/H3 | taxid: 335341 |
| Influenza B | taxid: 518987 |
| Influenza C | taxid: 11552 |
| Parainfluenza virus 1 | taxid: 12730 |
| Parainfluenza virus 2 | taxid: 1979160 |
| Parainfluenza virus 3 | taxid: 11216 |
| Parainfluenza virus 4 | taxid: 11224 |
| Respiratory Syncytial Virus A | taxid: 11247 |
| Respiratory Syncytial Virus B | taxid: 11246 |
| Rhinovirus/Enterovirus* | taxid: 12059 |

### EXAMPLES

### Example 1 - The Cancer Genome Atlas (TCGA)

The Cancer Genome Atlas (TCGA) is a publicly available dataset comprising more than two petabytes of genomic data for over 11,000 cancer patients, including clinical information about the cancer patients, metadata about the samples (e.g. the weight of a sample portion, etc.) collected from such patients, histopathology slide images from sample portions, and molecular information derived from the samples (e.g. mRNA/miRNA expression, protein expression, copy number, etc.). The TCGA dataset includes data on 33 different cancers: breast (breast ductal carcinoma, bread lobular carcinoma) central nervous system (glioblastoma multiforme, lower grade glioma), endocrine (adrenocortical carcinoma, papillary thyroid carcinoma, paraganglioma & pheochromocytoma), gastrointestinal (cholangiocarcinoma, colorectal adenocarcinoma, esophageal cancer, liver hepatocellular carcinoma, pancreatic ductal adenocarcinoma, and stomach cancer), gynecologic (cervical cancer, ovarian serous cystadenocarcinoma, uterine carcinosarcoma, and uterine corpus endometrial carcinoma), head and neck (head and neck squamous cell carcinoma, uveal melanoma), hematologic (acute myeloid leukemia, Thymoma), skin (cutaneous melanoma), soft tissue (sarcoma), thoracic (lung adenocarcinoma, lung squamous cell carcinoma, and mesothelioma), and urologic (chromophobe renal cell carcinoma, clear cell kidney carcinoma, papillary kidney carcinoma, prostate adenocarcinoma, testicular germ cell cancer, and urothelial bladder carcinoma).

### Example 2 - Detection of an Oncogenic Pathogen in a Cervical Cancer Biopsy

In order to test the viral detection method described herein, sequencing data was generated from total nucleic acid isolated from a tumor biopsy of a cervical cancer patient. Briefly, tumor total nucleic acid was extracted from formalin-fixed paraffin-embedded (FFPE) tumor tissue sections that were proteinase K digested. Total nucleic acid was extracted using a source-specific magnetic bead protocol. Total nucleic acid was utilized for all DNA library construction. RNA was purified from the total nucleic acid by DNaseI digestion and magnetic bead purification. Nucleic acids were quantified using commercial DNA or RNA quantification kits.

One hundred nanograms (ng) of isolated DNA was mechanically sheared to an average size of 200 base pairs (bp) using an ultrasonicator. DNA libraries were then prepared using a commercial DNA library preparation kit (e.g., a KAPA Hyper Prep Kit), and hybridized to a targeted probe set (e.g., similar to the probe set shown in FIG. 4A) containing probes against HPV, EBV, and MCV viral sequences. The hybridized nucleic acids were then amplified using a commercial PCR amplification kit (e.g., KAPA HiFi HotStart ReadyMix). One hundred ng of RNA for each tumor sample were fragmented to an average size of 200 bp (e.g., by heat treatment in the presence of magnesium). Library preps were hybridized with a commercial exome panel (e.g., the IDT xGEN Exome Research Panel) and target recovery was performed using Streptavidin-coated beads, followed by amplification with a commercial PCR amplification kit (e.g., KAPA HiFi HotStart ReadyMix). The amplified target-captured DNA tumor libraries were then sequenced to a depth of 65 million total reads by next generation sequencing, resulting in an average sequencing depth across the targets of the probe set of approximately 500x.

The 65 million sequence reads were then aligned to a human reference genome using the Scalable Nucleotide Alignment Program (SNAP) sequence alignment algorithm (Zaharia M., et al., arXiv:1111.5572v1 [cs.DS] 23 Nov. 2011, the content of which is incorporated by reference herein), which was completed in 383 seconds. Parameters and statistics for the alignment, as described in Zaharia et al., are shown in Table 4, below. Of the 65 million sequence reads, 93,781 reads were not aligned to the reference human genome.

TABLE 4

Parameters and statistics for SNAP sequence alignment to a human reference genome

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 12 | 25 | 4 | 99.88% | 0.00% | 99.82% | 0.18% | - |

The 93,781 reads that were not mapped to the human reference genome were then aligned to a comprehensive bacterial genome database (curated by the NCBI) using SNAP. This process took 517 seconds. In contrast, aligning all 65 million of the original sequence reads would have taken nearly 100 hours at the same rate. The 93,781 reads that were not mapped to the human reference genome were also aligned to a comprehensive viral genome database (curated by the NCBI) using SNAP. This process took 152 seconds. In contrast, aligning all 65 million of the original sequence reads would have taken nearly 30 hours at the same rate. Parameters and statistics for the alignment, as described in Zaharia et al., are shown in Tables 5 and 6, below.

TABLE 5

Parameters and statistics for SNAP sequence alignment to a microbial genome database

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 18 | 200 | 4 | 100.00% | 0.00% | 0.60% | 99.40% | - |

TABLE 6

Parameters and statistics for SNAP sequence alignment to a human reference genome

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 18 | 200 | 4 | 100.00% | 0.00% | 0.00% | 100.00% | - |

The species of each aligned bacterial and viral sequence was determined and the number of sequence reads from each species was totaled. The final sequence read counts for each species identified are shown below in Tables 7 and 8.

TABLE 7

Count of microbial sequence reads identified in cervical cancer biopsy

| Sequence Count | Species |
|---|---|
| 1 | Acidovorax_delafieldii |
| 1 | Bacteroides_fragilis |
| 1 | Bradyrhizobium_sp._STM_3809 |
| 112 | Burkholderia_mallei |
| 208 | Candidatus_Pelagibacter_ubique |
| 1 | Corynebacterium_bovis |
| 11 | Cutibacterium_acnes |
| 1 | Escherichia_coli |
| 1 | Gordonia_alkanivorans |
| 1 | Mesorhizobium_alhagi |
| 1 | Mesorhizobium_amorphae |
| 1527 | Microbacterium_laevaniformans |
| 1 | Micrococcus_luteus |
| 47 | Propionibacterium_sp._409-HC1 |
| 24 | Propionibacterium_sp._434-HC2 |
| 2 | Pseudomonas_aeruginosa |
| 1 | Pseudomonas_amygdali |
| 2 | Sphingomonas_sp._KC8 |
| 1 | Sphingomonas_sp._S17 |
| 2 | Staphylococcus_warneri |
| 1 | Verminephrobacter_aporrectodeae |
| 1 | Xanthomonas_citri |

TABLE 8

Count of viral sequence reads identified in cervical cancer biopsy.

| Count | Species |
|---|---|
| 3982 | Alphapapillomavirus_7 |
| 4 | Enterobacteria_phage_phiX174_sensu_lato |
| 1 | Escherichia_virus_alpha3 |
| 1 | Escherichia_virus_phiK |
| 148 | Escherichia_virus_phiX174 |
| 15429 | Human_papillomavirus |

As shown in Table 7, the method identified 15429 Human papillomavirus (HPV) reads, 3982 Alphapapillomavirus 7 reads, and 148 Escherichia virus phiX174 reads, in addition to a low level of three other viruses: Enterobacteria phage phiX174 sensu lato, Escherichia virus alpha3, and Escherichia virus phiK. Because the number of reads for the former, but not the latter, group of viruses satisfied a predetermined threshold of at least 10 sequence reads, the cervical cancer is characterized as afflicted with Human papillomavirus (HPV) and Alphapapillomavirus 7 viral infections. Notably, Human papillomavirus (HPV) and Alphapapillomavirus 7 are known to be associated with human cancers, such that this information could be used to inform treatment of the cervical cancer. The Escherichia virus phiX174 reads can be discounted because the virus is a common contaminant in genome sequencing experiments (see, for example, Mukherjee S., et al., Stand. Genomic Sci. 10:18 (2015)), and does not infect human cells. Notably, this example highlights a case where alignment to only a panel of targeted species of oncogenic pathogen would have missed a less common Alphapapillomavirus 7 viral infection. Particularly, because two strains of papillomavirus were detected in this subject.

### Example 3 – Detection of an Oncogenic Pathogen in a Head and Neck Squamous Carcinoma (HNSCC) Biopsy

In order to test the viral detection method described herein, sequencing data was generated from total nucleic acid isolated from a tumor biopsy of an HNSCC cancer patient. Briefly, tumor total nucleic acid was extracted from formalin-fixed paraffin-embedded (FFPE) tumor tissue sections that were proteinase K digested. Total nucleic acid was extracted using a source-specific magnetic bead protocol. Total nucleic acid was utilized for all DNA library construction. RNA was purified from the total nucleic acid by DNaseI digestion and magnetic bead purification. Nucleic acids were quantified using commercial DNA or RNA quantification kits.

One hundred nanograms (ng) of isolated DNA was mechanically sheared to an average size of 200 base pairs (bp) using an ultrasonicator. DNA libraries were then prepared using a commercial DNA library preparation kit (e.g., a KAPA Hyper Prep Kit), and hybridized to a targeted probe set (e.g., similar to the probe set shown in FIG. 4A) containing probes against HPV, EBV, and MCV viral sequences. The hybridized nucleic acids were then amplified using a commercial PCR amplification kit (e.g., KAPA HiFi HotStart ReadyMix). One hundred ng of RNA for each tumor sample were fragmented to an average size of 200 bp (e.g., by heat treatment in the presence of magnesium). Library preps were hybridized with a commercial exome panel (e.g., the IDT xGEN Exome Research Panel) and target recovery was performed using Streptavidin-coated beads, followed by amplification with a commercial PCR amplification kit (e.g., KAPA HiFi HotStart ReadyMix). The amplified target-captured DNA tumor libraries were then sequenced to a depth of 83 million total reads by next generation sequencing.

The 83 million sequence reads were then aligned to a human reference genome using the Scalable Nucleotide Alignment Program (SNAP) sequence alignment algorithm (Zaharia M., et al., arXiv:1111.5572v1 [cs.DS] 23 Nov. 2011, the content of which is incorporated by reference herein), which was completed in 366 seconds. Parameters and statistics for the alignment, as described in Zaharia et al., are shown in Table 9, below. Of the 83 million sequence reads, 414,645 reads were not aligned to the reference human genome.

TABLE 9

Parameters and statistics for SNAP sequence alignment to a human reference genome.

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 12 | 25 | 4 | 99.92% | 0.00% | 99.08% | 0.92% | - |

The 414,645 reads that were not mapped to the human reference genome were then aligned to a comprehensive bacterial genome database (curated by the NCBI) using SNAP. This process took 464 seconds. In contrast, aligning all 83 million of the original sequence reads would have taken more than 25 hours at the same rate. The 414,645 reads that were not mapped to the human reference genome were also aligned to a comprehensive viral genome database (curated by the NCBI) using SNAP. This process took 195 second. In contrast, aligning all 65 million of the original sequence reads would have taken more than 10 hours at the same rate. Parameters and statistics for the alignments, as described in Zaharia et al., are shown in Tables 10 and 11, below.

TABLE 10

Parameters and statistics for SNAP sequence alignment to a microbial genome database

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 18 | 200 | 4 | 100.00% | 0.00% | 0.00% | 100.00% | - |

TABLE 11

Parameters and statistics for SNAP sequence alignment to a human reference genome

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 18 | 200 | 4 | 100.00% | 0.00% | 0.00% | 100.00% | - |

The species of each aligned bacterial and viral sequence was determined and the number of sequence reads from each species was totaled. The final sequence read counts for each species identified are shown below in Tables 12 and 13.

TABLE 12

Count of microbial sequence reads identified in HNSCC biopsy

| Sequence Count | Species |
|---|---|
| 1 | Acidovorax delafieldii |
| 160 | Burkholderia_mallei |
| 334 | Candidatus Pelaqibacter ubique |
| 1002 | Microbacterium_laevaniformans |
| 1 | Micrococcus_luteus |
| 21 | Propionibacterium_sp._409-HC1 |
| 4 | Propionibacterium_sp._434-HC2 |
| 1 | Vibrio tubiashii |

TABLE 13

Count of viral sequence reads identified in HNSCC biopsy

| Count | Species |
|---|---|
| 1 | Enterobacteria_phage_phiX174_sensu_lato |
| 52 | Escherichia_virus_phiX174 |
| 1 | Feline_leukemia_virus |
| 1469 | Human_gammaherpesvirus_4 |
| 1 | Zantedeschia_mild_mosaic_virus |

As shown in Table 13, the method identified 1469 Human gammaherpesvirus 4 reads and 52 Escherichia virus phiX174 reads, in addition to a low level of three other viruses. Because the number of reads for the former, but not the latter, group of viruses satisfied a predetermined threshold of at least 10 sequence reads, the HNSCC cancer is characterized as afflicted with Human papillomavirus (HPV), Alphapapillomavirus 9. Notably, Human papillomavirus (HPV) and Alphapapillomavirus 9 are known to be associated with human cancers, such that this information could be used to inform treatment of the HNSCC cancer. The Escherichia virus phiX174 reads can be discounted because the virus is a common contaminant in genome sequencing experiments (see, for example, Mukherjee S., et al., Stand. Genomic Sci. 10:18 (2015)), and does not infect human cells.

### Example 4 - Detection of an Oncogenic Pathogen in a Colorectal Cancer Biopsy

In order to test the viral detection method described herein, sequencing data was generated from total nucleic acid isolated from a tumor biopsy of a colorectal cancer patient. Briefly, tumor total nucleic acid was extracted from formalin-fixed paraffin-embedded (FFPE) tumor tissue sections that were proteinase K digested. Total nucleic acid was extracted using a source-specific magnetic bead protocol. Total nucleic acid was utilized for all DNA library construction. RNA was purified from the total nucleic acid by DNaseI digestion and magnetic bead purification. Nucleic acids were quantified using commercial DNA or RNA quantification kits.

One hundred nanograms (ng) of isolated DNA was mechanically sheared to an average size of 200 base pairs (bp) using an ultrasonicator. DNA libraries were then prepared using a commercial DNA library preparation kit (e.g., a KAPA Hyper Prep Kit), and hybridized to a targeted probe set (e.g., similar to the probe set shown in FIG. 4A) containing probes against HPV, EBV, and MCV viral sequences. The hybridized nucleic acids were then amplified using a commercial PCR amplification kit (e.g., KAPA HiFi HotStart ReadyMix). One hundred ng of RNA for each tumor sample were fragmented to an average size of 200 bp (e.g., by heat treatment in the presence of magnesium). Library preps were hybridized with a commercial exome panel (e.g., the IDT xGEN Exome Research Panel) and target recovery was performed using Streptavidin-coated beads, followed by amplification with a commercial PCR amplification kit (e.g., KAPA HiFi HotStart ReadyMix). The amplified target-captured DNA tumor libraries were then sequenced to a depth of 76 million total reads by next generation sequencing.

The 76 million sequence reads were then aligned to a human reference genome using the Scalable Nucleotide Alignment Program (SNAP) sequence alignment algorithm (Zaharia M., et al., arXiv:1111.5572v1 [cs.DS] 23 Nov. 2011, the content of which is incorporated by reference herein), which was completed in 394 seconds. Parameters and statistics for the alignment, as described in Zaharia et al., are shown in Table 14, below. Of the 76 million sequence reads, 92,523 reads were not aligned to the reference human genome.

TABLE 14

Parameters and statistics for SNAP sequence alignment to a human reference genome

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 12 | 25 | 4 | 99.86% | 0.00% | 99.90% | 0.10% | - |

The 92,523 reads that were not mapped to the human reference genome were then aligned to a comprehensive bacterial genome database (curated by the NCBI) using SNAP. This process took 603 seconds. In contrast, aligning all 76 million of the original sequence reads would have taken nearly 140 hours at the same rate. The 92,523 reads that were not mapped to the human reference genome were also aligned to a comprehensive viral genome database (curated by the NCBI) using SNAP. This process took 183 second. In contrast, aligning all 76 million of the original sequence reads would have taken more than 40 hours at the same rate. Parameters and statistics for the alignments, as described in Zaharia et al., are shown in Tables 15 and 16, below.

TABLE 15

Parameters and statistics for SNAP sequence alignment to a microbial genome database

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 18 | 200 | 4 | 100.00% | 0.00% | 0.07% | 99.93% | - |

TABLE 16

Parameters and statistics for SNAP sequence alignment to a human reference genome

| Seed Size | Conf Diff | Max Hits | Max Dist | Max Seed | Conf Ad | % Used | % Unique | % Multi | % !Found | % Error |
|---|---|---|---|---|---|---|---|---|---|---|
| 20 | 2 | 250 | 18 | 200 | 4 | 100.00% | 0.00% | 0.00% | 100.00% | - |

The species of each aligned bacterial and viral sequence was determined and the number of sequence reads from each species was totaled. The final sequence read counts for each species identified are shown below in Tables 17 and 18.

TABLE 17

Count of microbial sequence reads identified in colorectal cancer biopsy

| Sequence Count | Species |
|---|---|
| 1 | Acidovorax_delafieldii |
| 160 | Burkholderia_mallei |
| 334 | Candidatus_Pelaqibacter_ubique |
| 1002 | Microbacterium_laevaniformans |
| 1 | Micrococcus_luteus |
| 21 | Propionibacterium_sp._409-HC1 |
| 4 | Propionibacterium_sp._434-HC2 |
| 1 | Vibrio_tubiashii |

TABLE 18

Count of viral sequence reads identified in colorectal cancer biopsy

| Count | Species |
|---|---|
| 1 | Enterobacteria_phage_phiX174_sensu_lato |
| 52 | Escherichia_virus_phiX174 |
| 1 | Feline_leukemia_virus |
| 1469 | Human_gammaherpesvirus_4 |
| 1 | Zantedeschia_mild_mosaic_virus |

As shown in Table 18, the method identified 1469 Human gammaherpesvirus 4 (also known as Epstein-Barr virus, EBV) reads and 52 Escherichia virus phiX174 reads, in addition to a low level of three other viruses. Because the number of reads for the former, but not the latter, group of viruses satisfied a predetermined threshold of at least 10 sequence reads, the colorectal cancer is characterized as afflicted with EBV. Notably, EBV is associated with at least Hodgkin lymphoma, Burkitt’s lymphoma, and nasopharyngeal cancers. Accordingly, this information could be used to inform treatment of the colorectal cancer. The Escherichia virus phiX174 reads can be discounted because the virus is a common contaminant in genome sequencing experiments (see, for example, Mukherjee S., et al., Stand. Genomic Sci. 10:18 (2015)), and does not infect human cells.

### Example 5 - Detection of an Oncogenic Pathogens in Targeted-Panel Sequencing Data from Assays with and Without Probes Directed to Pathogen Targets

In order to evaluate the improvement in oncogenic pathogen detection provided by using capture probes against one or more viral targets, the bioinformatics method described herein was applied to data generated from molecular biopsy assays the did and did not include such capture probes. As shown in Table 19, inclusion of capture probes against sequences from oncogenic pathogens improved HPV detection by greater than 400% (average detection without oncogenic capture probes = 0.0167; average detection with oncogenic capture probes = 0.686).

TABLE 19

Detection of HPV in data sets generated by various molecular biopsy assays

| Assay | HPV Detected | Total Runs | Percent Positive | Oncogenic Pathogen Probes |
|---|---|---|---|---|
| 1 | 1092 | 60,274 | 0.0181 | No |
| 2 | 118 | 6598 | 0.0179 | No |
| 3 | 1 | 59 | 0.0169 | No |
| 4 | 134 | 9782 | 0.0137 | No |
| 5 | 2220 | 32,236 | 0.0689 | Yes |
| 6 | 687 | 10,061 | 0.0683 | Yes |

Assay 2 sequences the entire coding region (exome) of the human genome. It is optimized for formalin fixed paraffin embedded (FFPE) tumor tissue samples. The FFPE tumor tissue is matched to a normal blood or saliva sample to ensure fidelity of somatic variant calling. Assay 2 is designed to identify actionable oncologic variants as well as neoantigens across the exome thus enabling immuno-oncology applications.

Assay 3 is a non-invasive, liquid biopsy panel of 105 genes focused on oncogenic and resistance mutations in cell-free DNA (cfDNA). The assay provides approximately 20,000x DNA sequencing coverage over the target sequences. This panel is designed to provide clinical decision support for solid tumors.

Assay 4 combines a 595 gene somatic and germline DNA sequencing panel with RNA-sequencing. For solid tumors, it uses an FFPE tumor sample with a matched normal saliva or blood sample. For circulating hematologic malignancies, a blood or bone marrow sample is used. The assay is designed to identify actionable oncologic variants and is capable of detecting both somatic and germline single nucleotide polymorphisms (SNPs), indels less than 100 bp, copy number variants, and rearrangements in a targeted subset of clinically actionable genes via a single DNA sample. Further information on Assay 4 is provided in Beaubier N, et al., Oncotarget, 10(24):2384-96 (2019), which is incorporated by reference herein. Assays 5 and 6 integrate target probes against the oncogenic pathogen genes listed in Table 2 into the framework of Assay 4.

### Example 6 – RNA Expression Profiling

Referring to FIG. 8, the expression profile of genes useful for determining HPV viral status was determined from a tumor sample of a head and neck cancer.

In accordance with block 1302 of FIG. 8, a tumor biopsy of a head and neck cancer was obtained from a cancer patient, using a biopsy technique as described herein. The biopsy was flash frozen in liquid nitrogen shortly after removal from the patient.

In accordance with block 1304 of FIG. 8, mRNA was isolated from the tumor sample. Briefly, the sample tissue block was removed from the liquid nitrogen, and a 5 mm × 5 mm × 5 mm block of the sample was removed and dissected using a cold knife. The dissected sample was mixed with TRIzol reagent (Chomczynski and Sacchi, 1987, Anal Biochem. 162(1), pp. 156-59, the content of which is incorporated herein by reference in its entirety, for all purposes) and homogenized by three short cycles, e.g., 60 seconds, 30 seconds, and 30 seconds, using a tissue homogenizer. Chloroform was added to the homogenized tumor sample, and the reaction was mixed. After phase separation, the aqueous phase of the reaction was removed and mixed with equal parts isopropanol, to precipitate the RNA. The reaction was centrifuged to pellet the RNA, the supernatant was removed. The pellet was washed twice with cold ethanol and then air dried. The extracted RNA was then re-suspended in RNase-free water.

Referring to block 1306 of FIG. 8, mRNA in the isolated RNA was then quantified by whole exome sequencing. In accordance with block 1308 of FIG. 8, mRNA was isolated from the extracted RNA by annealing to magnetic oligo(dT)-conjugated beads by heating the extracted RNA to disrupt secondary structures, and then incubating the RNA with the oligo(dT)-conjugated beads with the denatured RNA at room temperature in hybridization buffer. The beads were recovered and washed twice with hybridization buffer. The hybridized mRNA was then eluted by heating and recovered from the reaction.

In accordance with block 1310 of FIG. 8, a cDNA library was constructed from the isolated mRNA. Briefly, divalent cations were added to the isolated mRNA to fragment the molecules at high temperature. The fragmented mRNA was precipitated by incubating at -80° C. in ethanol at pH 5.2, using glycogen as a carrier molecule. The mRNA was pelleted by centrifugation, washed with 70% ethanol, air dried, then re-suspended in RNase-free water. First strand DNA synthesis was performed using random primers and a reverse transcriptase enzyme. Second strand DNA synthesis was then performed using a DNA polymerase in the presence of RNaseH, to form double stranded cDNA. 5′-overhangs created by the second strand synthesis were repaired using T4 and Klenow DNA polymerases, to form blunt ends. The 3′-ends of the blunt-end cDNA were adenylated using Klenow DNA polymerase. Adapters were ligated to the ends of the adenylated cDNA using T4 DNA ligase, and the cDNA templates were purified and sized by agarose electrophoresis. Optionally, the purified cDNA templates are enriched by PCR amplification, thereby forming the final cDNA library.

In accordance with block 1312 of FIG. 8, whole exome sequencing of the cDNA library was performed using the integrated DNA technologies (IDT) XGEN® LOCKDOWN® technology with the xGen Exome Research Panel. Briefly, the xGen Exome Research Panel covers 51 Mb of end-to-end tiled probe space of the human genome, providing deep and uniform coverage for whole exome target capture. The cDNA library was hybridized to biotinylated-DNA capture probes covering a reference human exome. The hybridized probes were recovered by binding to streptavidin beads. Post-capture PCR was performed to enrich the captured sequences. The amplified products were then sequenced using sequencing by synthesis (SBS) technology (Bently et al., 2008, Nature 456(7218), pp. 53-59, the content of which is hereby incorporated herein by reference, in its entirety, for all purposes).

The RNA sequencing data was then normalized using gene length data, guanine-cytosine (GC) content data, and depth of sequencing data, by normalizing the gene length data for at least one gene to reduce systematic bias, normalizing the GC content data for the at least one gene to reduce systematic bias, and normalizing the depth of sequencing data for each sample, as described in U.S. Provisional Application Serial No. 62/735,349 and U.S. Pat. Application Serial No. 16/581,706, the contents of which are hereby incorporated herein by reference, in their entireties, for all purposes. The RNA sequencing data was also corrected against a standard gene expression dataset by comparing the sequence data for at least one gene in the gene expression dataset to sequence data in the standard gene expression dataset, as described in U.S. Provisional Application Serial No. 62/735,349 and U.S. Pat. Application Serial No. 16/581,706. The normalized and corrected RNA expression data for the twenty-four genes identified in Table 21, as well as the patient’s CDKN2A and TP53 allele statuses, were then input into the HPV detection classifier trained in Example 3, to determine the HPV viral status of the patient.

### Example 7 – Human Papilloma Virus Detection

Referring to FIGS. 9A through 9D, a classifier for determining HPV viral status was trained using gene expression from the tumor RNA-seq data of a training population, where each subject in the training population had been diagnosed with head and neck squamous cell carcinoma or with cervical cancer.

In accordance with block 1204 of FIG. 7A, a training dataset was obtained. Here, the dataset comprised a corresponding plurality of abundance values for each subject in the TCGA, described in Example 1, that had cervical cancer or head and neck cancer with known HPV status. As illustrated in FIG. 9A, there were 427 subjects in the TCGA that satisfied these selection criteria and thus served as the plurality of subjects of the training dataset. Of the 427 subjects, 263 had head and neck cancer and 164 has cervical cancer. Of the 263 subjects that had head and neck cancer, 32 tested positive for HPV and 231 tested negative for HPV. Of the 164 subjects that had cervical cancer, 156 tested positive for HPV and 8 tested negative for HPV. Thus, of the 427 subjects, 188 subjects were deemed to have the first cancer condition (afflicted with HPV and having head and neck, or cervical cancer) and the remaining 239 subjects were deemed to have the second cancer condition (not afflicted with HPV, but having head and neck, or cervical cancer).

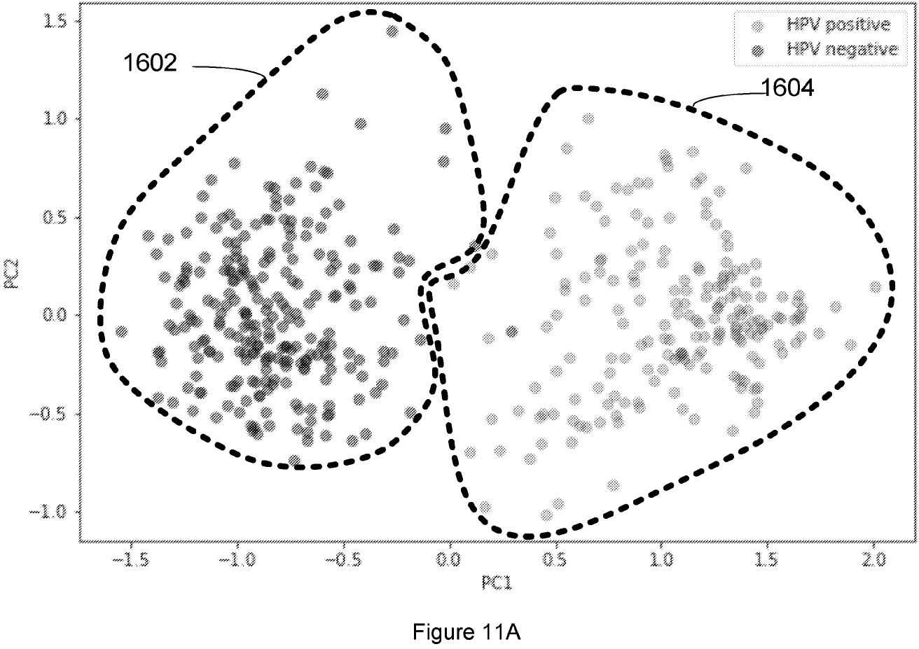

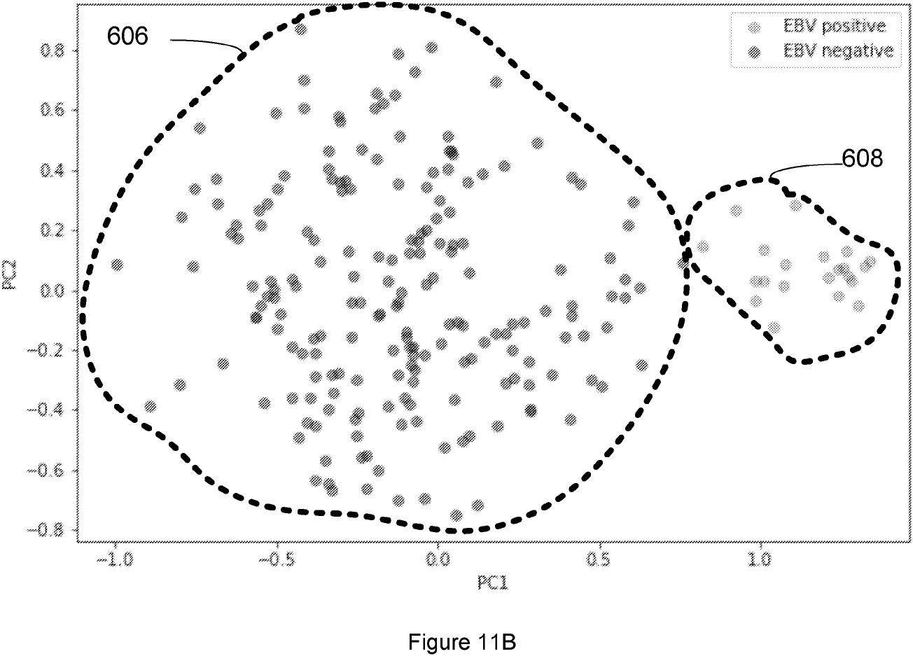

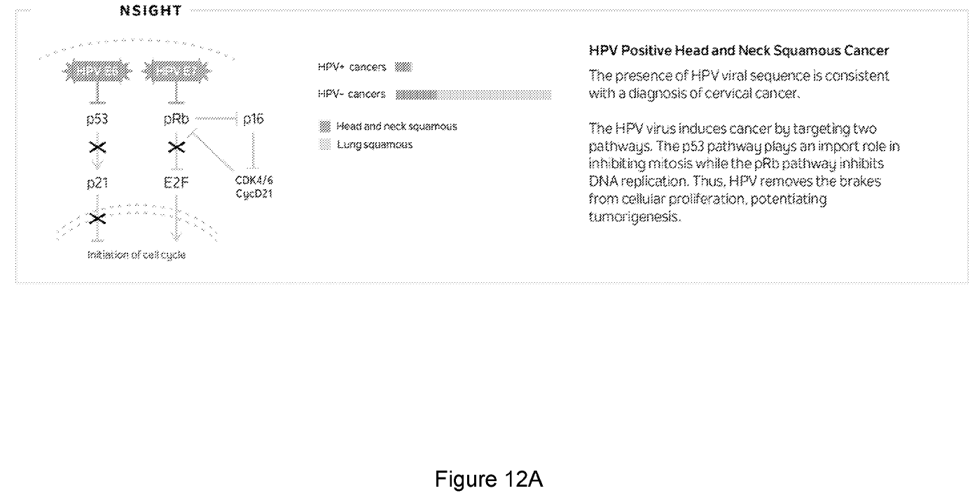

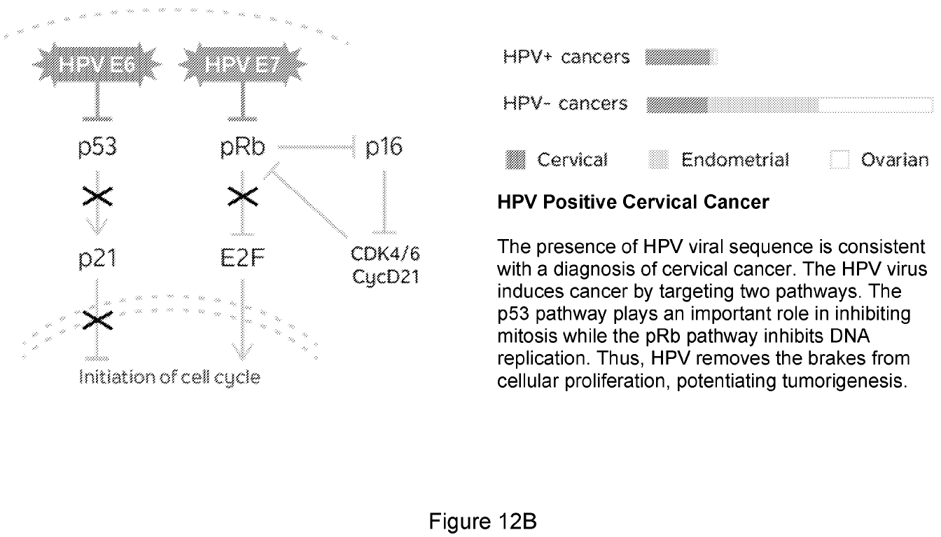

Next, in accordance with block 1218 of FIG. 7C and block 1228 of FIG. 7D, the gene expression values from whole exome RNA data in the TCGA dataset for the 427 subjects was used to identify a discriminating gene set by regression, in which the gene expression values obtained from whole exome mRNA expression data for the 427 subjects in the TCGA dataset served as independent variables and the indication of whether a respective subject had the first cancer condition (afflicted with HPV and having head and neck, or cervical cancer) or the second cancer condition (not afflicted with HPV, but having head and neck, or cervical cancer) served as the dependent variable. More specifically, in accordance with block 1228 of FIG. 7D, the dataset consisting of 427 subjects was split into ten sets (ten splits). Each set included two or more subjects afflicted with the first cancer condition and two or more subjects afflicted with the second cancer condition. Each respective set of the ten sets (splits) was independently subjected to regression in which whole exome mRNA expression data for the subjects of the respective set served as independent variables and the indication of whether a respective subject in the respective set had the first or second cancer condition served as the dependent variable. Each regression (split) was performed with L1 (LASSO) regularization in accordance with block 1238 of FIG. 2E. Since L1 regularization leads to sparse coefficients, only a small subset of genes had non-zero coefficients for each set. Only the genes with non-zero coefficients in more than 80% of the sets were included in the final model. In other words, only those genes that had non-zero regression coefficients for at least eight of the ten sets (splits) were accepted into the discriminating set of genes on the basis of their expression data. The list of genes that satisfied this requirement are the ones listed in FIG. 9B in which the feature type is “gene expression.” Furthermore, FIG. 11A illustrates principal component analysis of the abundance values of the genes listed in FIG. 9B across the training set. FIG. 11A illustrates that a plot of the first and second PCA values for each of the subjects in the training set break out into two distinct groups, corresponding to the first cancer condition (group 1602) and second cancer condition (1604), indicating the power of the abundance values of the genes listed in FIG. 9B to discriminate between the first and second cancer state.

In some embodiments, additional genes were included in the discriminating set of genes based on the presence or absence of mutations (e.g., the number of mutations) in the additional genes. In this example, as detailed in FIG. 9B, the genes CDKN2A and TP53 were included in the discriminating set of genes and the feature for these genes was the number of times mutations were observed in these genes in each of the respective 427 subjects of the training set.

Next, in accordance with block 1242 of FIG. 7E, the respective abundance values for the discriminating gene set and the respective indication of cancer condition across the 427 subjects was used to train a classifier to discriminate between the first and second cancer conditions as a function of respective abundance values for the discriminating gene set. In a first model, the classifier used was a logistic regression classifier with a L1 regularization, in which the training was the 427 subjects but only using TCGA gene abundance levels for the genes listed in FIG. 9B for which the feature is “gene expression.” In a second model, the classifier used was a logistic regression classifier with a L1 regularization, in which the training was on the 427 subjects using the TCGA gene abundance levels for the genes listed in FIG. 9B for which the feature is “gene expression” as well as TCGA mutation counts for the two genes in FIG. 9B for which the feature is “number of mutations.” In a third model, the classifier used was a support vector machine (SVM) classifier from Scikit-learn, as disclosed in Pedregosa et al. 2011, “Machine Learning in Python,” JMLR 12, pp. 2825-2830, hereby incorporated by reference, in which the training was on the 427 subjects but only using the TCGA gene abundance levels for the genes listed in FIG. 9B for which the feature is “gene expression.” When validated against data from a cohort of 133 subjects with cervical cancer or head and neck cancer and a known HPV status, the classifier performed with a specificity of 92.5% and a sensitivity of 89.7%.

In a fourth model, the classifier used was this same SVM classifier, in which the training was on the 427 subjects using the TCGA gene abundance levels for the genes listed in FIG. 9B for which the feature is “gene expression” as well as TCGA mutation counts for the two genes in FIG. 9B for which the feature is “number of mutations.” The performance of this trained classifier is reported in FIG. 9C. The regression coefficients and correlation statistics for each of the features used in the model are shown below in Tables 23 and 24, respectively. The SVM parameters used were class _weight: none, decision_function_shape: ovo, gamma: scale, kernel: linear, probability: True, shrinking: false, and tol: 1. As illustrated in FIG. 9C, the trained SVM predicts the cancer type of the 427 subjects, that is whether the subjects have the first cancer type (afflicted with HPV and having head and neck, or cervical cancer) or the second cancer type (not afflicted with HPV, but having head and neck, or cervical cancer) with a 99% specificity and 99% sensitivity for the training set of 427 subjects. The classifier was then validated against data from a cohort of 133 subjects with cervical cancer or head and neck cancer and a known HPV status. The classifier correctly identified the HPV infection status of 122 of the 133 validation subjects, with a specificity of 95% and a sensitivity of 87.5%.

TABLE 23

Regression coefficients for features used in the second SVM model for HPV detection

| Ensembl Gene ID | Gene Name | Feature Type | Coefficient |
|---|---|---|---|
| ENSG00000170442 | KRT86 | gene expression | 0.281204 |
| ENSG00000121005 | CRISPLD 1 | gene expression | 0.046559 |
| ENSG00000134760 | DSG1 | gene expression | 0.044229 |
| ENSG00000149212 | SESN3 | gene _expression | -0.26422 |
| ENSG00000173157 | ADAMTS20 | gene expression | -0.48575 |
| ENSG00000170549 | IRX1 | gene _expression | -0.09112 |
| ENSG00000077935 | SMC1B | gene expression | 1.020826 |
| ENSG00000147889 | CDKN2A | gene expression | 1.126704 |
| ENSG00000108947 | EFNB3 | gene expression | -0.97171 |
| ENSG00000145824 | CXCL14 | gene expression | -0.28714 |
| ENSG00000105278 | ZFR2 | gene expression | -0.00985 |
| ENSG00000178222 | RNF212 | gene expression | 0.517382 |
| ENSG00000179455 | MKRN3 | gene expression | -0.19302 |
| ENSG00000196074 | SYCP2 | gene expression | 0.315818 |
| ENSG00000168530 | MYL1 | gene expression | -0.15219 |
| ENSG00000095777 | MYO3A | gene expression | 0.465386 |
| ENSG00000182545 | RNASE10 | gene _expression | -0.36664 |
| ENSG00000144278 | GALNT13 | gene expression | -0.26314 |
| ENSG00000099625 | C 19orf26 | gene expression | -0.43544 |
| ENSG00000145113 | MUC4 | gene expression | -0.22115 |
| ENSG00000254221 | PCDHGB 1 | gene expression | -0.45707 |
| ENSG00000110092 | CCND1 | gene expression | -0.65063 |
| ENSG00000240386 | LCE1F | gene expression | 0.198233 |
| ENSG00000124134 | KCNS1 | gene expression | 0.7377 |
| TP53 | TP53 | mutational status | -0.4517 |
| CDKN2A | CDKN2A | mutational status | -0.26302 |

TABLE 24

Correlation statistics for the features used in the second SVM model for HPV detection

| Feature 1 | Feature 2 | Correlation | Highly Correlated Pair # |
|---|---|---|---|
| ENSG00000121005 | ENSG00000170442 | -0.04066 | |
| ENSG00000134760 | ENSG00000170442 | -0.1313 | |
| ENSG00000134760 | ENSG00000121005 | 0.134678 | |
| ENSG00000149212 | ENSG00000170442 | -0.25182 | |
| ENSG00000149212 | ENSG00000121005 | 0.488664 | |
| ENSG00000149212 | ENSG00000134760 | 0.355098 | |
| ENSG00000173157 | ENSG00000170442 | 0.061926 | |
| ENSG00000173157 | ENSG00000121005 | 0.506442 | |
| ENSG00000173157 | ENSG00000134760 | 0.090731 | |
| ENSG00000173157 | ENSG00000149212 | 0.275716 | |
| ENSG00000170549 | ENSG00000170442 | -0.05431 | |
| ENSG00000170549 | ENSG00000121005 | 0.297916 | |
| ENSG00000170549 | ENSG00000134760 | 0.390033 | |
| ENSG00000170549 | ENSG00000149212 | 0.16815 | |
| ENSG00000170549 | ENSG00000173157 | 0.190158 | |
| ENSG00000077935 | ENSG00000170442 | 0.508903 | |
| ENSG00000077935 | ENSG00000121005 | -0.21228 | |
| ENSG00000077935 | ENSG00000134760 | -0.28965 | |
| ENSG00000077935 | ENSG00000149212 | -0.32522 | |
| ENSG00000077935 | ENSG00000173157 | -0.09144 | |
| ENSG00000077935 | ENSG00000170549 | -0.33638 | |
| ENSG00000147889 | ENSG00000170442 | 0.249512 | |
| ENSG00000147889 | ENSG00000121005 | -0.1551 | |
| ENSG00000147889 | ENSG00000134760 | -0.05004 | |
| ENSG00000147889 | ENSG00000149212 | 0.011617 | |
| ENSG00000147889 | ENSG00000173157 | -0.05178 | |
| ENSG00000147889 | ENSG00000170549 | -0.23241 | |
| ENSG00000147889 | ENSG00000077935 | 0.562316 | |
| ENSG00000108947 | ENSG00000170442 | -0.03695 | |
| ENSG00000108947 | ENSG00000121005 | 0.324505 | |
| ENSG00000108947 | ENSG00000134760 | 0.040914 | |
| ENSG00000108947 | ENSG00000149212 | 0.141273 | |
| ENSG00000108947 | ENSG00000173157 | 0.240437 | |
| ENSG00000108947 | ENSG00000170549 | 0.365244 | |
| ENSG00000108947 | ENSG00000077935 | -0.22954 | |
| ENSG00000108947 | ENSG00000147889 | -0.29009 | |
| ENSG00000145824 | ENSG00000170442 | 0.069094 | |
| ENSG00000145824 | ENSG00000121005 | 0.248397 | |
| ENSG00000145824 | ENSG00000134760 | 0.601905 | 1 |
| ENSG00000145824 | ENSG00000149212 | 0.181146 | |
| ENSG00000145824 | ENSG00000173157 | 0.192195 | |
| ENSG00000145824 | ENSG00000170549 | 0.461357 | |
| ENSG00000145824 | ENSG00000077935 | -0.2336 | |
| ENSG00000145824 | ENSG00000147889 | -0.11632 | |
| ENSG00000145824 | ENSG00000108947 | 0.261769 | |
| ENSG00000105278 | ENSG00000170442 | 0.250168 | |
| ENSG00000105278 | ENSG00000121005 | -0.12744 | |
| ENSG00000105278 | ENSG00000134760 | -0.2786 | |
| ENSG00000105278 | ENSG00000149212 | -0.08982 | |
| ENSG00000105278 | ENSG00000173157 | -0.06139 | |
| ENSG00000105278 | ENSG00000170549 | -0.22704 | |
| ENSG00000105278 | ENSG00000077935 | 0.718983 | 2 |
| ENSG00000105278 | ENSG00000147889 | 0.490566 | |
| ENSG00000105278 | ENSG00000108947 | -0.08563 | |
| ENSG00000105278 | ENSG00000145824 | -0.29907 | |
| ENSG00000178222 | ENSG00000170442 | 0.317245 | |
| ENSG00000178222 | ENSG00000121005 | -0.14501 | |
| ENSG00000178222 | ENSG00000134760 | -0.10005 | |
| ENSG00000178222 | ENSG00000149212 | -0.18412 | |
| ENSG00000178222 | ENSG00000173157 | -0.11824 | |
| ENSG00000178222 | ENSG00000170549 | -0.15257 | |
| ENSG00000178222 | ENSG00000077935 | 0.649568 | 3 |
| ENSG00000178222 | ENSG00000147889 | 0.460545 | |
| ENSG00000178222 | ENSG00000108947 | -0.12628 | |
| ENSG00000178222 | ENSG00000145824 | -0.01065 | |
| ENSG00000178222 | ENSG00000105278 | 0.495493 | |
| ENSG00000179455 | ENSG00000170442 | 0.140679 | |
| ENSG00000179455 | ENSG00000121005 | 0.420858 | |
| ENSG00000179455 | ENSG00000134760 | 0.160431 | |
| ENSG00000179455 | ENSG00000149212 | 0.267878 | |
| ENSG00000179455 | ENSG00000173157 | 0.353586 | |
| ENSG00000179455 | ENSG00000170549 | 0.222223 | |
| ENSG00000179455 | ENSG00000077935 | 0.018466 | |
| ENSG00000179455 | ENSG00000147889 | -0.04649 | |
| ENSG00000179455 | ENSG00000108947 | 0.223497 | |
| ENSG00000179455 | ENSG00000145824 | 0.236049 | |
| ENSG00000179455 | ENSG00000105278 | 0.078913 | |
| ENSG00000179455 | ENSG00000178222 | -0.00614 | |
| ENSG00000196074 | ENSG00000170442 | 0.416286 | |
| ENSG00000196074 | ENSG00000121005 | -0.17789 | |
| ENSG00000196074 | ENSG00000134760 | -0.28147 | |
| ENSG00000196074 | ENSG00000149212 | -0.14735 | |
| ENSG00000196074 | ENSG00000173157 | -0.10223 | |
| ENSG00000196074 | ENSG00000170549 | -0.35681 | |
| ENSG00000196074 | ENSG00000077935 | 0.800768 | 4 |
| ENSG00000196074 | ENSG00000147889 | 0.512305 | |
| ENSG00000196074 | ENSG00000108947 | -0.28738 | |
| ENSG00000196074 | ENSG00000145824 | -0.33066 | |
| ENSG00000196074 | ENSG00000105278 | 0.648232 | 5 |
| ENSG00000196074 | ENSG00000178222 | 0.593545 | |
| ENSG00000196074 | ENSG00000179455 | 0.016211 | |
| ENSG00000168530 | ENSG00000170442 | 0.099129 | |
| ENSG00000168530 | ENSG00000121005 | 0.284863 | |
| ENSG00000168530 | ENSG00000134760 | 0.284947 | |
| ENSG00000168530 | ENSG00000149212 | 0.07944 | |
| ENSG00000168530 | ENSG00000173157 | 0.190962 | |
| ENSG00000168530 | ENSG00000170549 | 0.32725 | |
| ENSG00000168530 | ENSG00000077935 | -0.06582 | |
| ENSG00000168530 | ENSG00000147889 | -0.02298 | |
| ENSG00000168530 | ENSG00000108947 | 0.085707 | |
| ENSG00000168530 | ENSG00000145824 | 0.389225 | |
| ENSG00000168530 | ENSG00000105278 | -0.07999 | |
| ENSG00000168530 | ENSG00000178222 | -0.02681 | |
| ENSG00000168530 | ENSG00000179455 | 0.277902 | |
| ENSG00000168530 | ENSG00000196074 | -0.12664 | |
| ENSG00000095777 | ENSG00000170442 | 0.338683 | |
| ENSG00000095777 | ENSG00000121005 | -0.05498 | |
| ENSG00000095777 | ENSG00000134760 | -0.21963 | |
| ENSG00000095777 | ENSG00000149212 | -0.14035 | |
| ENSG00000095777 | ENSG00000173157 | -0.00022 | |
| ENSG00000095777 | ENSG00000170549 | -0.28482 | |
| ENSG00000095777 | ENSG00000077935 | 0.613609 | 6 |
| ENSG00000095777 | ENSG00000147889 | 0.473209 | |
| ENSG00000095777 | ENSG00000108947 | -0.20146 | |
| ENSG00000095777 | ENSG00000145824 | -0.27264 | |
| ENSG00000095777 | ENSG00000105278 | 0.531262 | |
| ENSG00000095777 | ENSG00000178222 | 0.464102 | |
| ENSG00000095777 | ENSG00000179455 | 0.018963 | |
| ENSG00000095777 | ENSG00000196074 | 0.659032 | 7 |
| ENSG00000095777 | ENSG00000168530 | -0.05023 | |
| ENSG00000182545 | ENSG00000170442 | 0.192319 | |
| ENSG00000182545 | ENSG00000121005 | 0.196649 | |
| ENSG00000182545 | ENSG00000134760 | 0.179965 | |
| ENSG00000182545 | ENSG00000149212 | 0.053477 | |
| ENSG00000182545 | ENSG00000173157 | 0.296745 | |
| ENSG00000182545 | ENSG00000170549 | 0.136928 | |
| ENSG00000182545 | ENSG00000077935 | 0.084728 | |
| ENSG00000182545 | ENSG00000147889 | 0.050558 | |
| ENSG00000182545 | ENSG00000108947 | 0.095014 | |
| ENSG00000182545 | ENSG00000145824 | 0.221964 | |
| ENSG00000182545 | ENSG00000105278 | 0.008214 | |
| ENSG00000182545 | ENSG00000178222 | 0.048557 | |
| ENSG00000182545 | ENSG00000179455 | 0.246635 | |
| ENSG00000182545 | ENSG00000196074 | -0.01025 | |
| ENSG00000182545 | ENSG00000168530 | 0.140587 | |
| ENSG00000182545 | ENSG00000095777 | 0.017852 | |
| ENSG00000144278 | ENSG00000170442 | -0.00696 | |
| ENSG00000144278 | ENSG00000121005 | 0.437315 | |
| ENSG00000144278 | ENSG00000134760 | 0.075964 | |
| ENSG00000144278 | ENSG00000149212 | 0.34696 | |
| ENSG00000144278 | ENSG00000173157 | 0.354405 | |
| ENSG00000144278 | ENSG00000170549 | 0.299819 | |
| ENSG00000144278 | ENSG00000077935 | -0.20079 | |
| ENSG00000144278 | ENSG00000147889 | -0.04385 | |
| ENSG00000144278 | ENSG00000108947 | 0.247868 | |
| ENSG00000144278 | ENSG00000145824 | 0.219262 | |
| ENSG00000144278 | ENSG00000105278 | -0.07425 | |
| ENSG00000144278 | ENSG00000178222 | -0.06659 | |
| ENSG00000144278 | ENSG00000179455 | 0.329653 | |
| ENSG00000144278 | ENSG00000196074 | -0.15614 | |
| ENSG00000144278 | ENSG00000168530 | 0.187905 | |
| ENSG00000144278 | ENSG00000095777 | -0.14318 | |
| ENSG00000144278 | ENSG00000182545 | 0.037964 | |
| ENSG00000099625 | ENSG00000170442 | -0.08444 | |
| ENSG00000099625 | ENSG00000121005 | 0.290868 | |
| ENSG00000099625 | ENSG00000134760 | 0.195054 | |
| ENSG00000099625 | ENSG00000149212 | 0.277271 | |
| ENSG00000099625 | ENSG00000173157 | 0.277417 | |
| ENSG00000099625 | ENSG00000170549 | 0.354007 | |
| ENSG00000099625 | ENSG00000077935 | -0.14724 | |
| ENSG00000099625 | ENSG00000147889 | -0.07707 | |
| ENSG00000099625 | ENSG00000108947 | 0.562589 | |
| ENSG00000099625 | ENSG00000145824 | 0.190164 | |
| ENSG00000099625 | ENSG00000105278 | 0.027462 | |
| ENSG00000099625 | ENSG00000178222 | -0.14514 | |
| ENSG00000099625 | ENSG00000179455 | 0.241907 | |
| ENSG00000099625 | ENSG00000196074 | -0.21507 | |
| ENSG00000099625 | ENSG00000168530 | 0.211523 | |
| ENSG00000099625 | ENSG00000095777 | -0.19116 | |
| ENSG00000099625 | ENSG00000182545 | 0.209451 | |
| ENSG00000099625 | ENSG00000144278 | 0.343114 | |
| ENSG00000145113 | ENSG00000170442 | 0.458215 | |
| ENSG00000145113 | ENSG00000121005 | -0.18624 | |
| ENSG00000145113 | ENSG00000134760 | -0.18101 | |
| ENSG00000145113 | ENSG00000149212 | -0.483 | |
| ENSG00000145113 | ENSG00000173157 | -0.05284 | |
| ENSG00000145113 | ENSG00000170549 | -0.13827 | |
| ENSG00000145113 | ENSG00000077935 | 0.523288 | |
| ENSG00000145113 | ENSG00000147889 | 0.26829 | |
| ENSG00000145113 | ENSG00000108947 | -0.07115 | |
| ENSG00000145113 | ENSG00000145824 | 0.041071 | |
| ENSG00000145113 | ENSG00000105278 | 0.299568 | |
| ENSG00000145113 | ENSG00000178222 | 0.364255 | |
| ENSG00000145113 | ENSG00000179455 | 0.056978 | |
| ENSG00000145113 | ENSG00000196074 | 0.350754 | |
| ENSG00000145113 | ENSG00000168530 | 0.075096 | |
| ENSG00000145113 | ENSG00000095777 | 0.323163 | |
| ENSG00000145113 | ENSG00000182545 | 0.241423 | |
| ENSG00000145113 | ENSG00000144278 | -0.1955 | |
| ENSG00000145113 | ENSG00000099625 | -0.11693 | |
| ENSG00000254221 | ENSG00000170442 | 0.003591 | |
| ENSG00000254221 | ENSG00000121005 | 0.435801 | |
| ENSG00000254221 | ENSG00000134760 | 0.007706 | |
| ENSG00000254221 | ENSG00000149212 | 0.324084 | |
| ENSG00000254221 | ENSG00000173157 | 0.334907 | |
| ENSG00000254221 | ENSG00000170549 | 0.256845 | |
| ENSG00000254221 | ENSG00000077935 | -0.18828 | |
| ENSG00000254221 | ENSG00000147889 | -0.1212 | |
| ENSG00000254221 | ENSG00000108947 | 0.437106 | |
| ENSG00000254221 | ENSG00000145824 | 0.125222 | |
| ENSG00000254221 | ENSG00000105278 | -0.12422 | |
| ENSG00000254221 | ENSG00000178222 | -0.09784 | |
| ENSG00000254221 | ENSG00000179455 | 0.311361 | |
| ENSG00000254221 | ENSG00000196074 | -0.14597 | |
| ENSG00000254221 | ENSG00000168530 | 0.090272 | |
| ENSG00000254221 | ENSG00000095777 | -0.19747 | |
| ENSG00000254221 | ENSG00000182545 | 0.116585 | |
| ENSG00000254221 | ENSG00000144278 | 0.45402 | |
| ENSG00000254221 | ENSG00000099625 | 0.325875 | |
| ENSG00000254221 | ENSG00000145113 | -0.19429 | |
| ENSG00000110092 | ENSG00000170442 | 0.215807 | |
| ENSG00000110092 | ENSG00000121005 | 0.186991 | |
| ENSG00000110092 | ENSG00000134760 | 0.078778 | |
| ENSG00000110092 | ENSG00000149212 | -0.18427 | |
| ENSG00000110092 | ENSG00000173157 | 0.182797 | |
| ENSG00000110092 | ENSG00000170549 | 0.36607 | |
| ENSG00000110092 | ENSG00000077935 | -0.05316 | |
| ENSG00000110092 | ENSG00000147889 | -0.19008 | |
| ENSG00000110092 | ENSG00000108947 | 0.453148 | |
| ENSG00000110092 | ENSG00000145824 | 0.34624 | |
| ENSG00000110092 | ENSG00000105278 | -0.08277 | |
| ENSG00000110092 | ENSG00000178222 | -0.16028 | |
| ENSG00000110092 | ENSG00000179455 | 0.212791 | |
| ENSG00000110092 | ENSG00000196074 | -0.22647 | |
| ENSG00000110092 | ENSG00000168530 | 0.234684 | |
| ENSG00000110092 | ENSG00000095777 | -0.07161 | |
| ENSG00000110092 | ENSG00000182545 | 0.262054 | |
| ENSG00000110092 | ENSG00000144278 | 0.098067 | |
| ENSG00000110092 | ENSG00000099625 | 0.409195 | |
| ENSG00000110092 | ENSG00000145113 | 0.357647 | |
| ENSG00000110092 | ENSG00000254221 | 0.157465 | |
| ENSG00000240386 | ENSG00000170442 | -0.12567 | |
| ENSG00000240386 | ENSG00000121005 | 0.11863 | |
| ENSG00000240386 | ENSG00000134760 | 0.672628 | 8 |
| ENSG00000240386 | ENSG00000149212 | 0.253078 | |
| ENSG00000240386 | ENSG00000173157 | 0.191005 | |
| ENSG00000240386 | ENSG00000170549 | 0.469055 | |
| ENSG00000240386 | ENSG00000077935 | -0.34989 | |
| ENSG00000240386 | ENSG00000147889 | -0.1204 | |
| ENSG00000240386 | ENSG00000108947 | 0.21399 | |
| ENSG00000240386 | ENSG00000145824 | 0.571567 | |
| ENSG00000240386 | ENSG00000105278 | -0.25585 | |
| ENSG00000240386 | ENSG00000178222 | -0.16551 | |
| ENSG00000240386 | ENSG00000179455 | 0.103887 | |
| ENSG00000240386 | ENSG00000196074 | -0.35606 | |
| ENSG00000240386 | ENSG00000168530 | 0.295515 | |
| ENSG00000240386 | ENSG00000095777 | -0.29516 | |
| ENSG00000240386 | ENSG00000182545 | 0.198916 | |
| ENSG00000240386 | ENSG00000144278 | 0.095936 | |
| ENSG00000240386 | ENSG00000099625 | 0.288385 | |
| ENSG00000240386 | ENSG00000145113 | -0.18358 | |
| ENSG00000240386 | ENSG00000254221 | 0.080361 | |
| ENSG00000240386 | ENSG00000110092 | 0.233552 | |
| ENSG00000124134 | ENSG00000170442 | 0.323343 | |
| ENSG00000124134 | ENSG00000121005 | -0.23394 | |
| ENSG00000124134 | ENSG00000134760 | -0.07179 | |
| ENSG00000124134 | ENSG00000149212 | -0.15515 | |
| ENSG00000124134 | ENSG00000173157 | -0.12997 | |
| ENSG00000124134 | ENSG00000170549 | -0.22963 | |
| ENSG00000124134 | ENSG00000077935 | 0.693565 | 9 |
| ENSG00000124134 | ENSG00000147889 | 0.545043 | |
| ENSG00000124134 | ENSG00000108947 | -0.2682 | |
| ENSG00000124134 | ENSG00000145824 | -0.09267 | |
| ENSG00000124134 | ENSG00000105278 | 0.616996 | 10 |
| ENSG00000124134 | ENSG00000178222 | 0.514734 | |
| ENSG00000124134 | ENSG00000179455 | 0.011375 | |
| ENSG00000124134 | ENSG00000196074 | 0.599981 | |
| ENSG00000124134 | ENSG00000168530 | 0.052773 | |
| ENSG00000124134 | ENSG00000095777 | 0.414669 | |
| ENSG00000124134 | ENSG00000182545 | 0.073025 | |
| ENSG00000124134 | ENSG00000144278 | -0.14665 | |
| ENSG00000124134 | ENSG00000099625 | -0.02252 | |
| ENSG00000124134 | ENSG00000145113 | 0.399469 | |
| ENSG00000124134 | ENSG00000254221 | -0.18319 | |
| ENSG00000124134 | ENSG00000110092 | -0.04119 | |
| ENSG00000124134 | ENSG00000240386 | -0.12953 | |
| TP53 | ENSG00000170442 | -0.203 | |
| TP53 | ENSG00000121005 | 0.171477 | |
| TP53 | ENSG00000134760 | 0.349983 | |
| TP53 | ENSG00000149212 | 0.220628 | |
| TP53 | ENSG00000173157 | 0.224804 | |
| TP53 | ENSG00000170549 | 0.322259 | |
| TP53 | ENSG00000077935 | -0.42909 | |
| TP53 | ENSG00000147889 | -0.15848 | |
| TP53 | ENSG00000108947 | 0.14238 | |
| TP53 | ENSG00000145824 | 0.289419 | |
| TP53 | ENSG00000105278 | -0.33551 | |
| TP53 | ENSG00000178222 | -0.26775 | |
| TP53 | ENSG00000179455 | 0.129312 | |
| TP53 | ENSG00000196074 | -0.40505 | |
| TP53 | ENSG00000168530 | 0.147047 | |
| TP53 | ENSG00000095777 | -0.29804 | |
| TP53 | ENSG00000182545 | 0.16223 | |
| TP53 | ENSG00000144278 | 0.296668 | |
| TP53 | ENSG00000099625 | 0.18133 | |
| TP53 | ENSG00000145113 | -0.18051 | |
| TP53 | ENSG00000254221 | 0.165337 | |
| TP53 | ENSG00000110092 | 0.109177 | |
| TP53 | ENSG00000240386 | 0.383618 | |
| TP53 | ENSG00000124134 | -0.32845 | |
| CDKN2A | ENSG00000170442 | -0.14855 | |
| CDKN2A | ENSG00000121005 | 0.088698 | |
| CDKN2A | ENSG00000134760 | 0.19446 | |
| CDKN2A | ENSG00000149212 | 0.191928 | |
| CDKN2A | ENSG00000173157 | 0.153285 | |
| CDKN2A | ENSG00000170549 | 0.231313 | |
| CDKN2A | ENSG00000077935 | -0.27452 | |
| CDKN2A | ENSG00000147889 | 0.056256 | |
| CDKN2A | ENSG00000108947 | 0.060295 | |
| CDKN2A | ENSG00000145824 | 0.121151 | |
| CDKN2A | ENSG00000105278 | -0.25297 | |
| CDKN2A | ENSG00000178222 | -0.20681 | |
| CDKN2A | ENSG00000179455 | 0.068506 | |
| CDKN2A | ENSG00000196074 | -0.2943 | |
| CDKN2A | ENSG00000168530 | 0.149041 | |
| CDKN2A | ENSG00000095777 | -0.21265 | |
| CDKN2A | ENSG00000182545 | 0.140598 | |
| CDKN2A | ENSG00000144278 | 0.120321 | |
| CDKN2A | ENSG00000099625 | 0.093298 | |
| CDKN2A | ENSG00000145113 | -0.17281 | |
| CDKN2A | ENSG00000254221 | 0.19745 | |
| CDKN2A | ENSG00000110092 | 0.00086 | |
| CDKN2A | ENSG00000240386 | 0.205975 | |
| CDKN2A | ENSG00000124134 | -0.25407 | |
| CDKN2A | TP53 | 0.436135 | |

To validate the model, the trained SVM classifier reported in FIG. 9C was tested against a validation population that had not been used to train the classifier. As detailed in FIG. 9A, the validation dataset comprised a corresponding plurality of abundance values for each subject in a dataset termed the “Testing” dataset, described in Example 7, that had cervical cancer or head and neck cancer with known HPV status. As illustrated in FIG. 9A, 133 subjects from the validation dataset were selected who satisfied these selection criteria and served as the plurality of subjects of the validation dataset. Of the 133 validation subjects, 93 had head and neck cancer and 40 had cervical cancer. Of the 93 subjects that had head and neck cancer, 28 tested positive for HPV and 65 tested negative for HPV. Of the 40 subjects that had cervical cancer, 28 tested positive for HPV and 12 tested negative for HPV. Thus, of the 133 validation subjects, 56 validation subjects were deemed to have the first cancer condition (afflicted with HPV and having head and neck, or cervical cancer) and the remaining 77 validation subjects (not afflicted with HPV, but having head and neck, or cervical cancer) were deemed to have the second cancer condition.

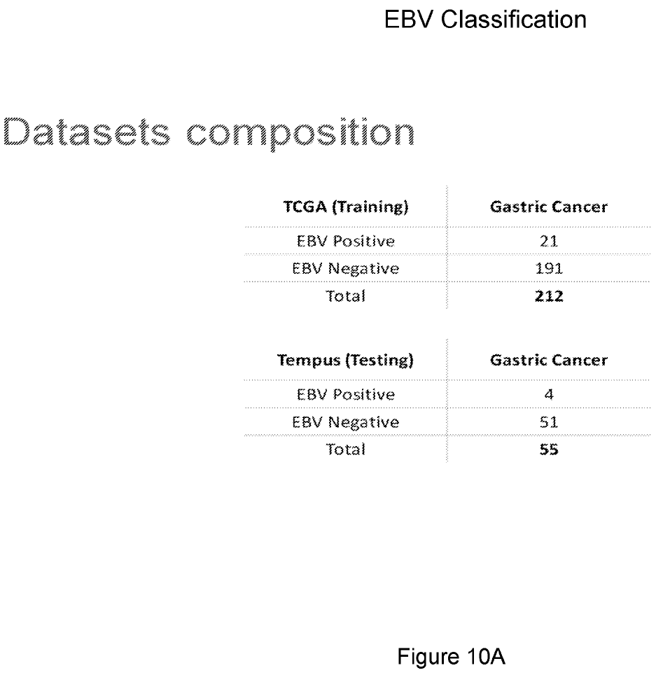

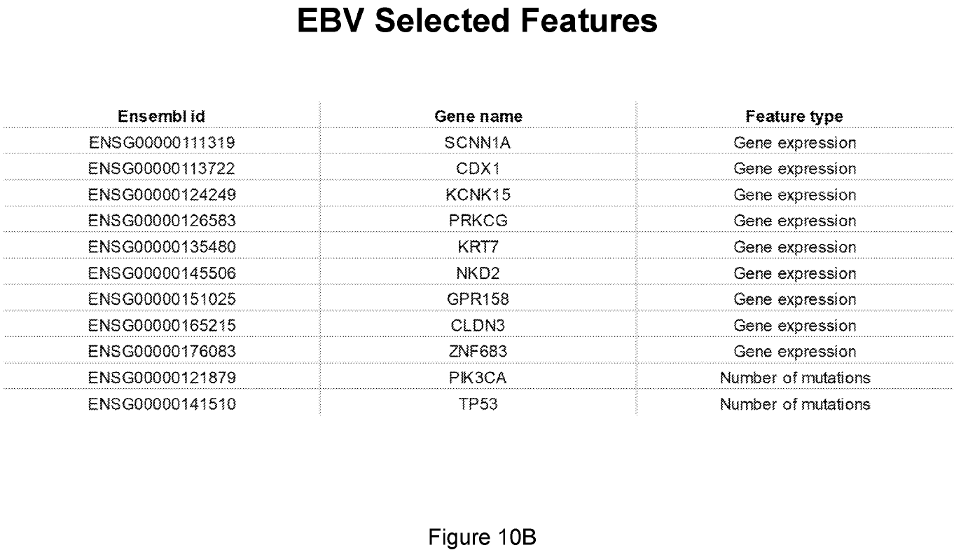

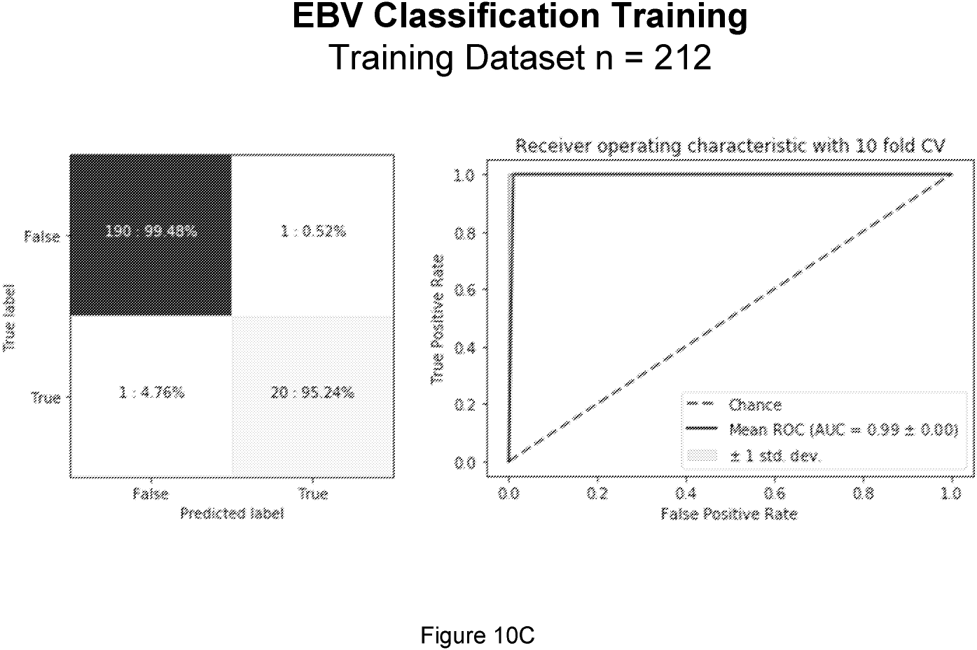

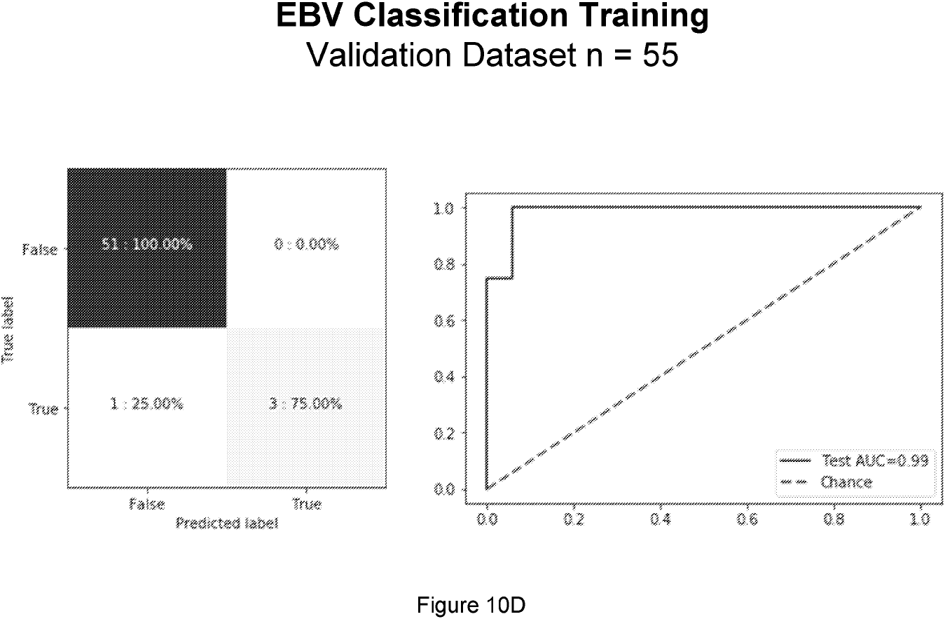

Each of the 133 validation subjects were run against the trained SVM whose performance is reported in FIG. 9C and thus was assigned by the SVM to either the first or second cancer class. That is, the gene abundance values for the genes listed in FIG. 9B in which the feature type was “gene expression” and the mutation count in the two genes listed in FIG. 9B in which the feature type was “number of mutations” was measured from a tumor sample for each of the 133 validation subjects and this data for each validation subject was separately input into the trained SVM model of FIG. 10C. As illustrated in FIG. 9D, the trained SVM had 95% specificity and 88% sensitivity for cancer class across the 133 validation subjects. It was found that the addition of the covariate of the number of mutations in the genes TP53 and CDKN2A to the SVM doesn’t change the accuracy but improves the AUC from 0.97 to 0.98. This example shows that the trained SVM model accurately predicts viral infection in tumors using RNA expression data.

This example confirms viral infections are generally associated with an upregulation of immune responses. This example further shows that viral detection based on whole transcriptome data is a useful clinical tool in its own right, and further can be combined with existing diagnostic methods to provide insights about the viral status and tumor microenvironment in a single test.

### Example 9 – Epstein Barr Virus Detection

Referring to FIGS. 10A through 10D, a classifier for determining EBV viral status was trained using gene expression from the tumor RNA-seq data of a training population, where each subject in the training population had been diagnosed with gastric cancer.

In accordance with block 1204 of FIG. 7A, the training dataset was obtained. Here, the dataset comprised a corresponding plurality of abundance values for each subject in the TCGA, described in Example 1, that had gastric cancer with known EBV status. As illustrated in FIG. 10A, there were 212 subjects in the TCGA that satisfied these selection criteria and thus served as the plurality of subjects of the training dataset. Of the 212 subjects, 21 tested positive for EBV and 191 tested negative for EBV. Thus, of the 212 subjects, 21 subjects were deemed to have the first cancer condition (afflicted with EBV and having gastric cancer) and the remaining 191 subjects were deemed to have the second cancer condition (not afflicted with EBV, but having gastric cancer).

Next, in accordance with block 1218 of FIG. 7C and block 228 of FIG. 7D, the gene expression values from whole exome RNA data in the TCGA dataset for the 212 subjects was used to identify a discriminating gene set by regression, in which the gene expression values obtained from whole exome mRNA expression data for the 212 subjects in the TCGA dataset served as independent variables and the indication of whether a respective subject had the first cancer condition (afflicted with EBV and having gastric cancer) or the second cancer condition (not afflicted with EBV, but having gastric cancer) served as the dependent variable. More specifically, in accordance with block 1228 of FIG. 7D, the dataset consisting of 212 subjects was split into ten sets (ten splits). Each set included two or more subjects afflicted with the first cancer condition and two or more subjects afflicted with the second cancer condition. Each respective set of the ten sets (splits) was independently subjected to regression in which whole exome mRNA expression data for the subjects of the respective set served as independent variables and the indication of whether a respective subject in the respective set had the first or second cancer condition served as the dependent variable. Each regression (split) was performed with L1 (LASSO) regularization in accordance with block 1238 of FIG. 7E. Since L1 regularization leads to sparse coefficients, only a small subset of genes had non-zero coefficients for each set. Only the genes with non-zero coefficients in more than 80% of the sets were included in the final model. In other words, only those genes that had non-zero regression coefficients for at least eight of the ten sets (splits) were accepted into the discriminating set of genes on the basis of their expression data. The list of genes that satisfied this requirement are the ones listed in FIG. 10B in which the feature type is “gene expression.” Furthermore, FIG. 11B illustrates principal component analysis of the abundance values of the genes listed in FIG. 10B across the training set. FIG. 11B illustrates that a plot of the first and second PCA values for each of the subjects in the training set break out into two distinct groups, corresponding to the first cancer condition (group 1606) and second cancer condition (1606), indicating the power of the abundance values of the genes listed in FIG. 10B to discriminate between the first and second cancer state.

In some embodiments, additional genes were included in the discriminating set of genes based on the presence or absence of mutations (e.g., the number of mutations) in the additional genes. In this example, as detailed in FIG. 10B, the genes PIK3CA and TP53 were included in the discriminating set of genes and the feature for these genes was the number of times mutations were observed in these genes in each of the respective 212 subjects of the training set.

Next, in accordance with block 1242 of FIG. 7E, the respective abundance values for the discriminating gene set and the respective indication of cancer condition across the 212 subjects was used to train a classifier to discriminate between the first and second cancer conditions as a function of respective abundance values for the discriminating gene set. In a first model, the classifier used was a logistic regression classifier with a L1 regularization, in which the training was the 212 subjects but only using TCGA gene abundance levels for the genes listed in FIG. 10B for which the feature is “gene expression.” In a second model, the classifier used was a logistic regression classifier with a L1 regularization, in which the training was on the 212 subjects using the TCGA gene abundance levels for the genes listed in FIG. 10B for which the feature is “gene expression” as well as TCGA mutation counts for the two genes in FIG. 10B for which the feature is “number of mutations.” In a third model, the classifier used was a support vector machine (SVM) classifier from Scikit-learn, as disclosed in Pedregosa et al. 2011, “Machine Learning in Python,” JMLR 12, pp. 2825-2830, hereby incorporated by reference, in which the training was on the 212 subjects but only using the TCGA gene abundance levels for the genes listed in FIG. 10B for which the feature is “gene expression.” When validated against data from a cohort of 55 subjects with gastric cancer and a known EBV status, the classifier correctly identified the EBV infection status of 54 or the 55 validation subjects, with a specificity of 100% and a sensitivity of 75%.

In a fourth model, the classifier used was this same SVM classifier, in which the training was on the 212 subjects and using the TCGA gene abundance levels for the genes listed in FIG. 9B for which the feature is “gene expression” as well as TCGA mutation counts for the two genes in FIG. 9B for which the feature is “number of mutations.” The performance of this trained classifier is reported in FIG. 10C. The regression coefficients and correlation statistics for each of the features used in the model are shown below in Tables 25 and 26, respectively. The SVM parameters used were class_weight: none, decision_function_shape: ovo, gamma: scale, kernel: linear, probability: True, shrinking: false, and tol: 1. As illustrated in FIG. 10C, the trained SVM predicts the cancer type of the 212 subjects, that is whether the subjects have the first cancer type (afflicted with EBV and having gastric cancer) or the second cancer type (not afflicted with EBV, but having gastric cancer) with a 99% specificity and 95% sensitivity for the training set of 212 subjects. The classifier was then validated against data from a cohort of 55 subjects with gastric cancer and a known EBV status. The classifier correctly identified the EBV infection status of 54 of the 55 validation subjects, with a specificity of 100% and a sensitivity of 75%.

TABLE 25

Regression coefficients for features used in the second SVM model for EBV detection

| Ensembl Gene ID | Gene Name | Feature Type | Coefficient |
|---|---|---|---|
| ENSG00000111319 | SCNN1A | gene_expression | -1.2572 |
| ENSG00000113722 | CDX1 | gene_expression | -0.66772 |
| ENSG00000124249 | KCNK15 | gene_expression | -1.04267 |
| ENSG00000126583 | PRKCG | gene_expression | 0.63421 |
| ENSG00000135480 | KRT7 | gene_expression | -0.94353 |
| ENSG00000145506 | NKD2 | gene_expression | -0.66031 |
| ENSG00000151025 | GPR158 | gene_expression | -0.62359 |
| ENSG00000165215 | CLDN3 | gene_expression | -1.67826 |
| ENSG00000176083 | ZNF683 | gene_expression | 0.592752 |
| TP53 | TP53 | mutational_status | -0.61494 |
| PIK3CA | PIK3CA | mutational_status | 0.520923 |

TABLE 26

Correlation statistics for the features used in the second SVM model for EBV detection

| Feature 1 | Feature 2 | Correlation |
|---|---|---|
| ENSG00000113722 | ENSG00000111319 | 0.104724 |
| ENSG00000124249 | ENSG00000111319 | 0.429128 |
| ENSG00000124249 | ENSG00000113722 | -0.20282 |
| ENSG00000126583 | ENSG00000111319 | -0.16662 |
| ENSG00000126583 | ENSG00000113722 | 0.11953 |
| ENSG00000126583 | ENSG00000124249 | -0.14871 |
| ENSG00000135480 | ENSG00000111319 | 0.452307 |
| ENSG00000135480 | ENSG00000113722 | -0.42786 |
| ENSG00000135480 | ENSG00000124249 | 0.650944 |
| ENSG00000135480 | ENSG00000126583 | -0.10185 |
| ENSG00000145506 | ENSG00000111319 | -0.12667 |
| ENSG00000145506 | ENSG00000113722 | 0.051531 |
| ENSG00000145506 | ENSG00000124249 | 0.109441 |
| ENSG00000145506 | ENSG00000126583 | -0.19096 |
| ENSG00000145506 | ENSG00000135480 | -0.01553 |
| ENSG00000151025 | ENSG00000111319 | 0.174624 |
| ENSG00000151025 | ENSG00000113722 | -0.03132 |
| ENSG00000151025 | ENSG00000124249 | 0.187233 |
| ENSG00000151025 | ENSG00000126583 | -0.20936 |
| ENSG00000151025 | ENSG00000135480 | 0.131621 |
| ENSG00000151025 | ENSG00000145506 | 0.001804 |
| ENSG00000165215 | ENSG00000111319 | 0.264786 |
| ENSG00000165215 | ENSG00000113722 | 0.578454 |
| ENSG00000165215 | ENSG00000124249 | 0.22998 |
| ENSG00000165215 | ENSG00000126583 | -0.02774 |
| ENSG00000165215 | ENSG00000135480 | 0.048908 |
| ENSG00000165215 | ENSG00000145506 | 0.005267 |
| ENSG00000165215 | ENSG00000151025 | 0.009025 |
| ENSG00000176083 | ENSG00000111319 | 0.028252 |
| ENSG00000176083 | ENSG00000113722 | -0.16096 |
| ENSG00000176083 | ENSG00000124249 | -0.24414 |
| ENSG00000176083 | ENSG00000126583 | 0.147816 |
| ENSG00000176083 | ENSG00000135480 | -0.10308 |
| ENSG00000176083 | ENSG00000145506 | 0.029865 |
| ENSG00000176083 | ENSG00000151025 | -0.12438 |
| ENSG00000176083 | ENSG00000165215 | -0.2766 |
| TP53 | ENSG00000111319 | 0.11033 |
| TP53 | ENSG00000113722 | -0.00053 |
| TP53 | ENSG00000124249 | 0.157624 |
| TP53 | ENSG00000126583 | -0.2485 |
| TP53 | ENSG00000135480 | 0.17002 |
| TP53 | ENSG00000145506 | 0.164913 |
| TP53 | ENSG00000151025 | 0.185344 |
| TP53 | ENSG00000165215 | 0.309497 |
| TP53 | ENSG00000176083 | -0.05715 |
| PIK3CA | ENSG00000111319 | -0.36062 |
| PIK3CA | ENSG00000113722 | -0.10222 |
| PIK3CA | ENSG00000124249 | -0.20278 |
| PIK3CA | ENSG00000126583 | 0.29328 |
| PIK3CA | ENSG00000135480 | -0.34703 |
| PIK3CA | ENSG00000145506 | -0.15388 |
| PIK3CA | ENSG00000151025 | -0.23884 |
| PIK3CA | ENSG00000165215 | -0.11482 |
| PIK3CA | ENSG00000176083 | 0.04957 |
| PIK3CA | TP53 | -0.10617 |

To validate the model, the trained SVM classifier reported in FIG. 10C was tested against a validation population that had not been used to train the classifier. As detailed in FIG. 10A, the validation dataset comprised a corresponding plurality of abundance values for each subject in a dataset termed the “Testing” dataset, described in Example 2, that had gastric cancer with known EBV status. As illustrated in FIGS. 10A, 55 subjects were selected from the validation dataset that satisfied these selection criteria and served as the plurality of subjects of the validation dataset. Of the 55 validation subjects, 4 tested positive for EBV and 51 tested negative for EBV. Thus, of the 55 validation subjects, 4 validation subjects were deemed to have the first cancer condition (afflicted with EBV and having gastric cancer) and the remaining 51 subjects (not afflicted with EBV, but having gastric cancer) were deemed to have the second cancer condition.

Each of the 55 validation subjects were run against the trained SVM whose performance is reported in FIG. 10C and thus was assigned by the SVM to either the first or second cancer class. That is, the gene abundance values for the genes listed in FIG. 10B in which the feature type was “gene expression” and the mutation count in the two genes listed in FIG. 10B in which the feature type was “number of mutations” was measured from a tumor sample for each of the 55 validation subjects and this data for validation subject was separately input into the trained SVM model of FIG. 5C. As illustrated in FIG. 10D, the trained SVM had 75% specificity and 100% sensitivity for cancer class using such data across the 55 validation subjects. This example shows that the trained SVM model accurately predicts viral infection in tumors using RNA expression data. This example confirms viral infections are generally associated with an upregulation of immune responses. This example further shows that viral detection based on whole transcriptome data is a useful clinical tool in its own right, and further can be combined with existing diagnostic methods to provide insights about the viral status and tumor microenvironment in a single test.

### Example 10 – Obtaining Normalized RNA Count Data

In this example, patient samples were processed through RNA whole exome short-read next generation sequencing (NGS) to generate RNA sequencing data, and the RNA sequencing data were processed by a bioinformatics pipeline to generate an RNA-seq expression profile for each patient sample. Specifically, solid tumor total nucleic acid (DNA and RNA) was extracted from macro-dissected FFPE tissue sections and digested by proteinase K to eliminate proteins. RNA was purified from the total nucleic acid by TURBO DNase-I to eliminate DNA, followed by a reaction cleanup using RNA clean XP beads to remove enzymatic proteins. The isolated RNA was subjected to a quality control protocol using RiboGreen fluorescent dye to determine concentration of the RNA molecules.

Library preparation was performed using the KAPA Hyper Prep Kit in which 100 ng of RNA was heat fragmented in the presence of magnesium to an average size of 200 bp. The libraries were then reverse transcribed into cDNA and Roche SeqCap dual end adapters were ligated onto the cDNA. cDNA libraries were then purified and subjected to size selection using KAPA Hyper Beads. Libraries were then PCR amplified for 10 cycles and purified using Axygen MAG PCR clean up beads. Quality control was performed using a PicoGreen fluorescent kit to determine cDNA library concentration. cDNA libraries were then pooled into 6-plex hybridization reactions. Each pool was treated with Human COT-1 and IDT xGen Universal Blockers before being dried in a vacufuge. RNA pools were then resuspended in IDT xGen Lockdown hybridization mix, and IDT xGen Exome Research Panel v1.0 probes were added to each pool. Pools were incubated to allow probes to hybridize. Pools were then mixed with Streptavidin-coated beads to capture the hybridized molecules of cDNA. Pools were amplified and purified once more using the KAPA HiFi Library Amplification kit and Axygen MAG PCR clean up beads, respectively. A final quality control step involving PicoGreen pool quantification, and LabChip GX Touch was performed to assess pool fragment size. Pools were cluster amplified using Illumina Paired-end Cluster Kits with a PhiX-spike in on Illumina C-Bot2, and the resulting flow cell containing amplified target-captured cDNA libraries were sequenced on an Illumina HiSeq 4000 to an average unique on-target depth of 500x to generate a FASTQ file.

In this example, the cDNA library preparation was performed with an automated system, using a liquid handling robot (SciClone NGSx).

Each FASTQ file contained a list of paired-end reads generated by the Illumina sequencer, each of which was associated with a quality rating. The reads in each FASTQ file were processed by a bioinformatics pipeline. FASTQ files were analyzed using FASTQC for rapid assessment of quality control and reads. For each FASTQ file, each read in the file was aligned to a reference genome (GRch37) using kallisto alignment software. This alignment generated a SAM file, and each SAM file was converted to BAM, BAM files were sorted, and duplicates were marked for deletion.

For each gene, the raw RNA read count for a given gene was calculated by kallisto alignment software as a sum of the probability, for each read, that the read aligns to the gene. Raw counts are therefore not integers in this example. The raw read counts were saved in a tabular file for each patient, where columns represented genes and each entry represented the raw RNA read count for that gene.

Raw RNA read counts were then normalized to correct for GC content and gene length using full quantile normalization and adjusted for sequencing depth via the size factor method. Normalized RNA read counts were saved in a tabular file for each patient, where columns represented genes and each entry represented the raw RNA read count for that gene.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a non-transitory computer readable storage medium. For instance, the computer program product could contain the program modules shown in any combination in FIGS. 1 and 6 and/or as described in FIGS. 3, 5A, 5B,5C, 5D, 5E, 5F, 5G, 5H, 5L, 5J. 7A, 7B, 7C, 7D, 7E, and 8. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method of determining whether a subject is afflicted with an oncogenic pathogen, the method comprising:
(a) obtaining an amount of nucleic acid from a biological sample of the subject, wherein the amount of nucleic acid comprises nucleic acid from the subject and potentially nucleic acid from at least one oncogenic pathogen in a plurality of oncogenic pathogens;
(b) hybridizing the amount of nucleic acid to a probe set, wherein the probe set includes a plurality of nucleic acid probes for a plurality of human genomic loci and a respective set of nucleic acid probes for genomic loci of each respective oncogenic pathogen in the plurality of oncogenic pathogens;
(c) obtaining a plurality of sequence reads of the nucleic acid hybridized to the probe set in (b);
(d) determining, for each respective sequence read in the plurality of sequence reads, whether the respective sequence read aligns to a human reference genome through an alignment of the respective sequence read;
(e) determining, for each respective sequence read in the plurality of sequence reads that fail to align to the human reference genome, whether the respective sequence read aligns to a reference genome of an oncogenic pathogen in the plurality of oncogenic pathogens; and
(f) tracking, for each respective oncogenic pathogen in the plurality of oncogenic pathogens, a number of sequence reads in the plurality of sequence reads that both (i) fail to align to the human reference genome in the determining (d) and (ii) align to a reference genome of the respective oncogenic pathogen in the determining (e), thereby obtaining a sequence read count for each oncogenic pathogen in the plurality of oncogenic pathogens; and
(g) using the sequence read count for each oncogenic pathogen in the plurality of oncogenic pathogens to ascertain whether the subject is afflicted with an oncogenic pathogen.

2. The method of claim 1, wherein an oncogenic pathogen in the plurality of oncogenic pathogens is an oncogenic virus.

3. The method of claim 2, wherein:
the using (g) determines that the subject is afflicted with the oncogenic virus, and
the method further comprises using the sequence reads that map to a reference genome of the oncogenic virus to determine a strain of the oncogenic virus from among a plurality of strains of the oncogenic virus.

4. The method of any one of claims 1-3 1, wherein each oncogenic pathogen in the plurality of oncogenic pathogens is an oncogenic virus.

5. The method of any one of claims 1-4, wherein an oncogenic pathogen in the plurality of oncogenic pathogens is an oncogenic virus listed in Table 1.

6. The method of any one of claims 1-5, wherein the plurality of oncogenic pathogens includes a member of the papillomavirus family, a member of the herpes virus family, or a member of the murine polyomavirus group.

7. The method of claim 6, wherein:
the plurality of oncogenic pathogens includes the member of the papillomavirus family,
the using (g) determines that the subject is afflicted with the member of the papillomavirus family, and
the method further comprises using the sequence reads that map to a reference genome of the member of the papillomavirus family to determine a strain of the member of the papillomavirus family from among a plurality of strains of the papillomavirus family.

8. The method of claim 7, wherein the strain of the member of the papillomavirus family is HPV16, HPV18, HPV31, HPV33, HPV35, HPV39, HPV45, HPV51, HPV52, HPV56, HPV58, HPV59 or HPV68.

9. The method of any one of claims 6-8, wherein the plurality of oncogenic pathogens includes the member of the papillomavirus family, and wherein the member of the papillomavirus family is human papillomavirus (HPV).

10. The method of claim 9, wherein the HPV is HPV16 or HPV18.

11. The method of claim 9, wherein the HPV is HPV16, HPV18, HPV31, HPV33, HPV35, HPV39, HPV45, HPV51, HPV52, HPV56, HPV58, HPV59 or HPV68.

12. The method of any one of claims 6-11, wherein:
the plurality of oncogenic pathogens includes the member of the herpes virus family, and
the member of the herpes virus family is Epstein-Barr virus.

13. The method of any one of claims 6-12, wherein:
the plurality of oncogenic pathogens includes the member of the herpes virus family,
the using (g) determines that the subject is afflicted with the member of the herpes virus family, and
the method further comprises using the sequence reads that map to a reference genome of the member of the herpes virus family to determine a strain of the member of the herpes virus family from among a plurality of strains of the herpes virus family.

14. The method of claim 13, wherein the plurality of strains of the herpes virus family includes the Epstein-Barr virus.

15. The method of any one of claims 6-14, wherein:
the plurality of oncogenic pathogens includes the member of the murine polyomavirus group, and
the member of the murine polyomavirus group is Merkel cell polyomavirus.

16. The method of any one of claims 6-15, wherein:
the plurality of oncogenic pathogens includes the member of the murine polyomavirus group,
the using (g) determines that the subject is afflicted with the member of the murine polyomavirus group, and
the method further comprises using the sequence reads that map to a reference genome of the member of the murine polyomavirus group to determine a strain of the murine polyomavirus group from among a plurality of strains of the murine polyomavirus group.

17. The method of claim 16, wherein a strain in the plurality of strains of the murine polyomavirus group is Merkel cell polyomavirus.

18. The method of any one of claims 1-17, wherein an oncogenic pathogen in the plurality of oncogenic pathogens is an oncogenic bacterium.

19. The method of claim 18, wherein the oncogenic bacterium is an oncogenic bacterium listed in Table 1.

20. The method of any one of claims 1-19, wherein an oncogenic pathogen in the plurality of oncogenic pathogens is an oncogenic trematode.

21. The method of claim 20, wherein the oncogenic trematode is an oncogenic trematode listed in Table 1.

22. The method of any one of claims 1-21, wherein the plurality of human genomic loci comprises at least fifty human genomic loci.

23. The method of claim 22, wherein the plurality of human genomic loci comprises at least fifty human genomic loci selected from FIG. 4.

24. The method of any one of claims 1-21, wherein the plurality of human genomic loci comprises at least one hundred human genomic loci.

25. The method of claim 24, wherein the plurality of human genomic loci comprises at least one hundred human genomic loci selected from FIG. 4.

26. The method of any one of claims 1-21, wherein the plurality of human genomic loci comprises at least two hundred and fifty human genomic loci.

27. The method of claim 26, wherein the plurality of human genomic loci comprises at least two hundred and fifty human genomic loci selected from FIG. 4.

28. The method of any one of claims 1-21, wherein the plurality of human genomic loci comprises at least four hundred human genomic loci.

29. The method of claim 28, wherein the plurality of human genomic loci comprises at least four hundred human genomic loci selected from FIG. 4.

30. The method of any one of claims 1-21, wherein the plurality of human genomic loci comprises at least five hundred human genomic loci.

31. The method of claim 30, wherein the plurality of human genomic loci comprises at least five hundred human genomic loci selected from FIG. 4.

32. The method of any one of claims 1-31, further comprising, after the hybridizing (b) and prior to the obtaining (c), amplifying nucleic acids that bound to the probe set.

33. The method of any one of claims 1-32, wherein the plurality of sequence reads obtained in (c) have an average length of at least fifty nucleotides.

34. The method of any one of claims 1-33, wherein the alignment of the respective sequence read in the determining (d) comprises using a hash table of the human reference genome, wherein the hash table uses a seed length that is at least sixteen nucleotides in length to hash a plurality of reference seeds drawn from the human reference genome.

35. The method of claim 34, wherein the hash table uses a rolling window hash in which the plurality of reference seeds overlap each other on the human reference genome.

36. The method of claim 34 or 35, wherein the seed length is between 18 nucleotides and 22 nucleotides.

37. The method of claim 34 or 35, wherein the seed length is 20 nucleotides.

38. The method of any one of claims 34-38, wherein the determining (d) comprises:
(i) identifying one or more locations of the human reference genome that match a respective sequence read using the hash table;
(ii) determining, for each respective location of the one or more locations, a similarity score based upon a minimum edit distance between the respective location and the respective sequence read; and
(iii) making a determination as to whether the respective sequence read aligns to the human reference genome using at least the best similarity score for the one or more locations of the human reference genome.

39. The method of claim 38, wherein the one or more locations include a plurality of locations that are ranked by their minimum edit distance thereby forming a ranked list of minimum edit distances, and wherein the respective sequence read is determined to align to the human reference genome when a smallest minimum edit distance is smaller than a second most smallest minimum edit distance in the ranked list of minimum edit distances by a threshold amount.

40. The method of claim 38, wherein
the determining (d) draws a plurality of sequence read seeds from the respective sequence read and performs the identifying (i) and the determining (ii) for each sequence read seed in the plurality of sequence read seeds, and
the making (iii) requires at least three sequence read seeds in the plurality of sequence read seeds to a same candidate location of the human reference genome in order for the respective sequence read to be considered aligned to the human reference genome.

41. The method of any one of claims 1-40, wherein the determining (e) further comprises performing a procedure for each respective sequence read in the plurality of sequence reads that (i) fails to align to the human reference genome in the determining (d) and (ii) aligns to a respective reference genome of an oncogenic pathogen in the plurality of oncogenic pathogens, the procedure comprising:
calculating a corresponding similarity score between the respective sequence read and the respective reference genome of the oncogenic pathogen in the plurality of oncogenic pathogens;
labeling the respective sequence read as aligning with human reference genome when the best similarity score between the respective sequence read and the human reference genome exceeds the similarity score between the respective sequence read and the respective reference genome of the oncogenic pathogen in the plurality of oncogenic pathogens; and
labeling the respective sequence read as aligning with a particular oncogenic pathogen in the plurality of oncogenic pathogens when the similarity score between the respective sequence read and the reference genome of the particular oncogenic pathogen exceeds the best similarity score between the respective sequence read and the human reference genome.

42. The method of any one of claims 1-41, wherein the aligns to a reference genome of an oncogenic pathogen in the plurality of oncogenic pathogens in the determining (e) comprises using a corresponding oncogenic pathogen hash table of the reference genome of the respective oncogenic pathogen, wherein the corresponding hash table uses a seed length that is at least sixteen nucleotides in length to hash a plurality of reference seeds drawn from the reference genome of the respective oncogenic pathogen.

43. The method of any one of claims 1-42, wherein the using (g) identifies the subject as being afflicted with a respective oncogenic pathogen in the plurality of oncogenic pathogens when the read count for the respective oncogenic pathogen exceeds a threshold number of sequence reads in the plurality of sequence reads.

44. The method of claim 43, wherein the threshold number of sequence reads is ten sequence reads.

45. The method of claim 43, wherein the threshold number of sequence reads is between seven and twenty-five sequence reads.

46. The method of any one of claims 1-45, wherein the plurality of sequence read is obtained by next-generation sequencing.

47. The method of any one of claims 1-46, wherein the biological sample is a solid biopsy.

48. The method of claim 47, wherein the solid biopsy is a macro dissected formalin fixed paraffin embedded (FFPE) tissue section.

49. The method of any one of claims 1-46, wherein the biological sample comprises blood or saliva.

50. The method of any one of claims 1-49, wherein the subject has cancer.

51. The method of any one of claims 1-50, wherein the method is integrated with a test to determine whether the subject has a type of cancer.

52. The method of any one of claims 1-51, wherein the plurality of sequence reads are DNA sequence reads.

53. The method of any one of claims 1-51, wherein the plurality of sequence reads are RNA sequence reads.

54. The method of any one of claims 1-53, wherein the using (g) determines that the subject is afflicted with a first oncogenic pathogen in the plurality of oncogenic pathogens, and wherein the method further comprises:
subjecting the sequence reads for the first oncogenic pathogen in the plurality of sequence reads to de novo assembly thereby reconstructing a consensus sequence of a genome of the first oncogenic pathogen;
comparing the genome of the first oncogenic pathogen to the respective reference genome of each strain in one or more known strains of the first oncogenic pathogen; and
identifying the first oncogenic pathogen in the subject as a new strain of the first oncogenic pathogen when a homology between the genome of the first oncogenic pathogen and the reference genome of each strain in one or more known strains of the first oncogenic pathogen fails to satisfy a homology criterion.

55. The method of claim 54, wherein the homology criterion is ninety percent.

56. The method of any one of claims 1-55, wherein the respective set of nucleic acid probes for the genomic loci of each respective oncogenic pathogen in the plurality of oncogenic pathogens include probes collectively representing at least four of the portions of viral genomes listed in Table 2.

57. The method of any one of claims 1-55, wherein the respective set of nucleic acid probes for the genomic loci of each respective oncogenic pathogen in the plurality of oncogenic pathogens include probes collectively representing at least ten of the portions of viral genomes listed in Table 2.

58. The method of any one of claims 1-55, wherein the respective set of nucleic acid probes for the genomic loci of each respective oncogenic pathogen in the plurality of oncogenic pathogens include probes collectively representing all of the portions of viral genomes listed in Table 2.

59. The method of any one of claims 1-58, wherein the plurality of sequence reads comprises 25 million sequence reads.

60. The method of any one of claims 1-59, further comprising, after the using (g):
generating a clinical report for the subject, the clinical report indicating whether the subject is afflicted with an oncogenic pathogen in the plurality of oncogenic pathogens.

61. The method of claim 60, wherein:
the subject has cancer, and
the clinical report further indicates a type of the cancer, wherein the indicated type of the cancer is dependent upon whether the subject is afflicted with an oncogenic pathogen in the plurality of oncogenic pathogens.

62. The method of claim 61, wherein when the subject (i) has a B-cell lymphoma and (ii) is afflicted with human papillomavirus, the clinical report indicates that the type of cancer is Epstein-Barr virus-positive mucocutaneous ulcer (EBVMCU).

63. The method of any one of claims 60-62, wherein:
the subject has metastatic cancer, and
the clinical report further indicates a primary origin of the metastatic cancer, wherein the indicated primary origin of the metastatic cancer is dependent upon whether the subject is afflicted with an oncogenic pathogen in the plurality of oncogenic pathogens.

64. The method of claim 61, wherein when the subject (i) has metastatic squamous cell carcinoma (SCC) and (ii) is afflicted with human papillomavirus, the clinical report indicates that the primary origin of the metastatic cancer is the oropharynx.

65. The method of any one of claims 60-64, wherein:
the subject has cancer, and
the clinical report further indicates a recommended treatment modality for the cancer, wherein the recommended treatment modality for the cancer is dependent upon whether the subject is afflicted with an oncogenic pathogen in the plurality of oncogenic pathogens.

66. The method of claim 65, wherein:
the subject has lymphoma, and the clinical report indicates:
when the subject is determined not to be afflicted with human papillomavirus, that the recommended therapy modality is a chemotherapy or an immunotherapy; and
when the subject is determined to be afflicted with human papillomavirus, that the recommended therapy modality is anti-viral therapy.

67. The method of claim 65, wherein:
the subject has lymphoma, and
the clinical report indicates:
when the subject is determined not to be afflicted with H.pylori, that the recommended therapy modality is a chemotherapy or an immunotherapy; and
when the subject is determined to be afflicted with H.pylori, that the recommended therapy modality is antibiotics.

68. The method of any one of claims 60-67, wherein:
the subject has cancer, and
the clinical report further indicates a prognosis for the cancer, wherein the prognosis for the cancer is dependent upon whether the subject is afflicted with an oncogenic pathogen in the plurality of oncogenic pathogens.

69. The method according to any one of claims 1-68, further comprising discriminating between a first cancer condition and a second cancer condition in a human subject, wherein the first cancer condition is associated with infection by a human papillomavirus (HPV) oncogenic virus and the second cancer condition is associated with an HPV-free status by:
(A) obtaining a dataset for the subject, the dataset comprising a plurality of abundance values from the subject, wherein:
each respective abundance value in the plurality of abundance values quantifies a level of expression of a corresponding gene, in a plurality of genes, in a cancerous tissue from the subject, and
the plurality of genes comprises at least five genes selected from the genes listed in Table 21; and
(B) inputting the dataset to a classifier trained to discriminate between at least the first cancer condition and the second cancer condition based on the abundance values of the plurality of genes.

70. The method of claim 69, wherein the first cancer condition is cervical cancer associated with infection by a human papillomavirus (HPV).

71. The method of claim 69, wherein the first cancer condition is head and neck cancer associated with infection by a human papillomavirus (HPV).

72. The method according to any one of claims 69-71, wherein the plurality of genes comprises at least ten genes selected from the genes listed in Table 21.

73. The method according to any one of claims 69-71, wherein the plurality of genes comprises at least twenty genes selected from the genes listed in Table 21.

74. The method according to any one of claims 69-71, wherein the plurality of genes comprises at least all twenty-four of the genes listed in Table 21.

75. The method according to any one of claims 69-74, wherein the plurality of genes comprises at least one gene that is not listed in Table 21.

76. The method according to any one of claims 69-75, wherein the dataset further comprises a variant allele count for one or more alleles at one or more loci in the genome of the cancerous tissue from the subject.

77. The method of claim 76, wherein the one or more variant alleles are selected from variant alleles in a TP53 (ENSG00000141510) or CDKN2A (ENSG00000147889) gene.

78. The method according to any one of claims 69-77, wherein the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm.

79. The method according to any one of claims 1-68, further comprising discriminating between a first cancer condition and a second cancer condition in a human subject, wherein the first cancer condition is associated with infection by an Epstein-Barr virus (EBV) oncogenic virus and the second cancer condition is associated with an EBV-free status by:
(A) obtaining a dataset for the subject, the dataset comprising a plurality of abundance values from the subject, wherein:
each respective abundance value in the plurality of abundance values quantifies a level of expression of a corresponding gene, in a plurality of genes, in a cancerous tissue from the subject, and
the plurality of genes comprises at least five genes selected from the genes listed in Table 22; and
(B) inputting the dataset to a classifier trained to discriminate between at least the first cancer condition and the second cancer condition based on the abundance values of the plurality of genes.

80. The method of claim 79, wherein the first cancer condition is gastric cancer associated with infection by an Epstein-Barr virus (EBV).

81. The method according to any one of claims 79-80, wherein the plurality of genes comprises at all nine genes listed in Table 22.

82. The method according to any one of claims 79-81, wherein the plurality of genes comprises at least one gene that is not listed in Table 22.

83. The method according to any one of claims 79-82, wherein the dataset further comprises a variant allele count for one or more alleles at one or more loci in the genome of the cancerous tissue from the subject.

84. The method of claim 83, wherein the one or more variant alleles are selected from variant alleles in a TP53 (ENSG00000141510) or PIK3CA (ENSG00000121879) gene.

85. The method according to any one of claims 79-84, wherein the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm.

86. A method for treating cervical cancer in a human cancer patient, the method comprising:
(A) determining whether the human cancer patient is infected with a human papillomavirus (HPV) oncogenic virus by:
obtaining a dataset for the human cancer patient, the dataset comprising a plurality of abundance values, wherein:
each respective abundance value in the plurality of abundance values quantifies a level of expression of a corresponding gene, in a plurality of genes, in a cancerous tissue from the subject, and
the plurality of genes comprises at least five genes selected from the genes listed in Table 21, and
inputting the dataset to a classifier trained to discriminate between at least a first cancer condition associated with HPV infection and a second cancer condition associated with an HPV-free status based on the abundance values of the plurality of genes, in a cancerous tissue of the subject; and
(B) treating the cervical cancer by:
when the classifier result indicates that the human cancer patient is infected with an HPV oncogenic virus, administering a first therapy tailored for treatment of cervical cancer associated with an HPV infection, and
when the classifier result indicates that the human cancer patient is not infected with an HPV oncogenic virus, administering a second therapy tailored for treatment of cervical cancer not associated with an HPV infection.

87. The method of claim 86, wherein the plurality of genes comprises at least ten genes selected from the genes listed in Table 21.

88. The method of claim 86, wherein the plurality of genes comprises at least twenty genes selected from the genes listed in Table 21.

89. The method of claim 86, wherein the plurality of genes comprises at least all twenty-four of the genes listed in Table 21.

90. The method according to any one of claims 86-89, wherein the plurality of genes comprises at least one gene that is not listed in Table 21.

91. The method according to any one of claims 86-90, wherein the dataset further comprises a variant allele count for one or more alleles at one or more loci in the genome of the cancerous tissue from the subject.

92. The method of claim 91, wherein the one or more variant alleles are selected from variant alleles in a TP53 (ENSG00000141510) or CDKN2A (ENSG00000147889) gene.

93. The method according to any one of claims 86-92, wherein the classifier a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm.

94. The method according to any one of claims 86-93, wherein the first therapy tailored for treatment of cervical cancer associated with an HPV infection is a therapeutic vaccine.

95. The method according to any one of claims 86-93, wherein the first therapy tailored for treatment of cervical cancer associated with an HPV infection is an adoptive cell therapy.

96. The method according to any one of claims 86-95, wherein the second therapy tailored for treatment of cervical cancer not associated with an HPV infection is chemotherapy.

97. The method of claim 96, wherein the chemotherapy includes administration of cisplatin.

98. The method of claim 97, wherein the second therapy further comprises co-administration of a second therapeutic agent selected from the group consisting of 5-fluorouracil, paclitaxel, and bevacizumab.

99. A method for treating head and neck cancer in a human cancer patient, the method comprising:
(A) determining whether the human cancer patient is infected with a human papillomavirus (HPV) oncogenic virus by:
obtaining a dataset for the human cancer patient, the dataset comprising a plurality of abundance values, wherein:
each respective abundance value in the plurality of abundance values quantifies a level of expression of a corresponding gene, in a plurality of genes, in a cancerous tissue from the subject, and
the plurality of genes comprises at least five genes selected from the genes listed in Table 21, and
inputting the dataset to a classifier trained to discriminate between at least a first cancer condition associated with HPV infection and a second cancer condition associated with an HPV-free status based on the abundance values of the plurality of genes, in a cancerous tissue of the subject; and
(B) treating the head and neck cancer by:
when the classifier result indicates that the human cancer patient is infected with an HPV oncogenic virus, administering a first therapy tailored for treatment of head and neck cancer associated with an HPV infection, and
when the classifier result indicates that the human cancer patient is not infected with an HPV oncogenic virus, administering a second therapy tailored for treatment of head and neck cancer not associated with an HPV infection.

100. The method of claim 99, wherein the plurality of genes comprises at least ten genes selected from the genes listed in Table 21.

101. The method of claim 99, wherein the plurality of genes comprises at least twenty genes selected from the genes listed in Table 21.

102. The method of claim 99, wherein the plurality of genes comprises at least all twenty-four of the genes listed in Table 21.

103. The method according to any one of claims 99-102, wherein the plurality of genes comprises at least one gene that is not listed in Table 21.

104. The method according to any one of claims 99-103, wherein the dataset further comprises a variant allele count for one or more alleles at one or more loci in the genome of the cancerous tissue from the subject.

105. The method of claim 104, wherein the one or more variant alleles are selected from variant alleles in a TP53 (ENSG00000141510) or CDKN2A (ENSG00000147889) gene.

106. The method according to any one of claims 99-105, wherein the classifier is a logistic regression algorithm, a neural network algorithm, a convolutional neural network algorithm, a support vector machine algorithm, a Naive Bayes algorithm, a nearest neighbor algorithm, a boosted trees algorithm, a random forest algorithm, a decision tree algorithm, or a clustering algorithm.

107. The method according to any one of claims 99-106, wherein the first therapy tailored for treatment of head and neck cancer associated with an HPV infection is a therapeutic vaccine.

108. The method according to any one of claims 99-106, wherein the first therapy tailored for treatment of head and neck cancer associated with an HPV infection is an immune checkpoint inhibitor.

109. The method according to any one of claims 99-106, wherein the first therapy tailored for treatment of head and neck cancer associated with an HPV infection is a PI3K inhibitor.

110. The method according to any one of claims 99-109, wherein the second therapy tailored for treatment of head and neck cancer not associated with an HPV infection is chemotherapy.

111. The method of claim 110, wherein the chemotherapy includes administration of cisplatin.

112. The method of claim 111, wherein the second therapy further comprises concurrent radiotherapy or postoperative chemoradiation.

113. A method for treating gastric cancer in a human cancer patient, the method comprising:
(A) determining whether the human cancer patient is infected with an Epstein-Barr virus (EBV) oncogenic virus by:
obtaining a dataset for the human cancer patient, the dataset comprising a plurality of abundance values, wherein:
each respective abundance value in the plurality of abundance values quantifies a level of expression of a corresponding gene, in a plurality of genes, in a cancerous tissue from the subject, and
the plurality of genes comprises at least five genes selected from the genes listed in Table 22, and
inputting the dataset to a classifier trained to discriminate between at least a first cancer condition associated with EBV infection and a second cancer condition associated with an EBV-free status based on the abundance values of the plurality of genes, in a cancerous tissue of the subject; and
(B) treating the gastric cancer by:
when the classifier result indicates that the human cancer patient is infected with an EBV oncogenic virus, administering a first therapy tailored for treatment of gastric cancer associated with an EBV infection, and
when the classifier result indicates that the human cancer patient is not infected with an EBV oncogenic virus, administering a second therapy tailored for treatment of gastric cancer not associated with an EBV infection.

114. The method of claim 113, wherein the plurality of genes comprises at least all nine of the genes listed in Table 22.

115. The method according to any one of claims 113-114, wherein the plurality of genes comprises at least one gene that is not listed in Table 22.

116. The method according to any one of claims 113-115, wherein the dataset further comprises a variant allele count for one or more alleles at one or more loci in the genome of the cancerous tissue from the subject.

117. The method of claim 116, wherein the one or more variant alleles are selected from variant alleles in a TP53 (ENSG00000141510) or PIK3CA (ENSG00000121879) gene.

118. The method according to any one of claims 113-117, wherein the classifier is a multivariate logistic regression algorithm, a neural network algorithm, or a convolutional neural network algorithm.

119. The method according to any one of claims 113-117, wherein the classifier is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

120. The method according to any one of claims 113-119, wherein the first therapy tailored for treatment of gastric cancer associated with an EBV infection is an immune checkpoint inhibitor.

121. The method according to any one of claims 113-120, wherein the second therapy tailored for treatment of gastric cancer not associated with an EBV infection is chemotherapy.

122. The method according to claim 121, wherein the chemotherapy includes administration of a therapeutic agent selected from the group consisting of paclitaxel, carboplatin, cisplatin, 5-fluorouracil, and oxaliplatin.

123. The method according to claim 121, wherein the chemotherapy includes administration of paclitaxel and carboplatin.

124. The method according to claim 121, wherein the chemotherapy includes administration of cisplatin and 5-fluorouracil.

125. The method according to claim 121, wherein the chemotherapy includes administration of oxaliplatin and 5-fluorouracil.

126. The method according to any one of claims 69 to 125, further comprising:
determining the plurality of abundance values by RNA sequencing of a sample of the cancerous tissue from the human cancer patient.

127. The method according to any one of claims 69 to 125, wherein the cancerous tissue from the subject is a tumor sample from the subject.

128. A non-transitory computer-readable storage medium having stored thereon program code instructions that, when executed by a processor, cause the processor to perform a method for determining whether a subject is afflicted with an oncogenic pathogen, the method comprising:
(a) obtaining a plurality of sequence reads, in electronic form, of an amount of nucleic acids from a biological sample of the subject, wherein the amount of nucleic acid comprises nucleic acid from the subject and potentially nucleic acid from at least one oncogenic pathogen in a plurality of oncogenic pathogens;
(b) determining, for each respective sequence read in the plurality of sequence reads, whether the respective sequence read aligns to a human reference genome through an alignment of the respective sequence read using a non-local alignment method;
(c) determining, for each respective sequence read in the plurality of sequence reads that fail to align to the human reference genome using the non-local alignment method, whether the respective sequence read aligns to a reference genome of an oncogenic pathogen in the plurality of oncogenic pathogens; and
(d) tracking, for each respective oncogenic pathogen in the plurality of oncogenic pathogens, a number of sequence reads in the plurality of sequence reads that both (i) fail to align to the human reference genome in the determining (b) and (ii) align to a reference genome of the respective oncogenic pathogen in the determining (c), thereby obtaining a sequence read count for each oncogenic pathogen in the plurality of oncogenic pathogens; and
(e) using the sequence read count for each oncogenic pathogen in the plurality of oncogenic pathogens to ascertain whether the subject is afflicted with an oncogenic pathogen.

129. A non-transitory computer-readable storage medium having stored thereon program code instructions that, when executed by a processor, cause the processor to perform a method according to any one of claims 1-85.

130. A computer system for determining whether a subject is afflicted with an oncogenic pathogen, the computer system comprising:
at least one processor, and
a memory storing at least one program for execution by the at least one processor, the at least one program comprising instructions for:
(a) obtaining a plurality of sequence reads, in electronic form, of an amount of nucleic acids from a biological sample of the subject, wherein the amount of nucleic acid comprises nucleic acid from the subject and potentially nucleic acid from at least one oncogenic pathogen in a plurality of oncogenic pathogens;
(b) determining, for each respective sequence read in the plurality of sequence reads, whether the respective sequence read aligns to a human reference genome through an alignment of the respective sequence read using a non-local alignment method;
(c) determining, for each respective sequence read in the plurality of sequence reads that fail to align to the human reference genome using the non-local alignment method, whether the respective sequence read aligns to a reference genome of an oncogenic pathogen in the plurality of oncogenic pathogens; and
(d) tracking, for each respective oncogenic pathogen in the plurality of oncogenic pathogens, a number of sequence reads in the plurality of sequence reads that both (i) fail to align to the human reference genome in the determining (b) and (ii) align to a reference genome of the respective oncogenic pathogen in the determining (c), thereby obtaining a sequence read count for each oncogenic pathogen in the plurality of oncogenic pathogens; and
(e) using the sequence read count for each oncogenic pathogen in the plurality of oncogenic pathogens to ascertain whether the subject is afflicted with an oncogenic pathogen.

131. A computer system for determining whether a subject is afflicted with an oncogenic pathogen, the computer system comprising:
at least one processor, and
a memory storing at least one program for execution by the at least one processor, the at least one program comprising instructions for performing a method according to any one of claims 1-85.
